SEQUENCE LISTING (262 sequences)

SEQ ID NO: 1 - DNA, 801 bp, Bovine
agtgcacgtgtctggacgggtcggtgggctgcgtgcccctatgcagcgtggacgtccgcctgcccagccccgactgccccttccctcggagggtcaaactgcccgggaaatgctgcgaggaatgggtgtgtgatgagcccaaggagcacaccgtggtcggccctgcgctcgcagcttaccggccggaagacacgtttggcccagacccaaccatgatccgagccaactgcctggtccagaccacagagtggagtgcctgttccaagacctgcggaatgggcatctccacccgggttaccaatgacaacgcattctgcaggctggagaagcagagccgcctctgcatggtcaggccttgcgaagctgacctggaggagaacattaagaaaggcaaaaagtgcatccggacccccaaaatctccaagcctatcaagtttgagctttctggctgcaccagcatgaagacataccgagctaaattctgcggagtgtgcacagacgggcggtgctgcaccccccacagaaccaccacccttcccgtggagttcaagtgtcctgatggggaggtcatgaagaagagcatgatgttcatcaagacctgtgcctgccattacaactgccccggagacaatgacatcttcgagtcactgtactacaggaagatgtatggagacatggcctaaagccagagacagtgagacacgtgaacattttaggctgtcacttgaatcgattcacatctcatttttgtgtacacgtgatttcagtggcacaagttatttaaatctgtgcttctaactg

SEQ ID NO: 2 - DNA, 511 bp, Bovine
gtggagtcggttgggcccttctcttgtggacttttccagttcggcctttcgaaacgtctgcacgcggaaaccaaccgccaggaaagaggcgcggcgtcgagccgagctaacctgaggaaaccgctgccgctgcagccgagcgcgtgggcccgccggggcattagttcgtctgcgcgcagccccggcccgactttccagagccatgagttacggtcgcccgcctcccgatgtggagggcatgacctctctcaaggtggataacctaacctaccgcacctcacccgacaccctgaggcgcgtgttcgagaagtacgggcgcgtcggcgatgtgtacatcccgcgggaccgctacaccaaggagtcccgcggcttcgccttcgtccgcttccacgacaagcgcgacgccgaggacgccatggatgccatggacggagccgtactggacggccgcgagctgcgggtgcagatggcgcgctacggccgcccccggattcgcaccatagccgccggg

SEQ ID NO: 3 - DNA, 801 bp, Bovine; identical to SEQ ID NO: 1

SEQ ID NO: 4 - DNA, 657 bp, Bovine
tttccaccgtacgccggtgctgggagtgcctgccttctcttgtcttgaaaacctcctctttggacccagcaccgccgtcctcacggtgatgttggactcagtgacacacagcaccttcctgcccaacacgtccttctgcgaccccctgatgtcgtggactgacctgttcagcaatgaagagtattaccctgcctttgagcatcagacagcttgcgactcctactggacatccgtccaccccgaatactggacgaagcgccacgtctgggaatggctccagttctgctgtgaccagtacaagctggacgccaactgcatctctttctgccatttcaacatcagtggcctgcagctgtgcggcatgacacaggaggagttcatggaggcggccggcgtctgtggggagtatctgtactttatcctccagagcatccgctcacaaggttactccttttttaatgatcctgatgagaccaaggccaccctcaaagactatgctgattccagttgcttgaaaacaagtggcatcaaaagtcaagactgtcacagtcatagtcgaacaagcctccagagttctcatctatgggaatttgtgcgagatctgcttctatctcctgaggaaaactgcggcattctggaatgggaagataaggaaca

SEQ ID NO: 5 - DNA, 1107 bp, Bovine
gcaaagtgactcagttatacctatgcatgtatctcttctttttcaaattcttttcccatttggctgttacataattatgaaactatacccctgtctcagactgctgcagcctgagttctaatctaaccctgcattgatttcatgctttgctttctgctcttcataagctgtagtccctttgcttttccctgtgtcctttctggtctgttctccacactagccggagggatcttcttaacgtgtggatcttgtcaaagtcttccgtcttcacttgttgaataaaatccctcgccattctcaggtgcccacgtgacttgtcagcagctagctgcccccgaggcaacacctcccaccctccctagcacccggaggtcccctgggctttgcactgctgctccttccctctgaaaccctctcccacctcctgacgttagcatgaatgtgttcctctaggagacccgctctgcccaccccatctagatgcagcaggagctttcgcctgcctccattctctaatgcctccttccttcgtaacacttacaacaggggcgattcgtgtttcacttgtattttcctcaccctgtagtcctcagcctctacctgagcgaggaccaagtctgccttgtctccagctttctgtttccagctcagaacctggaatctgacacacagttgggccccagtgttgaatacaaattctcctgaaacctactaattatccccagaacagccggcaccagttgtctcctgccacgaaggtgtccaattaatagctgctcatttggtgtgacttgaggtctgaatgtggcctctagatttcttcctgcagctgtcaatcactctgttgcagatgctgattccagttgcttgaaaacaagtggcatcaaaagtcaagactgtcacagtcatagtcgaacaagcctccagagttctcatctatgggaatttgtgcgagatctgcttctatctcctgaggaaaactgcggcattctggaatgggaagatagggaacaaggtatttttcgggtggttaaatcagaagccctggcgaagatgtggggacaaaggaagaaaaatgacagaatgacgtacgaaaagctgagcaga

SEQ ID NO: 6 - DNA, 305 bp, Bovine
ctggcaactctggctcagagagtgaaggaggttttaccccatgttccactgggcgtcatccagagagacctagccaggactggctgtgtagacttgaccatcactaatctgcttgagggagccgtagctttcatgcctgaagacatcactgaggggacccaatcccttgccacagcctccactcccaagttccccagctctggcccggcaacccctcagcccacagccctaacatttgccaagtcctcctgggcccggcaggagagtctacaggagcgaaagcaggcgctgtatgaatgcgcgag

SEQ ID NO: 7 - DNA, 220 bp, Bovine
ctccttccctcagaggatgtcctccttccagctcaacctcaacccgctcaaggagcctctcggcttcatcaaagtcctcgagtggattgcttctatctttgcttttgctacctgtggaggttttaagggcaaaacagaaattcaagtgtcttgtactacgggtcctgagaataaaacgattacagctgcttttggttatccattcaggttgaatgaagca

SEQ ID NO: 8 - DNA, 565 bp, Bovine
accaggcaacccagaaagccaggcgtggagactgatcctgcgggaggaaagggttcatcatggcggatgatctaaaacgattcctgtataaaaaattaccgagtgttgaggggctccatgctattgttgtgtcagatagagatggagtgcctgtcatcaaagtggccaatgataatgctccagagcatgctttgagacctggtttcttatcaacttttgcccttgcaacagaccaaggaagcaaactcggactttcaaaaaataaaagtatcatctgttactataatacctaccaggtggttcaattcaatcgtttacctttggtagtgagtttcatagccagcagcaatgctaatacaggactaattgtcagcctggaaaaggaacttgctccattatttgaagaattgagacaagttgtggaagtttcttaatctggagttttcttcatcatatcagacacaatatcaatccagcaatctttaggccacagtgacacttgtatccatgtactcaaggaccccctttttccactttactctagaaaaagagccttacagataga

SEQ ID NO: 9 - DNA, 436 bp, Bovine
cagccggccaagctggccgaggccttcaagtacttcgtgcagggcatgggatacatgccctccgccagcatgacccgcttgatgaggtctcgcacagcgtcaggctccagcgtcacgtccctggagggtgcccgcagccgctcgcataccagcgagggcactcgcagccgctcgcacaccagcgagggcacgcggctggacatcatccccaactccggcggccccgggagcagcgccgggcccaactccacggaggtgtcctgctaggcggcctcggggccggccggcctcccctggactctgggaccgatcgctgtagctgcccctcctccggcccccctccccacccctgcctgccaaccgcacgttaactcgtaactcggtattaatccaaagcttatttgtacaagtgagctctggtgacgacacggtgaga

SEQ ID NO: 10 - DNA, 315 bp, Bovine
aaggaatccaagaggggtcaggcgtcgccgtcgccctgaagacggaggaggggcaagatggtttacatctcgaatggacaagtgttggacagcaggagtcagtccccatggagattatcttttataacagatttcttctggggaatagctgagtttgtggttttgtttttcagaactctgcttcaacaagatgtgaaaaagagaagaggctacggaagctcatctgattccagatatgatgacggaagagggccaccaggaaacccccccagaagaagaatgggtcgaattaatcatctgcagggccctaatcct

SEQ ID NO: 11 - DNA, 333 bp, Bovine
gaaagacccagcactgggaggctaaggaaaagaggtgagtcaaggacgtcttcgtagtggagctactgagattcgggaggccacgctgggcttgtttggcgtccaaacgacttggacagttcagtcatgttcggctacgcggtgaggcgcgccctgcgcaagagtaagacccttcgctacggagttcccatgttgttgctgattgttggaggttcttttggtcttcgtgagttttctcaaattcgttatgatgctgtgaagattaaaattgatcctgagttagaaaaaaagctgaaaatgaataaagtgtcattggaatcagaatatgagaaa

SEQ ID NO: 12 - DNA, 930 bp, Bovine
ggatgacccagatcatgttcgagaccttcaacacccctgccatgtacgtggccatccaggctgtgctgtccctgtatgcctctggccgcaccaccggcatcgtgatggactccggtgacggggtcacccacacggtgcccatctatgaggggtacgcccttccccatgccatcctgcgtctggacctggctggccgggacctgacggactacctcatgaagatcctcacggagcgtggctacagcttcaccaccacggccgagcgggaaatcgtccgtgacatcaaggagaagccttgctacgtggccctggacttcgagcaggagatggccaccgcggcctccagctcctccctggagaagagctacgagcttcctgacgggcaggtcatcaccatcggcaatgagcggttccgctgccctgaggctctcttccagccttccttcctgggcatggaatcctgcggcattcacgaaactaccttcaattccatcatgaagtgtgacgtcgacatccgcaaggacctctacgccaacacggtgctgtccggcgggaccaccatgtaccccggcatcgcggacaggatgcagaaagagatcactgccctggcacccagcacaatgaagatcaagatcatcgcgccccctgagcgcaagtactccgtgtggattggcggctccatcctggcctcgctgtccaccttccagcagatgtggatcagcaagcaggagtacgatgagtccggcccctccatcgtccaccgcaaatgcttctaggcggactgttagctgcgttacaccctttttcttgacaaaacctaacttgcgcagaaaacgagatgagattggcatggctttatttgttttttttttttgtcttttttgattttttttttttggcgcttgactcaggatttaaaaactggaacgg

SEQ ID NO: 13 - DNA, 639 bp, Bovine
gaagaccgtggccgtgccctgcataattcaagactcaagttcatgttgcgtgccgaactgcgagcccagcctgtccgtgcagccaccagccctcgaggacctgctcctgggctccaacgccagcctcacgtgcacactgagtggcctgaaaagcgccgagggcgccagcttcacctggaacccgacaggtgggaagaccgccgtccaggggtcgcccaagcgtgactcctgtggctgctacagcgtgtccagcgtcctgccgggctgtgccgatccctggaacagtggacagactttctcctgctctgtcacccaccccgagtccaagagttcactgaccgccaccatcaagaaagacttagggaacacgttccggcctcaggtccacctgctgccgccgccgtcggaggagctggccctcaacgagctggtgacgctgacgtgcctggtgcggggattcaaccccaaggaggtgctggtgcgttggctgcagggcaatcaagagctgccccgcgagaagtatctgacctgggccccctgcccgagctggccagagcgtaccaccttcgccgtgaccaacgtgctacgcgtggacgccgaggtctggaagcagggggacaccttctctgcatggtgggc

SEQ ID NO: 14 - DNA, 565 bp, Bovine
cccgactcgacgtcgtcgttacagggaagaagaagcgggtgagaaaaacttctgtttccaccgttttgcccatttctgcagatttgttccgaggccgaggagcctttgttggaagagatggtcatggtcctgagccccctgtttttggtcttcatactgggtctgggtctgaccccagtggccccggctcaagatgactacagatacatacacttcctgacccagcactacgatgccaaaccaaagggccggaatgacgaatattgttttaacatgatgaaaaatcgacgcctgaccagaccttgcaaagaccgcaacacctttattcatggcaacaagaatgacattaaggccatctgtgaggacagaaatggacagccttacagaggcgatctcagaataagcaagtctgaattccagatcaccatctgcaagcataaaggaggttcctcccggcctccatgccggtacggagccacagaagactccagagtcattgttgtcggctgtgaaaatggcttgcccgtccactttgatgagtcctttatcactccacgccactagc

SEQ ID NO: 15 - DNA, 421 bp, Bovine
gcgattcatgctgctgttcagccggcaggggaagctgcggctgcaaaaatggtacctggccacctcagacaaggagcggaagaagatggttcgggagcttatgcaggtggttctggctcgcaagcccaagatgtgcagcttcctggagtggagggacctcaaagttgtctataagagatacgccagcctctacttctgctgtgccatcgagggccaagacaatgagctcatcacgctggagctgatccaccgatacgtggagctcctggacaaatactttggcagcgtttgcgagctggacatcatcttcaattttgagaaggcctacttcatcttggatgagtttctgatggggggagatgtccaggacacctccaagaagagtgtgctgaaggccatcgagcaggcggacctactgcag

SEQ ID NO: 16 - DNA, 504 bp, Bovine
gggtgcaggtaatctgcatgaaggggaaggccaagtataaggccagtgagaacgccatcgtgtggaagatcaagcgcatggcaggcatgaaggaatcacaaatcagcgctgagattgagctgctgcccaccaacgacaagaagaaatgggctcgaccccccatttcaatgaactttgaggtgccattcgcaccttctggtctcaaggtgcgctacttgaaggtgtttgaaccgaagctgaactacagcgaccacgatgtcatcaaatgggtgcgctacattggccgcagcggcatttatgagacccgctgctagctgcctggtggccgctagcccacctccccacccaccctcttccacaggtctgggtgcccttggccaccacacatcagtgtctcctccctcctgctttgctgcctgccctttgcactagcccccgagtctaggtctggaccaaccacattgcaagtgggactggtggagcagtccctgggctccctgaatg

SEQ ID NO: 17 - DNA, 644 bp, Bovine
ccagccgccgccatgatcctgctggaggtgaacaaccgcatcatcgaggagacgctcgcgctcaagttcgagaacgcggccgccggaaacaaaccagaagctgtagaagtaacatttgcagattttgatggagtcctcttcagccacagggaacctcccttagagctgaaagataccgatgccgccgtgggtgacaacattggctacattaccttcgtgctgttccctcgccacaccaacgccagtgctcgagacaacaccatcaacctgatccacacgttccgggactacctgcactaccacatcaagtgctcgaaggcctatattcacacacgtatgcgggcaaaaacatccgacttcctcaaggtgctgaaccgtgcacgcccagatgccgagaaaaaggaaatgaaaacaatcacggggaagacgttttcatcccgctaactcttgggaacaggaagaggaagcggctggcaactgaaggctggaacacttgctactggataatcgtaagcttttaatgttgcacctcttcaggttcttaagggattctccgttttggttccattttgtacacgtttggaaaataatctggcaaaaacgagctgtgcttgcaaggacttcatggttccaagaattaaaag

SEQ ID NO: 18 - DNA, 375 bp, Bovine
ctttatgacacatcctgagtttaggatagaagattcagagcctcatattccccttattgatgacactgatgctgaagatgatgctcctacaaaacgtaactccagtcctccaccctctcccaacaaaaataacaatgctgttgacagcgggatttaccttacaatagaaatgaacaagtctgctacctcttcatccccaggaagcccactacatagtttggaaacatcactctgattgtaagctgaacgttaacacactagctgcattgtaaagaaacaaattgaaactgggtcttttcacatattgtgacggacaagatagtattcttgtctctggacttcaacagaagacatcttgaccaatgtagatttatt

SEQ ID NO: 19 - DNA, 596 bp, Bovine
gagacacgtcaacatgactgacaatgatcttatcaatattcttgaccctttttatcatctttcaactaaaagtttcaaaacacaacttttatcacaatccagaactgacaccaacaaaaatattaaaacaaaacaccccttgagaaacaaaatgaacgaaaatttatttacctcttttattacccctgtaattttaggtctccctctcgtaacccttatcgtactattcccaagcctactattcccaacatcaaaccgactagtaagcaatcgctttgtaaccctccaacaatgaatacttcaacttgtatcaaaacaaataatgagtatccacaattctaaaggacaaacatgaacattaatattaatatctctgatcctatttattggatcaacaaacctactaggcctattaccccattcattcacaccaacaacacaactatcaataaacctaggcatagccatccccctgtgagcaggagccgtaattacaggattccgcaataaaactaaagcatcacttgcccatttcttaccacaaggaacacccactccactaatcccaatactagtaattattgaaactatcagcc

SEQ ID NO: 20 - DNA, 296 bp, Bovine
cgagggtcacggacagtatggttccgccggtgcaggtctctccgctcatcaagctcggccgttactccgccctgttcctcggcatggcctacggcgccaagcgctacaattacctgaaacctcgggcagaagaggagaggaggcttgcagccgaggagaagaagaagcgggatgagcagaagcgcatcgagcgggagctggcggaagcccaagaggataccatattgaagtgagcctgcccttctcctgagcagtgtggctgaataaagttttctgtgctcaaaaaaaaaaaaaaa

SEQ ID NO: 21 - DNA, 669 bp, Bovine
ccgaggcgcagcagaggagggtccaggagacggaggtgaagccatgtggcagctgctgctccccctggccctggggctgggcaccatgggcttgggcagggcggagctcacgacggcccagcaccggggcctgcaggtggccctggaggagttccacaagcatccacccgtgctgtgggccttccaggtgaccagcgtggacaatgcggcagacacgctcttcccggctgggcagtttgtgaggctggagttcaagctccagcagacgagctgtcggaagaaagactggaggaaagaagactgcaaagtcaagcccaacgggagaaagcggaaatgcctggcctgcatcaagctggactcaaaagatcaagtcctgggccggatggtgcactgtcccatacagactcaggagctggacgacgcccaggacgcccagtgcagcagggtggagcgcgccggcgaggacccccacagctactacctccccggacagtttgccttcatcaaagccttgtccccctgagctgaggcctggcagaagtcacccggcttcctggaaggaagggaggtcgccagtgaaagcccgcctccctcctctgggcccggggaggggccaccccctgacccctgagctaataaagctgtgctcagctgaaaaaaaaaaaaaaa

SEQ ID NO: 22 - DNA, 558 bp, Bovine
ctgccgtccatcacatcctgtttgttctaccaccgtgtcctgtgtgtctgctgaggggagtgcccagcggggtccagggccttggcctccgtgcccagctgcgtgttgcggtgagtggtggagagctacagctctggctctcctgtcatcactggatgctctgcaggtctgtgtctgcacctgtggaagggcctgggcctggccctgtttccttgctgggaagcacgtgggtccgggggtggctgggcccctgagatgcacctcaggggcagggggggaccccagcccaccccgtgagactgaactttcctcaaacatgatggtcctcaatgacattttaacttcttttgatgaaaactgtcactttagcatgtagagtaacctattacagaatcctgtgcagtgattctagaatctctaaattgtatgatgtgttatataagaatttatttgctatcgacattcccgtgtaaaggagagacatatcatgctgctgtaatgaatttgtgtcaagatgatccaataaacttgcgaaacaggcaaaaaaaaaaaaaaaaa

SEQ ID NO: 23 - DNA, 300 bp, Bovine
gaagaatggtcctgctgcatccggaacctccttcttggccaggagaaagatgtagaggtttccataccagcctccttcttcccaaggctgactccctggatggtggctgtggctgtcatcttggtggtcctaggacttctcacaattgggtccatattttttacctggagactatacaaggaaagatccagacagaggaggaatgaattcagctctaaagagaaactcctggaagagctcaaatggaaaagggctacattgcatgcagtggatgtgactctggatccagatactgcccac

SEQ ID NO: 24 - DNA, 331 bp, Bovine
cggcagacccgcactacacttggttgctctgaacacgccgttttctggggacattcgagctgatttccagtgcttccagcaggccagggctgcaggactgttgtccacctaccgagcattcttatcctcccatttgcaagatctctccacagttgtaaggaaagcagagagatatagccttccaatagtgaacctcaagggccaagtactttttaataattgggactcaattttttctggccacggaggtcagttcaatacacacattccaatatattcctttgatggcccagatgtaatgacagatctttctggcccagaaggcattttg

SEQ ID NO: 25 - DNA, 747 bp, Bovine
ggcggcatttctctcttttccggttatggcggcgtagggaactatgagcagcaaagtctcccgcgacaccctctacgaggcggtgcgggaagtcctgcacgggaatcagcgcaagcgcagaaagtttttggagacggtggagcttcagatcagcctgaagaactatgaccctcagaaggacaaacgcttctcgggcaccgtcaggcttaagtccactccccgccccaagttctccgtgtggtcttgggggaccagcagcattgtgatgaggccaaggctgtggatatcccccacatggacatcgaggcgctgaaaaaactcaacaagaataagaaactggtcaagaagctggccaagaaatatgatgcctttttggcttcagagtctctgatcaagcagatcccccgaatcctgggcccgggcctgaacaaggctggcaagttcccttccttgctgacccacaatgagaacatggtggccaaagttgatgaagtgaagtccacgatcaagttccagatgaagaaggtgctgtgtctggcagtggctgttggccacgtgaagatgacagatgatgagcttgtgtacaacatccacttagctgtcaacttcctggtgtcattgctcaagaaaaattggcagaacgtcagggccttgtacattaagaacaccatgggcaagccccaacgtctgtactaaggcacagcttaataaacctactaaaccatcaaaaaaaaaaaaaaaaaaaaa

SEQ ID NO: 26 - DNA, 589 bp, Bovine
ggcggtactggcccagtcgtcactggcgcagaattgctgacggaacgggccgaggctggctggctgtgggaagagaggcgaggttcaggtcttctacggttgcagtgaaagtttcttgaaaatcttgttttaattgagtctttaataaatacaacgtaaaaatggcttcaaaaagagctctagtcatcctggctaaaggagcagaggaaatggagacggttatccctgtagatgtcatgagacgagctggaattaaggtcaccgttgcaggtctggctggaaaagacccggtacagtgtagccgagatgttgtcatttgtcctgatgccagtctggaagatgcaaaaaaagagggaccttatgatgtggtggttcttccaggaggtaatctgggtgcacagaatttatccgagtccgctgctgtgaaggagatactgaaggaacaagagaagaggaagggcctcatcgctgccatctgtgcaggtcctacagctctgctggctcatgaaataggttttggaagcaaagttacaacacacccacttgctaaagacaaaatgatgaacggaagtcattacagctactccga

SEQ ID NO: 27 - DNA, 333 bp, Bovine
caagcctcagttcatcagcagaggaaccttcaaccctgaaaagggcaaacaaaaattaaagaatgtgaaaaactcacctcagaaaaccaaagagaccccagaggggatagttgtgtctagccgcaggaaaactgtggacccagactgcagctcggcccaacagctagctctctttgggaataatgagtttatggtctgaactggcagatgtgtgtcccttatggctacatctcatcttggggcttcatcacctggtccacagtatccatcctgattgtggtcctgcctcttgcccgagcatacaaccaagaccttgtgtgctgagtcccgggt

SEQ ID NO: 28 - DNA, 375 bp, Bovine
gcgagctcagcgacacaagtacataaataaaggataaagtatttacagaacaaatcttcaatcaagtataacattttgatgcttggcagctaaactccttgtgccctcactatgccagcagcaactgtagatcatagccaaagaatttgtgaagtttgggcttgcaacctggatgaagagatgaagaaaattcgtcaagttatccgaaaatataattacgttgctatggacaccgagtttccaggcgtggttgcaagacccattggagaattcaggagcaatgctgactatcagtaccaactgttgcggtgtaatgtagacttgttgaagataattcaactaggactgacatttatgaatgagcaggagaatacc

SEQ ID NO: 29 - DNA, 575 bp, Bovine
gccgtcagcattcttagtctgggaggacctgcttgttctatcacaatgaactggctggtgtgggcactcctgctgtgctcctctgcaatggcacatgtgcacagagaccccactctggatcatcactgggatctctggaagaaaacctatggaaaacaatacaaagaaaagaatgaggaagtagcacggcgtctcatctgggaaaagaatctaaaaactgttacacttcacaatctggagcattcaatgggaatgcattcatatgagctaggcatgaaccacctaggagacatgaccagtgaagaagtgatatctttaatgagttccttgagagttcccagccaatggccaagaaatgtcacttacaagtcagaccctaatcagaaattgcctgattctatggactggagagagaaggggtgtgttactgaagtgaaataccagggtgcttgtggttcttgctgggctttcagtgctgtgggagccctggaagcacaagtgaagctgaaaacaggaaagctggtgtctctgagtgcacagaacctggtggattgctcaactgcaaaatatgggaa

SEQ ID NO: 30 - DNA, 315 bp, Bovine
agggacatctctacactgttcccatccgggaacagggcaacatctacaagcccaacaacaaggctatggcagaggaaatgaacgagaagcaagtgtacgacgcgcacaccaaggagatagatctggtcaaccgcgaccccaagcatctcaacgacgacgtggtcaagattgattttgaagatgtgattgcagaaccagaaggaacacacagtttcgatggcatctggaaggccagcttcaccaccttcactgtgacaaagtactggttttaccgtttgctgtctgcctctttggcatcccaatggcactcatctg

SEQ ID NO: 31 - DNA, 1220 bp, Bovine
tatacctaacgtcgttctctccacttgtcttcttcccacagataaagcagataacttcatcctgccccaaagaccaacacgatggcattattcacggtggttttgttcctggctgctgtgtggcttccattctttcctgcaaagggacaggatcgacgttttgctgatttgtcaaacaccctgaaaaatgtccaaactgagattgtaaacaaacacaatgacctaaggagaggagtctcgccacctcccagtaacatgctgaagatgcaatggaacaccacggcagcagcaaatgcccaaaattgggcaaacaagtgccttttcaaacacagtaagaaagaggatagaagagtaggtacaaggaactgtggcgagaatctctttatgtcaagttacccttctacatggtctaatgcaatccaaagctggtatgatgaggtccatgattttgtttttgaagtagggccgaagagtcctcaagcagtaattggacatttcacccagattgtttggtactcatctttccttattggatgtggagttgcctactgccccaaacaaagtctaaagtacctctatgtttgccaatactgtcctgctggtaatattgttggcagacaacatgtcccttaccaaaagggaacaccttgtggcagctgccccaatcattgtgacaacggactatgcaccaatagttgtgagtatgaagatacctattctaactgtgcatctttaaaggaaacatggacctgtgcctctgattttgtgaagaccaattgcaaggctgcctgcaattgtcaaggcaaaatttattaaattccaagcactgaccgagcagggctacatgatggaaggctgcatcatctactcagatttgatatttactaacaaggaaatcacagacatgttagctacaaatttgatttcaagtagtaaagagtctttttctcctggatctgctttttattttacagaattttttttcatacaaaaaaaattaatgtaacctaatctatgataacaactttggattttgatatcaattgggtgatgtaaatttaattgaatttaatcaagttgaagattctgaaagttgtattctcttacaaatatgatcactacaaatttgaactgaaattgagaatcatgtataaaatcaacaagctacaagtatattttgcatggcacagggcatgtagccaatattctataataactat

SEQ ID NO: 32 - DNA, 775 bp, Bovine
agtgcacgtgtctggacgggtcggtgggctgcgtgcccctatgcagcgtggacgtccgcctgcccagccccgactgccccttccctcggagggtcaaactgcccgggaaatgctgcgaggaatgggtgtgtgatgagcccaaggagcacaccgtggtcggccctgcgctcgcagcttaccggccggaagacacgtttggcccagacccaaccatgatccgagccaactgcctggtccagaccacagagtggagtgcctgttccaagacctgcggaatgggcatctccacccgggttaccaatgacaacgcattctgcaggctggagaagcagagccgcctctgcatggtcaggccttgcgaagctgacctggaggagaacattaagaaaggcaaaaagtgcatccggacccccaaaatctccaagcctatcaagtttgagctttctggctgcaccagcatgaagacataccgagctaaattctgcggagtgtgcacagacgggcggtgctgcaccccccacagaaccaccacccttcccgtggagttcaagtgtcctgatggggaggtcatgaagaagagcatgatgttcatcaagacctgtgcctgccattacaactgccccggagacaatgacatcttcgagtcactgtactacaggaagatgtatggagacatggcctaaagccagagacagtgagacacgtgaacattttaggctgtcacttgaatcgattcacatctcatttttgtgtacacgtgatttcagtggcacaa

SEQ ID NO: 33 - DNA, 652 bp, Bovine
ggacgccgccacctcggagcttcccttgccgtttagccatggtcaaccccaccgtgttcttcgacatcgctgtcgacggcgagcccttgggccgcgtctcttttgagctgtttgcagacaaagttccaaagacagcagaaaactttcgtgctctgagcactggagagaaaggatttggttataaaggttcctgctttcacagaataattccgggatttatgtgccagggtggtgacttcacacgccataatggtactggtggcaagtccatctatggcgagaaatttgatgatgagaatttcattttgaagcatacaggtcctggcatcttgtccatggcaaatgctggccccaacacaaatggttcccagtttttcatttgcactgccaagactgagtggttggatggcaagcacgtggtctttggcaaggtgaaagagggcatgaatattgtggaagccatggagcgctttgggtccaggaatggcaagaccagcaagaagatcaccattgctgactgtggacaaatctaataaatttgacttgtggtttacttaagcaccagaccattccttcctgtagcccaagaaaacaccccttcaccccatgtgcttgaaatatcctataatctttgtgctcttgctacagtctt

SEQ ID NO: 34 - DNA, 382 bp, Bovine
cacgagttagaacgaacagggcattacttaactgtgaaagataaccaagtggttcagttgcatccctctactgttcttgaccacaagcctgaatgggtgctttataatgagtttgttcttacaacaaagaattacatccggacgtgtacagacatcaagcccgaatggttggtgaaaattgcccctcaatattatgacatgagcaatttcccacagtgtgaagcaaagagacagttggaccgcatcattgccaaactgcaatccaaggaatattcacagtactgaaattcaatgcttagaactgaaattattcagaggacagctttaaaagatgaacgaactgaaaagttcaagttgtgctcttcatgttgggtcaataatg

SEQ ID NO: 35 - DNA, 315 bp, Bovine
cgaagatggcggctttctccgagatgggtgttatgcctgagattgcacaagccgtggaagagatggattggcttctcccaactgatatccaggctgaatcgatccccctcatcctaggaggaggtgatgtccttatggctgcagaaacaggaagtggaaaaactggggctttcagtattccggttattcagatagtgtatgaaactctgaaagaccaacaagaaggcaaaaaaggaaaagcgacaattaaaactggtgcttcagtgctgaacaaatgggagaatgatgagtgtgctcagaagaagatcattgcag

SEQ ID NO: 36 - DNA, 611 bp, Bovine
gcgagtgcgtagtccgcgcggccggatcgtggcgtggacgccggacgttctcccagagggaagtctcgtcctgccggtcgtctcgggcgttttggccgaggtcgttggcatcactgagcgtgaccgtgcctgagagctcagggccccctgcacaccctcttctcggcaggattcagagagaaaatacccatcttcctgggacatggcaggtaagaaagtgctcatcgtctacgcacaccaagagcccaggtctttgaacgggtccctgaaggacgtggcggtggctgaactgagccagcagggctgcagcgtcatcgtttcggacctctatgccatgaactttgagcccagggccacagggaaggatatcactggcaccctctccaaccctggtttcttcaactatggcgtggaggcccacaaggcctacaagaagcagtctctgagcagtgacatcattgaggagcagaagaagcttcaggaggccgacctggtgatattccagttcccgctgtactggttcagcgtgcccgccgtgctgaagggctggatggacagggtgctctgtcagggctttgccttcgacttccccggctcctacgatgacggct

SEQ ID NO: 37 - DNA, 317 bp, Bovine
cagcccccctgcactccgtgctgagcaacgtggaggtcaccctcaacgtgctggccgactcggtcctcatggagcagcccccgctccgcaggcgcaagctggagcacttgatcacagagctggttcaccagagagacgtcaccaggtccctgatcaagagcagggtggacaacgccaagtctttcgagtggctcagccagatgcggttctactttgacccgaagcagaccgacgtgttgcagcaactgtcgattcagatggcgaatgccaagtttaactatggctttgaatacctgggtgttcaggacaaagctggt

SEQ ID NO: 38 - DNA, 959 bp, Bovine
ggccgccaggacacaggtgtcgtgaaaaccaccgttaaacctaagccaaaatgggaaaggagaagacccacatcaacatcgttgtcattgggcacgtagattcagggaagtctaccacgactggccatctgatctacaaatgtggcgggatcgacaagagaacaattgaaaagttcgagaaggaggctgccgagatgggaaagggctccttcaaatatgcctgggtcttggacaaacttaaagctgaacgtgagcgtggtatcaccattgatatctccctgtggaaatttgagaccagcaagtactatgttaccatcattgatgccccaggacacagagacttcatcaaaaacatgattacaggcacatcccaggctgactgtgctgtcctgatcgttgctgctggtgttggtgaatttgaagccggtatctccaagaacgggcagacccgtgagcatgcccttttggcttacaccctgggtgtgaaacaactaattgttggcgttaacaaaatggattccactgagccaccctatagccagaagagatacgaagaaattgttaaggaagtcagcacctatattaagaaaattggctacaaccccgacacagtagcatttgtgccaatttctggctggaatggtgacaacatgctagaaccaagtgctaatatgccatggttcaagggatggaaagtcacccgtaaggacggcaatgccagtggaaccaccctgcttgaagctctggattgcattctgccaccaactcgcccaactgacaaacccttgcgtttgcctctccaggatgtctataaaattggtggtattggtactgtccctgtgggtcgtgtggagactggtgttctcaaacctggcatggtggtcacctttgctccagtcaatgtaacaactgaagtgaagtctgtagaaatgcgccatgaagcattgagtgaagccctt

SEQ ID NO: 39 - DNA, 280 bp, Bovine
tggttcctcacctgcattaaccagccccagttccgggctgtcttgggagaggtgaaactctgtgagaaaatggcccagtttgatgctaaaaagtttgcagagagccagcctaaaaaggacaccccacggaaggagaaaggttctcgagaagagaagctgaagccccaggcagagcggaaggagggcaaagaggagaagaaggcagctgcccccgctcctgaggaggagctggatgaatgtgagcaggcgctggctgccgagccgaaggccaaggatccct

SEQ ID NO: 40 - DNA, 167 bp, Bovine
gaacaaatacgatgacgatggagagggtatcaccttgtttcgtccttcccatctgacgaacaagtttgaagacaagactgtggcatatacagaacagaaaatgaccagtgggaagattaaaagatttattcaggaaaacatttttggtatctgccctcacatgacag

SEQ ID NO: 41 - DNA, 666 bp, Bovine
gggaaagctcacaaaatgtgtgatgcatttgtaggtacctggaaacttgtctccagtgaaaactttgatgattacatgaaagaagtgggcgtgggctttgctaccaggaaagtggctggcatggccaaacccactttgatcatcagtttgaatgggggtgtggtcaccattaaatcagaaagcacctttaaaaatactgagatttccttcaaattgggccaggaatttgatgaaatcactccagatgacaggaaagtcaagagcatcgtaaacttagatgaaggtgctctggtacaagtacaaaactgggatggaaaatcaaccaccataaagagaaaactcgtggatgataagatggtgctcgaatgtgtcatgaatggtgtcactgccaccacagtttatgagagagcataagccaagggatattgaaatggatgacgtttgcatcgaactccatgactttctgctggatacgttgtccaaacatatattgttattttccactaataagcaagaaactgattttcttccagactgattttgatatggttatgttggttaaataaaactttttagatttataaggctatgtaatcatttattcattatgtttaacaatttcttactcataattagtgatggaaatataaattgtattattgcttt

SEQ ID NO: 42 - DNA, 559 bp, Bovine
cgcgttctctgtcgtctttcccaacctagcccagcttcaccatggtggacgccttcgtgggtacctggaagttagtggacagcaagaatttcgatgactacatgaagtcactcggtgtcggttttgctaccaggcaggtgggcaatatgaccaagcctaccacaatcatcgaagtgaatggggacacagtcatcataaaaacacaaagcaccttcaagaacacagagatcagcttcaagctgggagtcgagttcgatgagaccacagcagatgacaggaaagtcaagtccatcgtgacgctggatggcggcaaacttgtccacgtgcagaaatggaatggacaagagacatcacttgtgcgggaaatggtggacggaaacttcattctgacactcacccatggcactgcaagttgcactcgtacttacgagaacagcatgactgcctctcttcactgactgtctctgcagtggctactctgactcagacagatgctattttctcttgcatttgataatcactgactggaatctctggtcagtgacagctgatcagtcgt

SEQ ID NO: 43 - DNA, 931 bp, Bovine
cggagagtcgccgcggtttcctgcttcaacagtgcttgaacggaacccggctgctcgtcccccgctaccccggccggccactcagagccagccctcgtcaccacttgacagcgccctccgaccggcccaaggtccccgccaccgctccagtgccgctcggccgtcgccgccaccaccaccaccaccgcccgttttcagccgcccaccatgacgaccgcatccccctcgcaggtgcgccagaactaccaccaggactcggaggccgccatcaaccgccagatcaacctggagctctacgcctcctatgtctacctgtccatgtcgtactattttgaccgtgatgatgtggctttgaagaactttgccaaatactttcttcaccaatctcatgaggagagggaacatgctgagagactgatgaagctgcagaaccagcgaggcggccgaatcttccttcaggatatcaagaaaccagaccgtgatgactgggagaatgggctgactgcaatggaatgtgcgctgtgcttggagagaagtgtgaatcagtcactactggaactgcacaaactggccactgaaaaaaatgatccccatctgtgtgatttcattgagactcattacctgaatgagcaggtggaagccatcaaagaattgggtgaccacataaccaacctgcgcaagatgggggctcctggatctggcatggcagagtacctctttgacaagcacaccctgggacacagtgagagctaagcctcaggctggtttcccacagccacaggggtgacttccctggtcaccaaggcagtgcatgcatatttgggttaccttcatcttttctataagttgtaacaaaacatctacttaagttctttctttagtaccattccttcaaataaagtaatttggtacccaaaaaaaaaaaaaaaaa

SEQ ID NO: 44 - DNA, 610 bp, Bovine
cgtacaaaattgatgcttatgtcccgaaatgaagaggccactaagcatttagaatgcacaaagcaacttgcagcagcttttcatgaggaatttgttgtgagagaagatttgatgggcctggcgataggaacacatggtagtaacatacagcaagctagaaaggttcctggagttacagctattgagctagatgaagatactggaacatttagaatctacggagagagtgctgatgctgtaaaaaaggctagaggtttcttggaatttgtggaggattttattcaggttcctaggaatcttgttggaaaagtaattgggaaaaatggcaaagttattcaagaaatagtagacaaatctggtgtggttcgggtgagaattgaaggagacaatgaaaataaactacctagagaagatggaatggttccatttgtatttgttggcactaaagaaaagccttgggaaatgtgcaagtgcttttcagagtatcatattgcttatctaaaggaagtacaacagctaagaatggaaccgccttccagattgatggaacaacttcgaccagatttggtatgggctttcagaccctttttcccccccagaggggccttga

SEQ ID NO: 45 - DNA, 344 bp, Bovine
gcagatcgcaacatgactctggaagaacttcgtggccaggacacggttccagaaagcacagccaggatgcagggcgccgggaaagcgttgcacgagctgctgttgtcggcgcagcgccaaggctgcctcacggccggcgtctacgagtcagccaaagtcctgaacgtggaccccgacaatgtgaccttctgcgtgctagccgctgacgaggaggacgagggcgatatcgcgctgcagatccacttcactttgatccaagcgttctgctgtgagaacgacatagacatcgtgcgcgtgggcgacgtgcagcggctggcggcgatcgtgggtaccggcgacgaatc

SEQ ID NO: 46 - DNA, 365 bp, Bovine
cttggttgaagagaactcctggctaagcacaaaagcttgccatggaaagaagtgctgcggctggaggaggtacaggccaaactggggatcagtctggaagaaatgcttttgatcacagaggacgcccttcaccctgaaccctacagccctgaggagatctgcaaatgtctgggaattagcctgcaggaactcaagacccaaattctcagtccaaacactcaagatgttctcaccttcaaactctaccagcgggcaaagcacgtgtacagtgaggctgcgagagtgctccagtttaagaagatatgtgaagaggcacctgacaacgtggtccagctgctgggggaactaatgaaccagagccacag

SEQ ID NO: 47 - DNA, 684 bp, Bovine
acgagccgcggtggaagcgggtgcgcgggtcgcctctctgagttatccagttccatccttgtcgctgcggcgacacccgcattctccgtcgccatgactgaacagatgacccttcgtggcaccctcaagggccacaacggctgggtgacccagatcgctaccactccccagttcccggacatgatattgtccgcctctcgagataagaccatcattatgtggaagctgaccagagatgagaccaactatggtatcccacagcgtgctcttcggggtcactcccactttgttagtgatgtggtcatttcctcagatggccaatttgccctctcaggctcctgggatggaacccttcgcctttgggatctcacaacgggcaccaccactcgccgatttgtaggccataccaaagatgtgctgagtgtggccttctcttctgacaaccggcaaattgtctctggctcccgagacaaaaccatcaaactatggaatactctgggtgtatgcaagtatactgtccaggatgaaagccattcagagtgggtgtcttgtgtccgcttctcgcccaacagcagcaatcccattattgtttcctgtggctgggacaagctggtcaaggtatggaacttggcaaattgtaaagctgaagaccaatcacatcggccacacaggctacctgaacac

SEQ ID NO: 48 - DNA, 924 bp, Bovine
gctctcagcagcatggtgaccgtgcccggcagcacctcaggacagaccttcacctgcaacgtagcccacccggccagcagcaccaaggtggacaaggctgttgatcccacatgcaaaccatcaccctgtgactgttgcccaccccctgagctccccggaggaccctctgtcttcatcttcccaccgaaacccaaggacaccctcacaatctcgggaacgcccgaggtcacgtgtgtggtggtggacgtgggccacgatgaccccgaggtgaagttctcctggttcgtggacaacgtggaggtaaacacagccacgacgaagccgagagaggagcagttcaacagcacctaccgcgtggtcagcgccctgcgcatccagcaccaggactggactggaggaaaggagttcacgtgcaaggtccacaacgaaggcctcccggcccccatcgtgaggaccatctccaggaccaaagggcaggcccgggagccgcaggtgtatgtcctggccccaccccaggaagagctcagcaaaagcacggtcagcctcacctgcatggtcaccagcttctacccagactacatcgccgtggagtggcagagaaatgggcagcctgagtcggaggacaagtacggcacgaccccgccccagctggacgccgacagctcctacttcctgtacagcaagctcagggtggacaggaacagctggcaggaaggagacacctacacgtgtgtggtgatgcacgaggccctgcacaatcactacacgcagaagtccacctctaagtctgcgggtaaatgagcctcacgtccctgcaccagcaagccctcacccagcccaccctcccccgggctccaggtccagccaggacgccctagcccctccctgtgtgcattcctcctgggccgccgtgaataaagcacc

SEQ ID NO: 49 - DNA, 640 bp, Bovine
cgagggtcccgacgacgccaaagacaaccatccctcctggaaaacccacaacccaggagtctgaagttgaaaagacaccctgccagtgttccaaatgcccagaacctctgggaggactgtctgtcttcatcttcccaccgaaacccaaggacaccctcacaatctcgggaacgcccgaggtcacgtgtgtggtggtggacgtgggccaggatgaccccgaggtgcagttctcctggttcgtggacgacgtggaggtgcacacggccaggacgaagccgagagaggagcagttcaacagcacctaccgcgtggtcagcgccctgcgcatccagcaccaggactggctgcagggaaaggagttcaagtgcaaggtcaacaacaaaggcctcccggcccccattgtgaggaccatctccaggaccaaagggcaggcccgggagccgcaggtgtatgtcctggccccaccccgggaagagctcagcaaaagcacgctcagcctcacctgcctgatcaccggtttctacccagaagagatagacgtggagtggcagagaaatgggcagcctgagtcggaggacaagtaccacacgaccgcaccccaactggatgctgacggcttcctactttctgtacaagaagg

SEQ ID NO: 50 - DNA, 396 bp, Bovine
tcaacacacagcacgagactgtcacatacctgccagggcacaagctgccccccaatgtggtggctgtccctgatgtggtccaggctgcagcggatgccgacatcctgatctttgtggtgccccatcagttcatcggcaagatctgtgatcagctcaagggccacctgaaggccgacaccattggcgtgtctcttattaagggggtagacgaaggccccaaggggctgaagctcatctctgaagtgattggggagcgccttggcattcccatgagcgtgctgatgggggccaacattgccaacgaggtggctgatgagacgttctgtgagacaaccattggtagcaagaaccaggctcatggacagcttctgaaagagttgatgcagacacccaatt

SEQ ID NO: 51 - DNA, 635 bp, Bovine
ccagcgctcccgccagccttcccgcagcagacacagaccctccgagctgagacccatggcccgagccgcgaccgccgccgccccccggctcctccgcacagcgatgctgctcctgctcctggtggccgccggccggcgcgcagcaggtgagacccggcgcccgggatcccctgggccgtacggggacgggtgggtacccctggggacagcccctaacccactctgtccttcccgcaggggcgcccgtggtcaacgaactgcgctgccattgcctgcaaactttgcaggggattcacctcaaaaacatacagagcgtgaaggtgacgccccccggcccccactgtggccaaaccgaagtcatgtaagtagagccactgttgttgtccttatcacccctgtcgtccggatgccccaacctagacttacagcccgacctcctgtctcacatggattctcccttctctctgcagagccactcttaagaatggtcaggaagcttgtctcaaccctgaagcttccatggttaagaaaatcatcaataagatgcctaacaagtaagtcatggattgtattcctacttgcaactagagccattgctctcaaatactggcatctacctcctgaaaatagtattt

SEQ ID NO: 52 - DNA, 519 bp, Bovine
gcacatctctctggctgacctggtagccatcacggagctgatgcaccccgtgggtgccggctgccaagtcttcaaaggccgacccaagctggccgcatggcgccagcgcgtggaggcggcggtgggggaggtcctcttccaagaggcccatgaggtcatcctgaaggccaaggactctcagcctgcagaccccaccttaaagcagaagatgctgcccaaagtgctggccatgatccagtgagccaggaagcttcgcctctgctctgcccttggcagttcgcagagcaacttcatttccattgtcccgtgggaggcagacccggagagcaggcatggcttgcctgcctgagtcctccgctcctggggccaggttcccacccatctgtcgctggggctgcaaagccacaaagagaatggcacacacagaccttgtctcctttcatctgcgttttctttccagtctgggaaataaacctgggctcagcctgagcctttgcttctaaaaaaaaaaaaaaaaaa

SEQ ID NO: 53 - DNA, 507 bp, Bovine
gcggcagcgggaccacactgaccgtcctgggtcagcccaagtccgcaccctcggtcaccctgttcccgccctccaaggaggagctcgacaccaacaaggccaccctggtgtgtctcatcagcgacttctacccgggtagcgtgaccgtggtctggaaggcagacggcagcaccatcacccgcgacgtgaagaccacccggccctccaaacagagcaacagcaagtacgcggccagcagctacctgagcctgacagacagcgactggaaatcgaaaggcagttacagctgcgaggtcacgcacgacgggagcaccgtgacgaagacagtgaagccctcagagtgtccttagggccctggacccccaccctcgggggccctctggcccacaccccctcccccagctctccatggacccctgagcccccgcccaggtcgcctcacaccaggggcctctcctccctccctgttcctgctttcctgaataaagaccttctcatttatcaagc

SEQ ID NO: 54 - DNA, 658 bp, Bovine
ctcgggggtccccgaccgattctccggctccaagtctggcgacacagccaccctgaccatcagctcgctccaggctgaggacgaggcggattatttctgtgggactggtgactacagtatcaatattgttgttttcggcagcgggaccacactgaccgtcctgggtcagcccaagtccgcaccctcggtcaccctgttcccgccctccaaggaggagctcgacaccaacaaggccaccctggtgtgtctcatcagcgacttctacccgggtagcgtgaccgtggtctggaaggcagacggcagcaccatcacccgcgacgtgaagaccacccggccctccaaacagagcaacagcaagtacgcggccagcagctacctgagcctgacagacagcgactggaaatcgaaaggcagttacagctgcgaggtcacgcacgacgggagcaccgtgacgaagacagtgaagccctcagagtgtccttagggccctggacccccaccctcgggggccctctggcccacaccccctcccccacctctccatggacccctgagcccctacccaggtcgcctcacaccaggggcctctcctccctccctgttcctgcttctcctgaataaagaccttctcatttatcagccgaaaaaaaaaaaaaaa

SEQ ID NO: 55 - DNA, 409 bp, Bovine
cgagcgaccggagactttgattcgaagcccagttgggcggaccaggtggaagaggaaggagaggacgacaaatgtgtcaccagcgagctcctcaaggggatccccctggccactggggataccagtccagagcctgagctactgccgggagctccactgccgcctcccaaggaggtcatcaatggaaacatcaagacagtgacggagtataagatagatgaggatggcaagaagttcaagattgtccgcaccttcagaattgagacccggaaggcctcaaaggctgtggcaaggaggaagaactggaagaagtttgggaactcagaatttgacccaccggggcccaacgtagctaccaccacagtcagcgatgatgtatccatgacattcatcaccagcaaagaggatc

SEQ ID NO: 56 - DNA, 789 bp, Bovine
gcggggatgttgtgctgacccagactcccctctccctgtctgtcgcccctggagagacggtcaccgtctcctgcaagtctactcagagtgtgaaaaacagtaatggaaacacgtatgtgcaatggtttcaacataaagcaggccagtctccacggctattgatctatcgtatttccaatcgttacactggggtcccagacaggttcactggcagtgggtcagagacggatttcacacttacaatcagcaatgtgcaggctgaggatgctggagtctattactgtcttcaaagtacatatactccccatactttcggccaaggaaccaaggtagagatcaaagggtctgatgctgagccatccgtcttcctcttcaaaccatctgatgagcagctgaagaccggaactgtctctgtcgtgtgcttggtgaatgatttctaccccaaagatatcaatgtcaagtggaaagtggatggggttactcagagcagcagcaacttccaaaacagtttcacagaccaggacagcaagaaaagcacctacagcctcagcagcatcctgacactgcccagctcagagtaccaaagccatgacgcctatacgtgtgaggtcagccacaagagcctgactaccaccctcgtcaagagcttcagtaagaacgagtgttagagcaagaggtctacaggctccccagtcgctgtgctgattcggtcccagcccctcacccctcctcaggccctttgtccacagatcaacccctattgcaatcttctgacccatctccccacct

SEQ ID NO: 57 - DNA, 726 bp, Bovine
gtggatatgtgagctggtaccaactgaccccaggatcggcccccagaaccctcatgtatggtgacaccggtctagcctcgggggtccccgaccgattctccgactccaggtctgggaacacagccaccctgaccatcaactcgctccaggctgaggacgaggcagattatttctgtgcatctgctgaagagagtagcagtaaggttcttttcggcagcgggaccacagtgaccgtcctgggtcagcccaagtccccaccctcggtcaccctgttcccgccctccacggaggagctcaacggcaacaaggccaccctggtgtgtctcatcagcgacttctacccgggtagcgtgaccgtggtctggaaggcagacggcagcaccatcacccgcaacgtggagaccacccgggcctccaaacagagcaacagcaagtacgcggccagcagctacctgagcctgacgagcagcgactggaaatcgaaaggcagttacagctgcgaggtcacgcacgaggggagcaccgtgacgaagacagtgaagccctcagagtgttcttagggccctggacccccaccctcgggggccctctggcccacaccccctcccccacctctccatggacccctgagcccctacccaggtcgcctcacaccaggggcctctcctccctccctgttcctgtttctcctgaataaagaccttctcatttaaaaaaaaaaaaaaa

SEQ ID NO: 58 - DNA, 349 bp, Bovine
ctcttaaggaaaaattgattgcaccagttgcagaagaagagacaaggatcccaaacaataagatcactgtagtgggtgttggacaagttggtatggcatgtgccatcagcattctgggaaagtctctgactgacgagcttgctcttgtggatgttttggaagataaactcaaaggagaaatgatggacctgcagcacgggagcttattccttcagacaccaaaaattgtggcagacaaagattactctgtcactgccaattccaagatcgtggtggtaactgcaggagttcgccagcaagaaggggagagtcgcctgaatttggtgcaaaggaacgttaacgtcttcaa

SEQ ID NO: 59 - DNA, 490 bp, Bovine
cgccttgggttcagcgggtctactgttctgtccgcgctcgcgcctggtgccctgcatctcctacagagggacatcccccgagatggagagcaaggccctacttctgctggctctgagcgtgtgcctgcagagtctgaccgtctcccgcggagggctggtcgccgcagacaggattacaggaggaaaagattttagagacattgaaagtaaatttgctctcaggactcccgaagacacagctgaggacacttgccacctcattcctggagtgaccgaatctgtggctaactgtcacttcaaccacagcagcaaaacctttgtggggatccatggctggacggtgacaggaatgtatgagaagttgggtgccaaaactcgtggctgccttgtacaaagaaggaaccggactccaaacgtcattcgttggtgggactggccttgcaacggggcccaagcaagcatttattacagatgtcttgcaagggtacac

SEQ ID NO: 60 - DNA, 433 bp, Bovine
ttgtcattcaccaaggctgaaaactcaaagcaaaataaaccatgaggctgtctgtgactgccctgctgggtactctggccctttgctactacaaggccaatgcaattgtctgtccaacgtttgctgcggatctgacagagttcttctactttcctgacctgctgtacaggctgtcacttgccaagtacaatgcacctccagaagccgtggctgccaagatggaagtgaagcaatgcacggatagattctcagtcaaaaacagattaatcattaccaacatactggggaaaatactgctgaattgtactgtcacagatgtgaaagctgtactaaatccttcttctgcataatcacctgatcttccattgaaaatgtagaggtttcaacatcttgctcaataaatgatttaccctgcaaaaaaaaaaaaaaaaaa

SEQ ID NO: 61 - DNA, 465 bp, Bovine
atcacctgctctggaaccagcagcaatgttggagatggcgattatgtgagttggttccaacagatcccaggatcgggccccagaacagtcatctttggtgcgactcagcgaccctctggggtctccgagcgattctccggctccaggtctggcaacacagccaccctaaccatcagctcgctccaggccgaggacgaggcggattatttctgttcatctcccgacaccactaacaatgttgctttcggcagcgggaccaccctgagcgtcctgagacagcgactggaaatcgaaaggcagttacagctgcgaggtcacgcacgacgggagcaccgtgacgaagacagtgaagccctcagagtgtccttagggccctggacccccaccctcgggggccctctggcccacaccccctcccccacctctccatggacccctgagcccctacccaggtcgcctacacca

SEQ ID NO: 62 - DNA, 308 bp, Bovine
ctcgtttatgattttgccaattttggtgttctgaggttatcggagccagcaccactctttgacctggccatgctcgccttagacagtcccgagagtggctggacagaggaggatggtcccaaggaaggacttgctgagtacattgttgagtttctgaaaaagaaggctgagatgcttgcagactacttctctctggagattgatgaggaagggaacctggttggattaccccttctgatcgacaactatgtgcccccgctggagggtctgcctatcttcatcctccgactggccacagaggtgaactg

SEQ ID NO: 63 - DNA, 495 bp, Bovine
atttcttatcaagtgggttggctgattccggtcttctgttaccggatttttgactttgttctcagctgcctggttgctatcagttctctgacttacttgccaagaatcaaagaatatctggatcagttacctgattttccctataaagatgacctcctggcattggactccagctgcctcttgttcattgttcttgtgttctttgccttgttcatcatttttaaggcttatctgattaactgtgtttggaactgttataaatacatcaacaacagaaacatgccggagattgctgtgtacccagcctttgaggcgcctccacagtatgttttgccaacctatgaaatggcagtgaagatgcctgagaaagaaccaccacctccttacatacctgcctgaagaaattctgcctttttcaataaaccctataccagctttttgtcttggtcattttacagaatgctgcaacacagggctcatcatacttggttgatc

SEQ ID NO: 64 - DNA, 826 bp, Bovine
gctcccatcggggtcttcaccatccccccatccttcgccgacatcttcctcacgaagtcagccaagctgtcctgtctggtcacaaacctggcctcctatgatggcctgaacatcagctggtcccgtcagaacggcaaggccctggagacccacacttattttgggagacacctcaacgacaccttcagcgcccggggtgaggcctcggtctgctcggaggactgggagtccggagaggagttcacgtgcacagtggcccactcggacctgcccttcccagaaaagaacagcgtctccaagcccaaagacgtcgccatgaaaccgccgtccgtgtacctgctgcctccaacgcgggaacagctgagcctgcgggagtcggcctccgtcacctgcctggtgaagggcttcgcgcccgcggacgtgttcgtgcagtggctgcagaggggggagcccgtgaccaagagcaagtacgtgaccagcagcccggcgcccgagcctcaggaccccagcgtgtactttgtgcacagcatcctgacggtggccgaggaggactggagcaaaggggagacctacacctgcgtcgtgggccacgaggccctgccccacatggtcaccgagcggaccgtggacaagtccaccggtaaacccaccctgtacaacgtgtccctggtcctgtctgacacagccagcacctgctactgatgcctggtcagagcccccgggtgaccgtcgctgtgtgtgcatgagtgcagactaaccgtgtcggtgcgcgagatgctgcactctataaaaattagaaataaaaagatccattcaaagctgaaaa

SEQ ID NO: 65 - DNA, 745 bp, Bovine
cccggccctggcccgggcccggggagcaacttgacttcggccccagggccctccaccacaacacgctcgctgaccgcatgccctgaggagtccccgctgctcgtcggccccatgctgattgagtttaacatacctgtggacctgaagcttgtggagcaccagaacccgaaggtgaagttgggtggtcgctacacccccacggactgcatctctcctcacaaggtggccatcatcattccattccgcaaccggcaggaacacctcaagtactggctgtattacttgcacccaatcctacagcgtcagcagttagactatggcatctatgttatcaaccaggctggagagtccatgttcaaccgcgcaaagctcctcaatgttggctttaaagaggccttgaaggactatgactacaactgctttgtgtttagcgatgtggacctcatcccaatgaacgaccataacacctacaggtgcttttcacagccacggcacatttctgtagcaatggataagtttggatttagcctaccttacgtgcagtattttggaggtgtctctgctctaagtaaacaacagtttctcagcatcaatggatttcctaataactactggggctggggaggtgaagatgatgacatttataacagattagactttaaaggcatgtctgtgtctcgcccaaatgctgtgatcgggaagtgtcggatgatccgcactcgagagacaaagaaaaatgaaccta

SEQ ID NO: 66 - DNA, 897 bp, Bovine
gcctgcgcggctgctggttccaatagtagtcgtttaattccgtccggcttctctcccacataagtgcgtgcagccaacccatggaggattcaatggacatggacatgagccccttgaggccccagaactatcttttcggttgtgaactaaaggctgacagagattatcacttcaaggtggataatgatgaaaatgagcaccagttatctttaagaacggtcagtttaggggctggagcaaaggatgagttacatgttgttgaagcagaggcgatgaattatgaaggcagtccaattaaagtaacactggcaactttgaaaatgtctgtacagccaacggtttctcttgggggctttgaaattacaccacctgtggtcttacggttgaagtgtggttcagggcctgtgcatatcagtggacagcacttagtagccgtggaggaagatgcagagtcagaagaggaggaggaggaggaggtgaaactcctgagtatatctggaaagcgttctgcccctggaagtggtagcaaggttccccagaaaaaagtgaagcttgctgctgatgaagatgaagatgatgatgacgatgacgatgatgatgatgatgaagatgatgatgatgacgattttgatgaggaagttgaagaaaaagctccagtaaagaaatctgtacgagatactccagccaaaaatgcacaaaaatcgaaccaaaatggaaaagactcaaaaccgtcaacaccaagatcaaaaggtcaagaatccttcaaaaaacaggaaaaaacaccgaaaacacctaaaggacctagctctgtagaagacattaaagcaaaaatgcaagcaagtatagaaaaaggtggttcccttcccaaagtggaagccaagtttatcaattatgtga

SEQ ID NO: 67 - DNA, 372 bp, Bovine
gagcaatccagaagaaaaagaagaaagcaggaggcataacctgtccagacttcaaatactacaaagctacagtaatccaaatagcttggtactggcacaaaagcaggcatgtggatcaatgaatcagagcagagagcccagaaataagcccacacacctacagtcagtcagtctttgacagaacagacaaggatctacaatggagaaacgatggtctctttagcaagcggtgctgggaaagttgggcgtgcatgtgtgctcagtcactaagtttagctgcatataaatcaataaagttagacacagcctcacaccatacacaaaaataaactcaaaatgagttaaagacttaagcataagacataacaccaa

SEQ ID NO: 68 - DNA, 545 bp, Bovine
gagaagttaaaagaggcaccagaaggaactttcttgattagagatagttcgcattcagactacctactaacaatatctgttaagacatcagctggaccaactaatcttcgcatcgaataccaagatgggaaatttagattggactctatcatatgtgtcaagtccaagcttaaacaatttgacagtgtggttcatctgatcgactactatgttcagatgtgcaaggataagcggacaggcccagaagccccccggaacggcactgttcacctttatctgaccaagccactctacacatcagcaccacccctgcagcatctctgtagactcaccattaacaagtgtaccagcaccgtctggggactgcctttaccaacaagactaaaagattatttggaagaatataaattccaggtataagtgtttcttttttttctaaacatgcctcctatagaatatctccgaatgcagctatgtaaaagagaaccaaatcttgagtgacggctctggataacctacgcggaattctaagaccagcttgaagt

SEQ ID NO: 69 - DNA, 770 bp, Bovine
gggagactggtgttctcaaacctggcatggtggtcacctttgctccagtcaatgtaacaactgaagtgaagtctgtaaaaatgcaccatgaagcattgagtgaagcccttcctggggacaatgtgggctttaatgtcaaaaacgtgtcggtcaaagatgtccgtcgtggcaatgtggctggtgacagcaaaaatgatccacccatggaagctgctggcttcacagctcaggtgattattttgaaccatccaggccaaatcagtgctggatatgcacctgtgctggattgtcacacagctcacattgcttgcaagtttgctgagctgaaggagaagattgatcgtcgttctgggaaaaagctggaagatggccctaaattcttgaaatctggtgacgctgccatcgttgatatggttcctggcaagcccatgtgtgtcgagagcttctctgattatcctcccctgggccgttttgctgtgcgtgacatgagacagacagtcgctgtgggtgtcatcaaagcagtggacaagaaggcagctggagctggcaaggtcaccaagtctgcccagaaagctcagaaggctaaatgaatattatccccaatacctgccaccccagtcttaatcagtggtggaagaacggtctcagaactgtttgtctcaattggccatttaagtttaatagtaaaagactggttaatgataacaatgcatcgtaaaaccttcagaaggaaaggagaatgtttttgtggaccatatgttttgtgtgtg

SEQ ID NO: 70 - DNA, 591 bp, Bovine
ggacctcgacgccctggtgcagttcctgtccatcggcaccctgctggcctacaccttcatggccatcagcgtgcttgtgctgcggttccagacggcctctcagtcccgctcgcccagcctggccggctccggcccgaaggccaaggagtacagctccttctctgaccacttggagctggtgggcgcagggcacggccccgagccggggcggctgcggccagccctgaggccctacctgggcttcctggacaggggcagccccggcgcggccgtgcgcggggccgtctgcgggctggtggtctccgccatcgccctgggctgcgtgctgatgctcgggcactcggtcctacgcctccccctctggggcttcctcctgctgctgctgtgcagcagcgtcacgtttctgctcagtctcctcgtcctgggggcccaccagcagcaacgcctgaaggacaccttccagatgcccctggtgcccctgattccagctctgagcatcgtcctcaacttctgcctcatgctgaagctgagctacctgacctgggtgcgcttcaccatctggctgctgataggactcttggtgtattttcg

SEQ ID NO: 71 - DNA, 373 bp, Bovine
ttctacgtgtcacagcctggaagttcagtggtcacttctctttccccaggagaagctgtaaagaaacacattggtttgctgcgtattaaaggaaggaagatgaatatgcagaaaattcctctccgcacagtgaggcagtttttcatggaagatgttgttctggctgatcatccagacatttttaacccagataatcctaaagtaacacaagtcatacagaacttctgcttggagaaggttgaagaaatgcttgaaaatgcagaacgggaacgtctgggaaattctcaacagccagagaagcctcttatacgactgcgagtggactatagtggaggctttgaaccattcagtgttcttcgctttagccagaaat

SEQ ID NO: 72 - DNA, 344 bp, Bovine
atcctaccatgttacaggaccctgatgtcagagagttcttggaaaaagaagagctgccacgtgctgtgggtacccagacattgagtggcgctggtctcctcaagatgttcaacaaagctacagatgcggtcagcaaaatgaccatcaagatgaatgaatctgacatttggtttgaggagaagctccaggaggtagagtgtgaggagcagcgcttacggaaactgcatgccgttgtagaaactctagtcaaccacaggaaagagctagcgctaaacacagcccagtttgccaagagtctcgccatgcttgggagctctgaggacaacacagcactgtcacgggct

SEQ ID NO: 73 - DNA, 531 bp, Bovine
cagattagcagacttgaagaaagagaagcggaactgaagaaagaatataatgctttgcatcagagacacactggatgatccataattatatggaacacttagaaagaacaaaacttcatcagatctcagggagtgatcaactagaatccacagctcatagtagaattagaaaagaacgtcctatatcgttagggattttccctttaccttctggagacggattgcttacgcctgacactcagaaaggtggcgagacccctggatcagaacaatggaaatttcaggaattaagtcaaccacgttctcataccagtctgaaggatgaactttccgatgttagccagggaggatctaaagccaccactccagcgtcgacagctgcttcagatgtggcagcaacacctagcgatactcccttacatgaggagaacggaggggttgtggaggttgcagatacacccgataagtcagagataagcaagcatatctccatcccattgacagaaacgaataaaacatcaggagcatcgg

SEQ ID NO: 74 - DNA, 658 bp, Bovine
agcccttctttttgtcccaagacgagctccttttgacctgtttgaaaacagaaagaagaagaacaacattaagttgtatgttcgcagagtattcatcatggataactgcgaggagctaatccctgaatacctgaatttcattagaggtgtggtggattctgaggatcttcctctgaacatttcccgtgagatgttgcaacaaagcaaaattttgaaagttatcaggaagaatttggtcaaaaagtgcttggaactcttcactgaactggcagaagataaggagaactacaagaagttttatgagcagttctctaaaaatattaagcttggaatacatgaagattctcaaaatcggaagaagctttcagagctgttgaggtactatacttctgcttctggtgatgagatggtttctctcaaggactattgcacaagaatgaaggaaaaccagaaacacatctattacatcacaggtgagaccaaggaccaggtggccaactcggccttcgtggagcgcctccggaagcacggcttggaagtgatctacatgatcgagcccattgatgagtactgtgtgcagcagctgaaggagtttgaggggaagaccttagtgtcagtcaccaaagagggcctggaactttcagaagatgaggaagag

SEQ ID NO: 75 - DNA, 615 bp, Bovine
tggaaaccctcgtacgaacggcatgtgttcagtgtgctataaagaacatcttcaaagacagaatagtagtaatggtagaataagcccacctgcgccttctgtcacaagtctgtctgagtccttaccagtccagtgcacagacggtagtgtcccagaggctcagtcagcgctagactcaacagcttcatctgtgcagccaagccctgtgtcaaatcagtcacttttatcagaatcagtagcgtcttcccaagtggacagtacatctgtggacaaagcaatacctgaaacagaagacctgcaagcttcagtatcagaaacggcacagcaggcatctgaagagcaaagcaagtctcttgaaaaaccaaaacagaaaaagaatcgctgtttcatgtgcagaaagaaagtgggacttactgggtttgaatgccggtgtggaaatgtttactgtggtgtacaccgttactcagatgtacacaattgctcttacaattacaaagctgatgctgctgagaaaatcagaaaagaaaatccagtagttgttggtgaaaagatccagaagatttgaactcctgatggaatacaaaatcctttgaccatctgcaaactaaaaactga

SEQ ID NO: 76 - DNA, 214 bp, Bovine
  misc_feature (1)...(214): n = A,T,C or G
gaaacattccagcaggcacaaccgtggacacgaaaatcacccacccaactgagtttgacttctacctgtgtagtcatgctggcatccagggaacaagcaggccctcgcactaccatgtgctctgggatgacaatcgcttctcttccgacgagctgcagatcctcacctaccagctgngtcacacctacgtgcgctgcacacgctccgtggtcat

SEQ ID NO: 77 - DNA, 184 bp, Bovine
  misc_feature (1)...(184): n = A,T,C or G
ctgccctcttggatgtgcaattcagaaacaccaccattgggctgaccgtgttcgccatcaaaaaatacgtggtcttcctgcggctcttcctggagacggcggagaagtacttcatggnggggcacaaggtcatctactacgtcttcaccgaccggccggcggacgtgccccagatcgccctcca

SEQ ID NO: 78 - DNA, 565 bp, Bovine; identical to SEQ ID NO: 8

SEQ ID NO: 79 - DNA, 323 bp, Bovine
ggacttcggcacgatgaaggacaagatcgcagcgaacgagtacaagtcagtcacggagttcaaggcagatttcaagctgatgtgtgacaacgcgatgacatacaacaggccagacaccgtgtactacaagttggccaagaagatcctgcacgctggcttcaagatgatgagcaaagagcggctcttagctctgaagcgcagcatgtcgtttatgcaggacatggatttctctcagcaggcggctcttctgggcaacgaagacacggctgccgaggagcctgtccccgaggtcgtgcctgtgcatgtagagacggccaagaagt

SEQ ID NO: 80 - DNA, 450 bp, Bovine
caagatctgaacagcacagccgccccacacccccgcctgtcccagtacaagtccaagtacagttccttggagcagagtgagcggcggcgccagttactggaactgcagaaattaaagcgtctggattatgtgaaccatgccagaagactggctgaagatgactggacggggatggagagtgaagaagaagaagaaaagaaagatgatgaggaaatggacgttgacactggcaaggagttaccaaaacgctatgctaatcaattaatgctgtcagagtggttaattgacgtcccttcagatttggggcaggaatggattgtggtcgtttgccctgttggaaaaagatcccttatcgtggcttcccagggtcttaccagtgcctacaccaggagtggctactgggtcaacacgtttccttcccttctgccaggaggcaacaggcgaaactca

SEQ ID NO: 81 - DNA, 373 bp, Bovine
aatccaggaactacgaagaggatctcaagcagctaatatttattgcattaacttcaatcaggatgcttccctcatctgtgtgtccagtgaccacggcacggtgcacatttttgcagctgaagatccaaaaaggaataaacaatcaagtttggcatcagccagtttccttccaaaatacttcagttccaagtggagtttctccaagtttcaggtcccctcaggctctccatgcatttgtgcctttgggacagagccaaacgctgtcattgcgatctgtgcggacggcagctactacaagtttctgttcaaccccaaaggagagtgcgtccgggacgtgtatgcccagttcctggagatgaccgatgacaagctt

SEQ ID NO: 82 - DNA, 369 bp, Bovine
gggaagtgctgtggcgaagagctcaggtgtgggcaggtcacccgggcagcggagcccttcccaaggtggatgtggaacagcttgtcctctgctagagccgggcctgggctgagcaccagccacaggaccctctggaccctgggactgtggccttgactcctgctacaactcaagtggggctctgcagtttctccagaagaattcctctaagtatcacttcagacgcaccaagatgttgccggttagcggtgggttccacacccgcctcatggagccggccgtggagcccctggtgcaagtgttaaaggcgattgatgtcaagaagcccctggtgtccgtgcactcgaacgtcgatgggaacaaatacat

SEQ ID NO: 83 - DNA, 601 bp, Bovine
acgatacagagaagattagcatggcccctgcgcaaggatgacacgcaaattcgtgaagcgttccatatttttgtgacgtccctgctaaggccattgccagtgccctacatgggctttgtgcccagatcttgtcggagcgagtggaggtcagtggtgattccccttgctgctcactagaccccattacccctgaagacctgcctcgacaagtggagctactggatgctgtgagccaggctgctcagaagtacgaggcactgtacatggggaccctgccagtcaccaaagccatgggcatggatgtgctgaacgaggccattggcagaggctggtgcagaggaggaaccactgtggcagtgtcctgtgcgcctcgtgacctttattggtgttggtcgtgacccacacacctttggtctcattgccgacctgggccatcagagcttccagtgtgcagccttctggtgccagccccatgcagggggactctctgaagctgtgcaggcggcttgcatggttcaataccagaagtgtcttgtggcctctgagcttcgaggcaaggcctggggtgccaagcccgcgcacgcctgcggttaagcggaca

SEQ ID NO: 84 - DNA, 405 bp, Bovine
cgaagactcaggcgcgtgccggccaggcctcccgctcgcctagtccgcgttcctcggcgcccctcgtccgcgcctgccccgcgcctcccgtctgcgcaccccagccgcccgccaggcccccagccgctctccaggccgccaggccccgcgcccgacacccaggacaggcacgcgccccgcaggccgcccgccgcccgcgccgccatgggggtagagggctgcaccaagtgcatcaaatacctgctcttcgtcttcaatttcgtcttctggctggcaggaggtgtgatactgggcgtggccctgtggcttcgccatgacccgcagaccaccaacctcctgtatctggagctcggagaccggcccgcccccaacaccttctatgtgggcatctacattcttatcgcc

SEQ ID NO: 85 - DNA, 361 bp, Bovine
attttcctgggctccaaaatcactgcagatggtgactgcagccatgaaattgaaaggtgctttctccttggaagaaaacttatgaccaacctagacagcatattaaaaagcagagacattactttgccaacaaaggtccatccagttgaagctatggtttttccagtagtcatgtatggatgtgagagttggaccataaagaaagctgagtaccgaagaattgactcttgagagtcccttggactgcagtggtacctagcagatttgcgtgatatgttagtatgataatgaaatgctaagctaaatgctctgagaagggtgatccaaagattagacactctctctcttcctcattctgatg

SEQ ID NO: 86 - DNA, 918 bp, Bovine
gagaagcgcagcggaggttttgctggtttcggaccccagcggccggatggtgaaatcctccctgcagcggatcctcaacagccactgcttcgccagagagaaggaaggggataaacccagcgccaccgtccacgccacccgcaccatgccgctccttagcctgcacagccgcggaggccgcagcagtgagagttccagggtctccatcaactgctgtagtaacctgggtccagggcctcggtggtgctcctgatgtccctcacccacccctgaagatcccaggtgggcgagggaatagtcagagggatcacaatctttcagctaatttattttactctgataatcggctgaatgtaacagaggaactaacgtctaataacaagacgaggatttttaatgtccagtccaggctcacagaggccaaacatattaactggagagcggtgctgagcaacagctgcctctacgtcgagatcccgggcggcgctctgcccgaggggagcaaagacagcttcgcagttcttcttgagtttgctgaagagcagctccatgttgaccacgtcttcatttgcttccacaagaaccgtgatgatcgagccgccttgctccgtaccttcagctttttgggctttgagattgtgagaccggggcatccccttgtccccaagagacccgacgcttgcttcatggcctacacgtttgagagagagtcctcgggtgaggaggagtagtgtgacgaccctggggccactcaggggcctcagttgcagtcccttgcgtgtgtgctttgcgccgtgcctggtgcgggtggtgtgatcaactgcgctgaccagcgtcagcctgctcacctgctggtttgtccgcatgttgtaattgtgcaaataaacgctcactccaaaaaaaaaaaaaaaaaaaaaa

SEQ ID NO: 87 - DNA, 584 bp, Bovine
cgccgcagcccgacccggctcctcggtggagagaagatggtgggccggaacagcgccatcgccgccggcgtgtgcggggcccttttcatcggttactgcatttacttcgacccgcaagagacggagtgaccccaacttcaagaacaggctgcgagaacgaagaaagaaacagaagcttgccaaggagagagctgggctttccaagttacctgaccttaaagatgctgaagccgttcagaaattctttctagaagaaatacagcttggtgaagaattactagctcaaggtgaatatgagaagggtgtggaccatctgacaaatgcgattgctgtgtgtggacagccacagcagttactgcaagtattgcaacaaactcttccgccaccagtgttccagatgcttctgactaagctcccaacaattagtcagagaattgtaagtgctcagagcttggctgaaagatgatgtggaatgagaaacaaatgtcaacatactgatctcaattaaaacatatttttaaaatcttatcttaaaagatgatcggctctttgggagtaaaggcaattaagcttggttcgggctg

SEQ ID NO: 88 - DNA, 456 bp, Bovine
cttaaagtgccatgagaaaatttgcatactgcaaggtggtcctagccacctccttgatttgggtactcttggatatgttcctgctgctttacttcagtgaatgcaacaaatgtgatgaaaaaaaagagagaggacttcctgctggggatgttctagagccagtacaaaagcctcatgaaggtcctggagaaatggggaaaccagtcgtcattcctaaagaggatcaagaaaagatgaaagagatgtttaaaatcaatcagttcaatttaatggcaagtgagatgattgcactcaacagatctctaccagatgttagattagaagggtgtaaaacaaaggtgtatccagataaccttcctacaaccagtgtggtgattgttttccacaatgaggcttggagcacacttctgcgaactgtccatagcgtcattaatcgctcaccaaagcacatgctag

SEQ ID NO: 89 - DNA, 381 bp, Bovine
cgggtgcccggccgccacggctacgccgccgagttctccccgtacctgccgggccgactggcctgcgccgcctcgcagcactacggcatcgcgggcagtggaactctgctaatattggatcaaaatgaatctgggcttaggctttttagaagctttgactggaatgacggtttgtttgacgtgacctggagtgagaacaatgagcatgtgctggtcacctgcagtggcgacggctccttacagctctgggataccgccagggccacagggccgctgcaggtcttcaaggagcacactcaggaggtgtacagtgttgattggagccaaaccagaggtgaacagcttgtggtgtctggctcatgggatcaaactgtcaaactg

SEQ ID NO: 90 - DNA, 886 bp, Bovine
tacgctgtgtgacatggatgaatctgcattgacccttggcacgatagatgtatcttatttgccaaattcttctgaatacagcattggtcggtgtaagcatgccaccgaggaatggggtgagtgtggttccaggcctactgtcttcagatctgcaaccttgaagtggaaagaaagcctaatgagccggaagaggccctttgtgggaaggtgctgttattcctgtactcctcagagctgggataaatttttcaaccccagtatcccgtctttgggtttgcggaatgttatttatatcaatgaaacgcatacaaggtaaaaataaatttcttatatttctaatgcatgtcaagagaaagactccataataaccaaccagtgtactttaagatgaatctctttatattttttttgaagagttaatagggtattaacttctaacatataatactttatctaaaaattggaacctgagagacctattactattctcctgctgtattatataatattttcatgagactatagatattacttagaggtttctttaacttatttatataatgatgttgctgctgaaatgtgccaaaatgtgatagaaatttgtcggaggagaggtgtaggtttgaatgtcttatgttatattattattttatacttaaaaaagtatcactttctgaaatctgcctgatctatagagtgagtattctttgatttattactggcttaagtctttttttagaattcttacttccaaattctaggcatcagagtgagagaaaaagaggctttattctcagtggatttaggctgggggatgaggaaggagggtgaatgtctgcaagacaagagcagaatgggagccaaaaccactaccagtccttgtagt

SEQ ID NO: 91 - DNA, 690 bp, Bovine
ggtaacactctgttactggagccagctcagctcagcggagcagatataaaccgagaagggcaaactcatcgagagggcgtcttcgggaccatggcgaatggatatacatatgaagattatcaagacactgcaaaatggcttttgtctcataccgaacagcgacctcaagtggcagtgatctgtggttctgggttaggaggtctggttaacaaattgactcaagcccagacctttgactacagtgaaataccaaactttccagaaagtacagtgccaggtcatgctggtcgactggtgtttgggatcttgaatggcagagcctgtgtgatgatgcagggcaggttccacatgtatgaaggctatccgttttggaaggtgacattcccagtgagggttttccggcttctgggtgtggagaccctagtggtcaccaacgcagctggagggctcaaccccaactttgaggttggcgatatcatgctgatccgtgatcacatcaacctacctggtttcagtggtgagaaccctctcagagggcccaatgaggaaaggtttggagttcgtttccctgccatgtctgatgcctacgaccgggatatgaggcagaaagctcacagtacctggaaacaaatgggggagcaaagagagttacaggaaggcacctacgtgatgttggggggtcccaatttt

SEQ ID NO: 92 - DNA, 472 bp, Bovine
ccagtccgaacctctgactggagtttttaccacagaggaagttccagcccagcagtacttggaaattgatgaggtgacgccagacagctttcgagtgagctggcacccgctgtcggcggacgaggggcagcacaagctgatgtggatcccagtctacgggggcagcaccgaagaagttgtccttcaagaagaccaagactcatatgttatcgaaggcctggagcctggcactgagtatgaagtctccctgctggctgtactggacgatgggagtgagagtgaggttgtgactgctgttgggaccacacttgacagtttttggacagaaccacctacgaccgaagaagcccctaccagacctgtgacatcagttttccgcaccggaatcaggaacctggtggtagatgctgagaccacttctagcctgcgggtagcctgggacatttcaaacagcagtgtacaagcaattcaa

SEQ ID NO: 93 - DNA, 431 bp, Bovine
agcaatgatgggctatgactagtgaaaaattgcttaaagtaaattaacgttcaaaatgctatactttcaaaggccccggcaacagaccacttcccagaatggaaggcaggaacttctcgccagttccaagcaaacctagatcacaatcccctggggaagaagagaattcattaaatgaagactggtacgtttcttatgttacccgaacagaggcagaagcggctcttagaaagataaaccaggacggcactttcctggttagagacagctctaaaaaaacaatatccaatccatacgttctcatggtgttgtacaaagataaagtttacaacatccaaatccgttatcaggaagaaagccaagtttacttgttgggaactggactccgagggaaagaggactttctgtctgtgtcagatattatcgactac

SEQ ID NO: 94 - DNA, 561 bp, Bovine
gccagcgctaggaaggctgcacaggtgaccatccagtcttcagggacattttccacaaaattccaagtggaaaacagcaaccgcctgttactacagcaggtttcattgccagaagtgcctggagagtactgcatgtcagtcacaggagaaggatgtgtttacctccagacatctttgaaatacaatattctcccgaaaaaagatgagttcccatttgctttggaggtgcagactctgccccaaacttgtgatggacccaaagcccacaccagcttccagatctcactgagtgtcagttatattggaagccgtccagcctccaatatggcaattgttgacgtgaagatggtatctggcttcattcccttgaaaccaacagtgaaaatgctagaaagatctaacgtgagccgaacagaagttagcaacaaccatgttttgatttatctggataaggtgacaaatgagaccctgaccttgaccttcacagttctgcaagacatcccagtaagggatctgaaacctgccatagtgaaagtctatgactattatgagacagatgag

SEQ ID NO: 95 - DNA, 556 bp, Bovine
acgaccacttggaggccaagaaacccttgtccactcccagcctgactactgaggattggcttgtccagaaccatcaagacccatataaagtagaggaggtctgcaaagccaatgagccctgtacaagttttgcagagtgtgtatgtgatgaaaattgtgagaaggaagctctgtgtaaatggcttctgaagaaagaaggaaaggataaaaatggtatgcctgtggatccaaaacctgaacctgggaaacacaaggattccctgaacacgtggctctctccgtccggaagagaggcagcagagcaagctcgggcaccacaggcgacggctgctggagttgctgattccttccaagtcataaggagcagtcccttgtcagagtggctcatgaccccctcacacaaagaaggatgtcccaacaaggaagcgcctcttacagaggacagggccagcaaacaaaagctcacaagccccttggccactgcctggtgtccctttaacacagctgactgggtcttgccagcaaagaagacaggaaatcttagccagttatcctc

SEQ ID NO: 96 - DNA, 487 bp, Bovine
gtgaaagtagaatctctcatgaaaacggaacaatattatgctcaaaaggtagcacctgctatggcctttgggagaaatcaaaaggggacatcaatctggtaaaacaaggatgttggtctcacattggtgatccccaggagtgtcactatgaagaatgtgtagtaactaccaccccaccctcaattcagaatggaacataccggttctgctgctgtagcacagacttgtgtaatgtcaactttactgagaattttccacctccagacacaacaccactcagtccacctcattcatttaaccgagatgagacaataatcattgcattggcatcagtctctgtattagctgttttgatagttgccttatgctttggatataggatgctgacaggagaccgcaaacaaggtcttcacagtatgaatatgatggaggcagcagcatcagagccctctcttgacctaaataatctgaaactgctggagctgat

SEQ ID NO: 97 - DNA, 258 bp, Bovine
gaaaagcgggcatacctccagtccaggttcccccagctcaacgaaaccagctttgccaactcccgggacacatcctttgagcagcatgtgctgtggcacacagccgggaagggtgctgacctggtcctcaactccctggcggaagagaagcttcaggccagtgtgcggtgcctggcccagcacggtcgattcctggaaattggcaaatttgacctttccaaaaaccaccccctgggcgctgggcacccaccctatttg

SEQ ID NO: 98 - DNA, 460 bp, Bovine
tgtaaatgcggccggtggaccaaccccaagccagagaggtctgtctgacctggctttgtgtggaccagcggcgaaccagtgtgcagggccggccaaggacagggtggactgcggctaccccgaggtcacccccgagcagtgcaacaaccgcggctgctgcttcgactccagcatccacggggtgccctggtgcttcaagcccctgcaggaagcagaatgcaccttctgaagccacgtggccgcccgacacccagggaggggatctccgtacttgggtaccctgcccgcccacccagtgctcatccctctgcttctctcaaactgctcctggccaggcctgaaccaaatgcctggggcctgatgtcttaaagaataaagctcccgtgctcagcatgaggacaggtcttcattcctaaaaaaaaaaaaaaaaaaaaaaaaaaaaaaaaaaaaaaaaaaaaaa

SEQ ID NO: 99 - DNA, 234 bp, Bovine
ctcaggaccaacctcagaaaagccagctcgcagtcatccatggacccctgatgattccacagataccaatggatcagataattccatcccaatggcatatcttacactggatcaccaactacagcctctagcaccatgcccaaactccaaagaatctatggcagtgtttgaacagcattgtaagatggcacaggaatacatgaaagtccaaacagaaattgcattgttattaca

SEQ ID NO: 100 - DNA, 377 bp, Bovine
tgcccatcatcctagtgggcaacaaaagcgacctggtgcgctctcgcgaggtctccttggacgagggccgggcctgtgccgttgtctttgactgcaagttcatcgagacgtcggctgcactgcaccacaacgtccaggcactgttcgagggtgtcgtgcgtcagatacgcctgcgcagggacagcaaagaggccaacgcgcgtcggcaagcgggtacccggcggcgagagagcctgggcaagaaggcgaagcgcttcctgggccgcatcgtcgctcgaaacagccgcaagatggccatgcgtgccaagtccaagtcctgccatgacctatcggtgctctagaccctggggcacttgctgtattgtgcaaacagatgg

SEQ ID NO: 101 - DNA, 584 bp, Bovine
ctgttttgtagccagcattcttctccttgctgttgctcgttgcattctgtttctcatcatttggctcataactggaggaaggcaccatttttggttcttgccaaatctgactgcggacgtgggcttcattgactccttcagacctctgtacacacatgaatacaaaggaccaaaagcagacttaaagaaagatgagaaatctgaaaccaaaaagcaacagaagtccgacagtgaggaaaagtcagacagtgagaaaaaggaagatgaggaggggaaagtaggaccaggaaatcacggaacagaaggctcaggtggagaacggcattcagacacagacagtgacaggagggaagatgaccgatcccaacacagtagtggaaatgggaatgattttgaaatgatcacaaaagaggaactggaacagcaaacagatggggattgtgaagaggaggaggaagaagacaacgatggagaaacaactaaatcttcacatgaaaaatcataatctgactaatttggggactaaatatgtgcaagaggttggattttctatgttggctgatcatcataatgtacacatgaca

SEQ ID NO: 102 - DNA, 321 bp, Bovine
ctgtgaaggccctgaagaggaaagtgaggacgaccctcagcttgaaggcagagatcctgatatttggcacgttggttttaagatctcgtgggacatagagacacctggtttggcgataccccttcaccaaggagactgctattttatgcttgatgatctcaatgctacccaccaacactgtgttttggctggtttaccacctcggtttagttccacccaccgagtggcagagtgctcaacagggaccttggagtacatcttacagcgctgccaggtggccctgcaaaatgtccgcgaggaggcagacaacggtgaaatctc

SEQ ID NO: 103 - DNA, 381 bp, Bovine
tgatgctgatggtgctgtgccctcccctggcctgggccagggagatccaaccacatttcctggagtattctacgagcgagtgtcatttcttcaacgggaccgagcgggtgcggttcctggacagatacttccataatggagaagagttcgtgcgcttcgacagcgactggggcgagtaccgggcggtgaccgagctagggcggccggacgccgagtactggaacagccaggagatcctggagcgggcgcgggccgcggtggacacgtactgcagacacaactacgggggcgtggagagtttcactgtgcagcggcgagtggagcctacagtgactgtgtatcctgcaaagactcagcccctgcagcaccacaacctcctgg

SEQ ID NO: 104 - DNA, 512 bp, Bovine
ggcacgagttaacattagcagaataccatgaacaagaagaaatcttcaaactcagattaggtcatcttaaaaaggaggaagcagagatccaggcagagctggaaaggctagaaagggttagaaatctacatatcagggaactaaaaaggatacataatgaagataattcacaatttaaagatcatccaacgctaaatgacagatatttgttgttacatcttttgggtagaggaggtttcagtgaagtttacaaggcatttgatctaacagaacaaagatacgtagctgtgaaaattcaccagttaaataaaaactggagagatgagaaaaaggagaattaccacaagcatgcatgtagggaataccggattcacaaagaactggatcatcccagaatagttaagctgtatgattacttttcactggacactgactcgttttgtacagtactagaatactgtgagggaaatgatctggacttctacctgaaacagcacaaattaatgtcggag

SEQ ID NO: 105 - DNA, 873 bp, Bovine
ccaccgtacgccggtgctgggagtgcctgccttctcttgtcttgaaaacctcctctttggacccagcaccgccgtcctcacggtgatgttggactcagtgacacacagcaccttcctgcccaacacgtccttctgcgaccccctgatgtcgtggactgacctgttcagcaatgaagagtattaccctgcctttgagcatcagacagcttgcgactcctactggacatccgtccaccccgaatactggacgaagcgccacgtctgggaatggctccagttctgctgtgaccagtacaagctggacgccaactgcatctctttctgccatttcaacatcagtggcctgcagctgtgcggcatgacacaggaggagttcatggagcggccggcgtctgtggggagtatctgtactttatcctccagagcatccgctcacaaggttactccttttttaatgatcctgatgagaccaaggccaagcctccagagttctcatctatgggaatttgtgcgagatctgcttctatctcctgaggaaaactgcggcattctggaatgggaagctagggaacaaggtatttttcgggtggttaaatcagaagccctggcgaagatgtggggacaaaggaagaaaaatgacagaatgacgtacgaaaagctgagcagagctctgaggtactactataaaaccggaattttggaacgggttgaccgaagattagtgtacaaatttggaaaaaatgcacatgggtggcaggaagacaagctatgatctgctccatcatccagctcatgtaatggatttctgtcttttcaaacaatagattgcaatagacattggaaagtcctttaaaaaaaaaaaaaaaaaaaaaa

SEQ ID NO: 106 - DNA, 364 bp, Bovine
cagatactaaaggattttgcagtgccaatttgcttgaggacttgcctctgcaagagcctcagtcacctcacaagctcaatgcaggctttgacctggctaagggaggtgcaggtaaagtaaacctgcccaaagagctagctgcagatgctgtgaacatattacctgcctctctggacctctcccctctgttgggcttctggcagctgcctcctgctacccagaatgcctttgggagtagtggtcttgcctgggggctggggaatctctgccgcataggctgggctgtctggggcagcaagccccaagacccctcactagccatgagcactatgagcctgggccagcttcccctgcacccatcccc

SEQ ID NO: 107 - DNA, 1032 bp, Bovine
ggccgcggtggaagcgggtgcgcgggtcgcctctctgagttatccagttccatccttgtcgctgcggcgacacccgcattctccgtcgccatgactgaacagatgacccttcgtggcaccctcaagggccacaacggctgggtgacccagatcgctaccactccccagttcccggacatgatattgtccgcctctcgagataagaccatcattatgtggaagctgaccagagatgagaccaactatggtatcccacagcgtgctcttcggggtcactcccactttgttagtgatgtggtcatttcctcagatggccaatttgccctctcaggctcctgggatggaacccttcgcctttgggatctcacaacgggcaccaccactcgccgatttgtaggccataccaaagatgtgctgagtgtggccttctcttctgacaaccggcaaattgtctctggctcccgagacaaaaccatcaaactatggaatactctgggtgtatgcaagtatactgtccaggatgaaagccattcagagtgggtgtcttgtgtccgcttctcgcccaacagcagcaatcccattattgtttcctgtggctgggacaagctggtcaaggtatggaacttggcaaattgtaagctgaagaccaatcacatcggccacacaggctacctgaacactgtgaccgtctctccagatggatccctctgtgcttctggaggcaaggatgggcaggctatgttgtgggacctcaatgaaggcaagcacctttacacactagatggtggggacatcatcaacgccctgtgcttcagtcctaaccgctactggctctgtgctgccacgggtcctagcatcaagatctgggacttggagggcaagatcattgtagatgaactgaagcaagaagttatcagtaccagcagtaaagcagagcctccccaatgtacctctctggcctggtctgctgatggccagacactgtttgctggctacacggacaacctggtgcgagtgtggcaggtaccatcg

SEQ ID NO: 108 - DNA, 350 bp, Bovine
ttactacacaaccccgatctacaggttcaggatgaaatgccacctctgtgtcaactacatcgagatgcagacggaccccgccaactgtgactacgtgattgtgagcggcgcccagcgcaaggaggagcgctgggacatggaggacaacgaacaggtgctgaccacagagcatgagaagaagcagaagctggagatggacgccatgttccgcctggagcatggcgaggctgaccggagcacgctcaagaaggccctccccaccctgagccacatccaggaggcccagagcgcctggaaggacgacttcgccctcaacagcatgcttcggaaaaggttccgggaaaagaaaa

SEQ ID NO: 109 - DNA, 576 bp, Bovine
aatagagcgcaagctgcaacctgcgtttggctgtaacccaggagtaacgtcagaaacaggtgagaatgaccaccttaactcaccgggcccggtcgtactgaagtggggaagaattctgaaaagaaggtagaaagtgaggaaaacgtgaatcaagaccgaaatcaagacaatgaggacattggagactctaaggatatccgcctcacccttatggaagaagtattgctcctgggactaaaagataaagagggttacacatctttctggaatgactgcatatcatctggcctgcgaggcggcatcctgatagagctggccatgcgaggtcgaatctatctagaacccccaaccatgcgtaagaagcgactactagacagaaaggtactgctaaagtcagacagcccaacaggtgatgttttactggatgaaactctcaaacacatcaaggcaattgaacccacagaaactgtccaaacatggatagagctactcaccggtgagacctggaaccctttcaaattacagtaccagctgagaaatgtaagaaagagaattgcaaaacctagtagagaaggt

SEQ ID NO: 110 - DNA, 533 bp, Bovine
gggggaacggtctctgtttttctgcttcctccggcctcctcctcgaccgtggccgccgaccctcggaagcagtccgaacatgtccaacatggagaaacacctgttcaacttaaagttcgcggccaaagaactgggcaggagtgctaaaaaatgcgacaaggaggaaaaggccgaaaaggccaagattaaaaaggccattcagaagggcaatatggaagttgcgaggattcacgccgagaacgcgattcgccagaagaaccaggcagtgaatttcttgaggatgagcgcgcgggtggacgcggtggccgccagggtccagacggccgtgacgatgggcaaggtgaccaagtcgatggccggagtggttaagtcgatggacgcgacgttgaagaccatgaacctcgagaagatctccgccctgatggacaagttcgagcaccagttcgagacgctggacgttcagacgcagcagatggaggacacgatgagcagcacgacgacgctgaccactccccagggccaggtggacatgc

SEQ ID NO: 111 - DNA, 150 bp, Bovine
cgccttgtcgcttagattgttatggaggtctcatcgaatgttacttagcctccaacagtattcgtgaagcaatggtaatggctaacaatgtttacaaaactctaggagcaaatgcacagacccttacccttttagccaccgtttgtcttg

SEQ ID NO: 112 - DNA, 405 bp, Bovine
ctctggtggaggtccgtagcggtcctgacgtgcaaatcggtcgtccgacctgggtataggggcgaaagactaatcgaaccatctagtagctggttccctccgaagtttcccatggtgaaggtgacgttcaactcggctctggcccagaaggaggccaagaaggacgagtccaagagcggcgaggaggcgctcatcattcctccggacgccgttgccgtggactgcaaggacccagatgaagtggttccggttggccaaagaagagcttggtgttggtgcatgtgctttggactggcgtttatgctcgcaggtgtcattctggggggagcatacctgtacaaatactttgcatttcaaccagatgacgtctactactgtggaataaagtacatcaaagatgatgtc

SEQ ID NO: 113 - DNA, 1193 bp, Bovine
gagcagaacaagctgctggagaccaagtgggcgctgctgcaggagcagaagtctgccaagagcaaccgcctcccgggcatctttgaggcccagattgctggcctgcggaagcaactagaggccctgcagctggatgggggtcgcctggaggtggagcttcggaacatgcaggatgtcgtggaagacttcaagaataagtatgaagatgaaattaaccatcgcacagctgctgagaatgagtttgtggtgttgaagaaggatgtggatgttgcctacatgaacaaggtggagttggaggccaaggtggataccctgaatgatgagatcaacttcctcaggaccctctatgagcaggagctgaaagagctgcagtctgaggtctcagacacatccgtggtcctgtccatggacaacaaccgctccctggacttggacagcatcattgctgaagtcaaggcccagtatgaggagatcgccaaccgcagccgggcggaggccgaggcctgttaccagaccaagtttgagaccctccaggcccaggctgggaagcacggggacgacctccggaatacccggaatgagattgcggacatgaaccgggctgtccagaggctgcaggccgagatcgacagcgtcaagaaccagcgctccaagttggaagccgccattgccgatgctgaacagcgtggggaactggctgtcaaggatgcacgggccaagcaggaggatctggaggccgccctgcagaaggccaagcaggacatgacccggcagctgcgggagtaccaggagctcatgaacgtcaagctggccctggacatcgagatcgccacctaccgcaagctgctggagggcgaggagagccggttgaccggagatggagtgggagccgtgaacatctctgtggtcagttccacgggtggctctggtagcctgctgacctttggggggaccatgggcaacaacgccctgagattctccagcggtggagggcctgggaccctcaaggcctactccatgaggaccacatccgccacaagcaggagcccccgcaaatgagccctgggtgtggggagatacataccccctcctcccatagtcacaaggagacccccaaccctggtcccaccctcatcccaagaactgcaagacagtttgaaaaaagatatcagaatagcttccaataaagcagc

SEQ ID NO: 114 - DNA, 298 bp, Bovine
acgagttctaggtgagcgccagaggaaggaagaagagatgaagcagctatttgtgcagcgcgtgaaggagaaagaagccatcctgaaagaagctgagagagagctccaggccaagtttgaacaccttaaaagagtccaccaagaggagaagctgaggctggaagagaagagaagacttctggaagaagaaatcatggctttctccaagaagaaagctacttctgagatataccagaaccagacctttatgaccccaggcagcaacctgaggaaggacaaggaccgcaagaactccaat

SEQ ID NO: 115 - DNA, 446 bp, Bovine
attttgtttctccagaacatgttaagcactgcttctggcttactcaggaattccgctatctgtcacagactcatacaaaccacgaagataaattacaggtgaagaatgttatctaccatgcagtaaaagatgcggttgctatgctgaaagccagtgaatccagttttggcaaaccctgagggtcccagaggcaccttacccctgcacattggaagtgaattactggcagctgttcaaactcttcaggcaggattcctgtggactttgagactcatgttacctcatcttcttttttaaactgtacccacctggtaagggggtactctgtctaatgtatatttctagtgtttacagacactaaatgtgtatatgtagtaactatttacagaacatgcatcctttaaaactgtgacttctcacctagtgcagagcttctacccacctgt

SEQ ID NO: 116 - DNA, 332 bp, Bovine
aaacatttggatgtggatctggaccgccagtctttaagcagcattgataaaaatgcctctgagaggggtcagagccaactctctaacccgaccgatgacggctggaaagccagaccatatgcaaatcagaaactgtttgccagcctcctcatcaagtgtgtggtccagttggaactgatacagaccatcgacaacatcgtgttctaccccgcgacaagcaagagggaggacgcagagcacatggctgccatgccccagccggtacccacagcataataggtctccttggccgctggatctggaatctgaatgttgcttctgcaaagaccttt

SEQ ID NO: 117 - DNA, 575 bp, Bovine
agagagtaccacatcaccgtcgacgagcccagattgaagcagccaccctctggcttcgacagtgtcattgctcgtggccacacagagcctgatccaacccgggacaccgagctggagctagatggccagcgagtagtggtgccccagggccagcccgtgctctgcccagatttcagaagctgcaacttttcccagagcgaatatctcatctaccaggagagccagcgctgcctgcgctacctgctagagattcacctctgagctgtccgcccaaccctcccgccccctgggcaggagctcaaccatcatcctcaatcccatatcaacttcctgccctcacatctccctcctgtgttccagggattccctctccctctgatccttgttggttctggcatggctgtggcctcagtctcacctcctaaggtgatgggtgtgatggactgcaacacgaatacagcagcctattcaaggctgtgtagttaggggggcacaaactgagagtgcgtgggtcccacatccacccaggccatccgctgtggcctaactgtccatccaagagaggtgagatgatg

SEQ ID NO: 118 - DNA, 417 bp, Bovine
tgctaagtaagatttctcatgcaaaacctgcaattgcagattatgcatttacaacaataaagcctgaacttggaaaaattatgtatagtgatttcaaacagatatcagtagctgatcttcctggtttaatagaaggagcacatatgaacaaaggaatgggccacaaattcctcaagcatatagaaagaactaaacaactgctttttgttgttgatatttctggatttcagctttcttcccaaactcattacagaactgcttttgaaaccataatactgctttcaaaagagttagagttgtacaaagaggaacttcacacaaaacctgcactccttgcagtaaataaaatggacttgccagatgcacagggtaaattccatgtactgatgaaccaacttcagaattctaaagaatttt

SEQ ID NO: 119 - DNA, 377 bp, Bovine
gaaaccttgggatgatgagaccgatatggcaaaactagaggagtgcgtcagaagcattcaagcagatggcttggtctggggctcttctaaactagttccggttgggtatggcattaaaaaactacaaatacagtgtgtagttgaagacgacaaagttgggacagacatgctggaggagcagatcactgctttcgatgagtatgtacagtctatggatggaagacttggcgacaagtgctggttttttggctttctttgactcatactgggcaaggcgttcttctcttagcctcttggcttcttcactttcctcctcatcatcagatccaaagagatcaatgtcgcatcatctttactatctgtagctccactttctg

SEQ ID NO: 120 - DNA, 377 bp, Bovine; identical to SEQ ID NO: 100

SEQ ID NO: 121 - DNA, 363 bp, Bovine
caaaaatgtagtaagcagcattcagaaatacgtgaaaatttaataacagctttgtctacatggcagatgtttatagtggatatcaaaagaaataatacagcatttgatataattgcagataattgtgatctacattttaaaatatcaagagatcgcctcagtgcttcttcccttaccatggagagttttgcttttctttgggctggaggaagggcatcttacggtgtgtcaaaaggcaaagtctgttttgagatgaaggttacagagaagatcccggtgaggcatttatatacgaaagatattgatatcatgaagttcggattgggtggtcactaaccacaagtggaatgttgcttggtaaag

SEQ ID NO: 122 - DNA, 501 bp, Bovine
cttattttgtgacggattatgatccaaccatcgaggattcctacacaaagcagtgtgtgatagatgaccgggcagcccggctcgacattctggatacagcgggacaagaggaatttggagctatgagagaacagtatatgagaactggcgagggtttccttttggtctcttcagtcacagacagaggcagttttgaagaaatctataagtttcaaagacagattctcagagtaaaggatcgtgatgagtttccaatgattttaattggtaataaagcagatctggatcatcagagacaggtaacacaggaagaaggacagcagttagcacggcaacttaaggtaacatacatggaggcgtcagcaaagattagaatgaatgtagatcaagctttccatgaacttgtccgggttataaggaaatttcaagagcaggaatgtcctccttcaccagaaccaacacggaaaggaaaaagacaagaacggctgcattgtgtcattt

SEQ ID NO: 123 - DNA, 414 bp, Bovine
ggaagcgtgaggcccactcgaattccgtttggaggctcctttgagctcgcggagtagacatgagcaaagcacaccctcccgagttgaagaaatttatggacaagaagttatcattgaaattaaatggtggcagacatgtccaaggaatattgcggggatttgatccctttatgaatcttgtgatagatgaatgtgtggagatggcaactagtgggcaacagaacaatattggaatggtggtaatacgaggaaatagtatcatcatgttagaagccttggaacgagtatgaacaatggctgtgttcaccagagaaatcaacgcttccatgtgtgccctctccacattttactaccagaaaaattgggttgtgtacattttcttacttttttgttaaataaacttttgtaatagcc

SEQ ID NO: 124 - DNA, 1369 bp, Bovine
gttaatgcccgtctcctcagaataagagacttgatggcagcggcctttagcagcctgctttggatcaggcactggagtagacgtccctagcagaggggtcttctgggcagccggagacctaccgaaggcggcagtgatggcggcccggcgggatggatggcttggcccggcgtttgggctgcgactgctgctggcgaccgtgcttcaaacggtctctgcattgggagcagaattttcctcagagtcttgcagagagttaggtttctccagcaacttgctctgcagctcctgtgatttgcttggacagttcaaccttctgcagctggatcctgactgtagagggtgctgtcaggaagaagcacaatttgaaactaaaaagctgtatgcaggagctattcttgaagtctgtggatgaaaattggggaggttccctcaagtccaagctttcgtcaggagtgataaacccaagctattcaaaggactacaaatcaagtatgttcgcggttcagaccctgtgctaaagcttttggacgacagtgggaacattgctgaagaactaagcatcctcaaatggaacacagacagtgtagaagaattcctgagtgaaaagttggaacgcatataaatcttaaattttgtcctatctttctgttaccttgttcaatgaaacactatagcacctagaaaataatttagttttgctttcttccattgatcagtcttttactgtgaggcattaaacatctaattaaaagttcaagcagcagcgcagcccatgataggtaagaagttaacaaaacccttttttataagtttccatccatcatttgttgataccactagtaacagaatgccttctaatagacttgtggttaattatgcaaatgatagtttgtgataattggtcagttttatgaacaacagatttttaaattaaggaggttaataagggagatgattattgtgcctcgtgtgctgtgtgctctttgaaagtaacaacaacaaacttgaaagcaagtaagacataccgagcctcaagagattgcctgttccccagatcctctcatatttttgtacacccagccttccttttaatagaaatgtatagtttataatgaatgcactgcataaagactttatggctgcattattgtaaaacagattcaagatctacagtaagagtgaaatattcacacaaagatttgcattaatgaagactacacagaaaaccttcctgaggatttgtgtggacctgatacttagcaaatttttgtgctttacattcttatggaaaagtcaatttaaaaatgatcatttgtaagaccaaaatataaataaaaagtttcaaaaatcaaaaaaaaaaaaaaaa

SEQ ID NO: 125 - DNA, 327 bp, Bovine
caagactgacatgttccagaccgttgacctctttgaaggcaaagacctggctgcggtgcaaaggaccttgatggccctgggcagcttggcagtgaccaagaatgatggacactaccgtggagatcccaactggtttatgaagaaagcccaggagcataagagggaattcacagagagccagctgcaggagggcaagcatgtcatcggcctacagatgggcagcaacagaggggcctcgcaggctggcatgacgggctatgggcggccccggcagatcatcagttaagagggtgagggccggcgcccagccccacctttgccagctcg

SEQ ID NO: 126 - DNA, 740 bp, Bovine
gagcatatgcttgtctcaaagattaagccatgcatgtctaagtacgcacggccggtacagtgaaaccgcgaatggctcattaaatcagttatggttcctttggtcgctcgctcctctcctacttggataactgtggtaattctagagctaatacatgccgacgggcgctgacccccttcgcgggggggatgcgtgcatttatcagatcaaaaccaacccggtcagcctcctcccggccccggccggggggcgggcgccggcggctttggtgactctagataacctcgggccgatcgcacgccccccgtggcggcgacgacccattcgaacgtccgccctatcaactttcgatggtagtcgctgtgcctaccatggtcacccggggccaggacgtgggacgttaccaggtgtcctggagcctagatcacaagagcgcccatgcgggcacctatgaggtccggttctttgatgaggagtcctatagcctcttgaggaaggctcagagaaacaatgaggacgtttctgtcatcccacctctattcacagtcagcgtagaccatcggggtacctggaacgggccctgggtctccactgaagtcctggccgcagccatcggactagtgatctactaccttgccttcagcgccaagagccacatccaggcctgagggtggtgcccgcccccacccttgcttctttgaataaagagctattggctatcctgaaaaaaaaaaaaaaaa

SEQ ID NO: 127 - DNA, 315 bp, Bovine
agctttcgtgatatttactttgacacccttaatgaagatcttttccagaaaatacttgtacccattcagcaagtgttgaaagaaggccacctggaaaagactgagattgatgaggtggtcttggttgggggctcaactcgtattcctcgaatccgccaagtcatccaagagttctttggaaaggaccccaacacgtctgtagaccctgacctggccgtggtgacaggagtggctatccaggcagggattgatggaggctcttggcctctccaagtcagtgctttagaaattcccaataagcatttacagaagacc

SEQ ID NO: 128 - DNA, 390 bp, Bovine
tggcacctgggacagcttcctggagaagttcatggccggggaagtgtgctacgggtcctggtaccagcacgtgcatgagtggtgggagctgagtcacacccaccctgttctctacctcttctacgaggacataatggaggaccccaaaagggagattcagaagatcctggagtttatagggcgctccctgcctgaggagactgtggatcacattgtccagcgaccatacccactgcagtcatggaccacaagcatctcttccttcatgaggaaaggcatcactggtgattggaaatccaccttcactgtggcccagaatgagctctttgaagcccactatgctaagaagatgcgggctgcaagcttccgctttcgctggaagctgtgagt

SEQ ID NO: 129 - DNA, 228 bp, Bovine
caaaaaaaggcatctgcttctgcaggaagaataacagttccacggttaagtgttggttcagttactagcagaccaagtactcccacgcttggcacaccaaccccaccagccatgtccgtttcaactaaagtagggactccagtgtccctcacagggcaaaggttcacagtacagatgcccacttcacagtccccagctgtaaaagcttcaattcctgcaacatcagca

SEQ ID NO: 130 - DNA, 580 bp, Bovine
gcagccacgctgaaccggctcagggcttttcgccacgccgtgtcctctgatcttcctctgctgggtcagttatggcggccgtgaagaccctgaaccccaaggccgaggtggcccgagcccaggcggcgttggcggtcaacatcagcgcggcccgggggctgcaggacgtgctgaggaccaacttggggcctaagggcaccatgaagatgcttgtttctggtgctggagacatcaaactcactaaagatggaaatgtgctgcttcatgaaatgcaaattcagcacccaacagcctccttaatagccaaagtagcaacagcccaggatgacataactggtgatggcaccacttccaatgtcctcatcatcggagagctgctgaagcaggcggatctctacatttctgagggtcttcatcccagaataattacagaaggatttgaagctgcaaaggaaaaggcacttcagttcttggaacaagtcaaagtaagcaaagagatggacagggaaacacttatagacgtggccagaacatctctacgtactaaagttcatgctgaacttgcagatgtcttaacaga

SEQ ID NO: 131 - DNA, 679 bp, Bovine
ggttgtctccaatttctcctcgccccctctccccgtcccagccaagatgtctgacatggaggatgatttcatgtgcgatgatgaggaggactacgacctggaatactctgaagatagtaactctgagccaaatgtggatttggaaaatcagtactataattccaaagcattaaaagaagatgacccaaaagcagcattaagcagtttccaaaaggttttggaacttgaaggtgaaaaaggagaatggggatttaaagcactgaaacaaatgattaagattaacttcaagttgacaaactttccagaaatgatgaacagatataaacaactattgacctatattcggagtgctgtcacaagaaattattctgaaaaatccattaattctattcttgattatatctctacttctaagcagaattctgattttttatgtcagatggatttactgcaggaattctatgaaacaacactggaagctttgaaagatgctaagaatgacacactgtggtttaagacaaacacaaagctggggaaattatatttagaacgagaggaatatggaaaacttcaaaaaattttacgccagttacatcagtcctgccagactgatgatggagaagatgacctgaaaaaggcacacagttattagaaatatatgctttggaaattcaaatgt

SEQ ID NO: 132 - protein, 226 aa, Bovine
CTCLDGSVGCVPLCSVDVRLPSPDCPFPRRVKLPGKCCEEWVCDEPKEHTVVGPALAAYRPEDTFGPDPTMIRANCLVQTTEWSACSKTCGMGISTRVTNDNAFCRLEKQSRLCMVRPCEADLEENIKKGKKCIRTPKISKPIKFELSGCTSMKTYRAKFCGVCTDGRCCTPHRTTTLPVEFKCPDGEVMKKSMMFIKTCACHYNCPGDNDIFESLYYRKMYGDMA

SEQ ID NO: 133 - protein, 103 aa, Bovine
MSYGRPPPDVEGMTSLKVDNLTYRTSPDTLRRVFEKYGRVGDVYIPRDRYTKESRGFAFVRFHDKRDAEDAMDAMDGAVLDGRELRVQMARYGRPRIRTIAAG

SEQ ID NO: 134 - protein, 84 aa, Bovine
MPYLLISTQIRMEVGPTVVGDEHSDPELMQHLGASKRSVLGNNFSEYYVNDPPRIVLDKLERRGFRVLSMTGVGQTLVWCLHKE

SEQ ID NO: 135 - protein, 189 aa, Bovine
MLDSVTHSTFLPNTSFCDPLMSWTDLFSNEEYYPAFEHQTACDSYWTSVHPEYWTKRHVWEWLQFCCDQYKLDANCISFCHFNISGLQLCGMTQEEFMEAAGVCGEYLYFILQSIRSQGYSFFNDPDETKATLKDYADSSCLKTSGIKSQDCHSHSRTSLQSSHLWEFVRDLLLSPEENCGILEWEDKE

SEQ ID NO: 136 - protein, 85 aa, Bovine
ADSSCLKTSGIKSQDCHSHSRTSLQSSHLWEFVRDLLLSPEENCGILEWEDREQGIFRVVKSEALAKMWGQRKKNDRMTYEKLSR

SEQ ID NO: 137 - protein, 101 aa, Bovine
LATLAQRVKEVLPHVPLGVIQRDLARTGCVDLTITNLLEGAVAFMPEDITEGTQSLATASTPKFPSSGPATPQPTALTFAKSSWARQESLQERKQALYECA

SEQ ID NO: 138 - protein, 73 aa, Bovine
SFPQRMSSFQLNLNPLKEPLGFIKVLEWIASIFAFATCGGFKGKTEIQVSCTTGPENKTITAAFGYPFRLNEA

SEQ ID NO: 139 - protein, 124 aa, Bovine
MADDLKRFLYKKLPSVEGLHAIVVSDRDGVPVIKVANDNAPEHALRPGFLSTFALATDQGSKLGLSKNKSIICYYNTYQVVQFNRLPLVVSFIASSNANTGLIVSLEKELAPLFEELRQVVEVS

SEQ ID NO: 140 - protein, 88 aa, Bovine
QPAKLAEAFKYFVQGMGYMPSASMTRLMRSRTASGSSVTSLEGARSRSHTSEGTRSRSHTSEGTRLDIIPNSGGPGSSAGPNSTEVSC

SEQ ID NO: 141 - protein, 86 aa, Bovine
MVYISNGQVLDSRSQSPWRLSFITDFFWGIAEFVVLFFRTLLQQDVKKRRGYGSSSDSRYDDGRGPPGNPPRRRMGRINHLQGPNP

SEQ ID NO: 142 - protein, 69 aa, Bovine
MFGYAVRRALRKSKTLRYGVPMLLLIVGGSFGLREFSQIRYDAVKIKIDPELEKKLKMNKVSLESEYEK

SEQ ID NO: 143 - protein, 257 aa, Bovine
MTQIMFETFNTPAMYVAIQAVLSLYASGRTTGIVMDSGDGVTHTVPIYEGYALPHAILRLDLAGRDLTDYLMKILTERGYSFTTTAEREIVRDIKEKPCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 144 - protein, 212 aa, Bovine
KTVAVPCIIQDSSSCCVPNCEPSLSVQPPALEDLLLGSNASLTCTLSGLKSAEGASFTWNPTGGKTAVQGSPKRDSCGCYSVSSVLPGCADPWNSGQTFSCSVTHPESKSSLTATIKKDLGNTFRPQVHLLPPPSEELALNELVTLTCLVRGFNPKEVLVRWLQGNQELPREKYLTWAPCPSWPERTTFAVTNVLRVDAEVWKQGDTFSAWW

SEQ ID NO: 145 - protein, 148 aa, Bovine
MVMVLSPLFLVFILGLGLTPVAPAQDDYRYIHFLTQHYDAKPKGRNDEYCFNMMKNRRLTRPCKDRNTFIHGNKNDIKAICEDRNGQPYRGDLRISKSEFQITICKHKGGSSRPPCRYGATEDSRVIVVGCENGLPVHFDESFITPRH

SEQ ID NO: 146 - protein, 140 aa, Bovine
RFMLLFSRQGKLRLQKWYLATSDKERKKMVRELMQVVLARKPKMCSFLEWRDLKVVYKRYASLYFCCAIEGQDNELITLELIHRYVELLDKYFGSVCELDIIFNFEKAYFILDEFLMGGDVQDTSKKSVLKAIEQADLLQ

SEQ ID NO: 147 - protein, 103 aa, Bovine
VQVICMKGKAKYKASENAIVWKIKRMAGMKESQISAEIELLPTNDKKKWARPPISMNFEVPFAPSGLKVRYLKVFEPKLNYSDHDVIKWVRYIGRSGIYETRC

SEQ ID NO: 148 - protein, 147 aa, Bovine
PAAAMILLEVNNRIIEETLALKFENAAAGNKPEAVEVTFADFDGVLFSHREPPLELKDTDAAVGDNIGYITFVLFPRHTNASARDNTINLIHTFRDYLHYHIKCSKAYIHTRMRAKTSDFLKVLNRARPDAEKKEMKTITGKTFSSR

SEQ ID NO: 149 - protein, 77 aa, Bovine
FMTHPEFRIEDSEPHIPLIDDTDAEDDAPTKRNSSPPPSPNKNNNAVDSGIYLTIEMNKSATSSSPGSPLHSLETSL

SEQ ID NO: 150 - protein, 148 aa, Bovine
  VARIANT (1)...(148): Xaa = Any Amino Acid
MNENLFTSFITPVILGLPLVTLIVLFPSLLFPTSNRLVSNRFVTLQQXILQLVSKQIMSIHNSKGQTXTLILISLILFIGSTNLLGLLPHSFTPTTQLSINLGIAIPLXAGAVITGFRNKTKASLAHFLPQGTPTPLIPILVIIETIS

SEQ ID NO: 151 - protein, 71 aa, Bovine
MVPPVQVSPLIKLGRYSALFLGMAYGAKRYNYLKPRAEEERRLAAEEKKKRDEQKRIERELAEAQEDTILK

SEQ ID NO: 152 - protein, 173 aa, Bovine
RGAAEEGPGDGGEAMWQLLLPLALGLGTMGLGRAELTTAQHRGLQVALEEFHKHPPVLWAFQVTSVDNAADTLFPAGQFVRLEFKLQQTSCRKKDWRKEDCKVKPNGRKRKCLACIKLDSKDQVLGRMVHCPIQTQELDDAQDAQCSRVERAGEDPHSYYLPGQFAFIKALSP

SEQ ID NO: 153 - protein, 124 aa, Bovine
CRPSHPVCSTTVSCVSAEGSAQRGPGPWPPCPAACCGEWWRATALALLSSLDALQVCVCTCGRAWAWPCFLAGKHVGPGVAGPLRCTSGAGGDPSPPRETELSSNMMVLNDILTSFDENCHFSM

SEQ ID NO: 154 - protein, 100 aa, Bovine
EEWSCCIRNLLLGQEKDVEVSIPASFFPRLTPWMVAVAVILVVLGLLTIGSIFFTWRLYKERSRQRRNEFSSKEKLLEELKWKRATLHAVDVTLDPDTAH

SEQ ID NO: 155 - protein, 110 aa, Bovine
GRPALHLVALNTPFSGDIRADFQCFQQARAAGLLSTYRAFLSSHLQDLSTVVRKAERYSLPIVNLKGQVLFNNWDSIFSGHGGQFNTHIPIYSFDGPDVMTDLSGPEGIL

SEQ ID NO: 156 - protein, 217 aa, Bovine
MSSKVSRDTLYEAVREVLHGNQRKRRKFLETVELQISLKNYDPQKDKRFSGTVRLKSTPRPKFSVCVLGDQQHCDEAKAVDIPHMDIEALKKLNKNKKLVKKLAKKYDAFLASESLIKQIPRILGPGLNKAGKFPSLLTHNENMVAKVDEVKSTIKFQMKKVLCLAVAVGHVKMTDDELVYNIHLAVNFLVSLLKKNWQNVRALYIKNTMGKPQRLY

SEQ ID NO: 157 - protein, 142 aa, Bovine
MASKRALVILAKGAEEMETVIPVDVMRRAGIKVTVAGLAGKDPVQCSRDVVICPDASLEDAKKEGPYDVVVLPGGNLGAQNLSESAAVKEILKEQEKRKGLIAAICAGPTALLAHEIGFGSKVTTHPLAKDKMMNGSHYSYS

SEQ ID NO: 158 - protein, 65 aa, Bovine
KPQFISRGTFNPEKGKQKLKNVKNSPQKTKETPEGIVVSSRRKTVDPDCSSAQQLALFGNNEFMV

SEQ ID NO: 159 - protein, 88 aa, Bovine
MPAATVDHSQRICEVWACNLDEEMKKIRQVIRKYNYVAMDTEFPGVVARPIGEFRSNADYQYQLLRCNVDLLKIIQLGLTFMNEQENT

SEQ ID NO: 160 - protein, 176 aa, Bovine
MNWLVWALLLCSSAMAHVHRDPTLDHHWDLWKKTYGKQYKEKNEEVARRLIWEKNLKTVTLHNLEHSMGMHSYELGMNHLGDMTSEEVISLMSSLRVPSQWPRNVTYKSDPNQKLPDSMDWREKGCVTEVKYQGACGSCWAFSAVGALEAQVKLKTGKLVSLSAQNLVDCSTAKYG

SEQ ID NO: 161 - protein, 104 aa, Bovine
GHLYTVPIREQGNIYKPNNKAMAEEMNEKQVYDAHTKEIDLVNRDPKHLNDDVVKIDFEDVIAEPEGTHSFDGIWKASFTTFTVTKYWFYRLLSASLASQWHSS

SEQ ID NO: 162 - protein, 244 aa, Bovine
MALFTVVLFLAAVWLPFFPAKGQDRRFADLSNTLKNVQTEIVNKHNDLRRGVSPPPSNMLKMQWNTTAAANAQNWANKCLFKHSKKEDRRVGTRNCGENLFMSSYPSTWSNAIQSWYDEVHDFVFEVGPKSPQAVIGHFTQIVWYSSFLIGCGVAYCPKQSLKYLYVCQYCPAGNIVGRQHVPYQKGTPCGSCPNHCDNGLCTNSCEYEDTYSNCASLKETWTCASDFVKTNCKAACNCQGKIY

SEQ ID NO: 163 - protein, 226 aa, Bovine; identical to SEQ ID NO: 132

SEQ ID NO: 164 - protein, 164 aa, Bovine
MVNPTVFFDIAVDGEPLGRVSFELFADKVPKTAENFRALSTGEKGFGYKGSCFHRIIPGFMCQGGDFTRHNGTGGKSIYGEKFDDENFILKHTGPGILSMANAGPNTNGSQFFICTAKTEWLDGKHVVFGKVKEGMNIVEAMERFGSRNGKTSKKITIADCGQI

SEQ ID NO: 165 - protein, 94 aa, Bovine
HELERTGHYLTVKDNQVVQLHPSTVLDHKPEWVLYNEFVLTTKNYIRTCTDIKPEWLVKIAPQYYDMSNFPQCEAKRQLDRIIAKLQSKEYSQY

SEQ ID NO: 166 - protein, 103 aa, Bovine
MAAFSEMGVMPEIAQAVEEMDWLLPTDIQAESIPLILGGGDVLMAAETGSGKTGAFSIPVIQIVYETLKDQQEGKKGKATIKTGASVLNKWENDECAQKKIIA

SEQ ID NO: 167 - protein, 136 aa, Bovine
MAGKKVLIVYAHQEPRSLNGSLKDVAVAELSQQGCSVIVSDLYAMNFEPRATGKDITGTLSNPGFFNYGVEAHKAYKKQSLSSDIIEEQKKLQEADLVIFQFPLYWFSVPAVLKGWMDRVLCQGFAFDFPGSYDDG

SEQ ID NO: 168 - protein, 105 aa, Bovine
APLHSVLSNVEVTLNVLADSVLMEQPPLRRRKLEHLITELVHQRDVTRSLIKSRVDNAKSFEWLSQMRFYFDPKQTDVLQQLSIQMANAKFNYGFEYLGVQDKAG

SEQ ID NO: 169 - protein, 303 aa, Bovine
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMRHEALSEAL

SEQ ID NO: 170 - protein, 93 aa, Bovine
WFLTCINQPQFRAVLGEVKLCEKMAQFDAKKFAESQPKKDTPRKEKGSREEKLKPQAERKEGKEEKKAAAPAPEEELDECEQALAAEPKAKDP

SEQ ID NO: 171 - protein, 55 aa, Bovine
NKYDDDGEGITLFRPSHLTNKFEDKTVAYTEQKMTSGKIKRFIQENIFGICPHMT

SEQ ID NO: 172 - protein, 132 aa, Bovine
MCDAFVGTWKLVSSENFDDYMKEVGVGFATRKVAGMAKPTLIISLNGGVVTIKSESTFKNTEISFKLGQEFDEITPDDRKVKSIVNLDEGALVQVQNWDGKSTTIKRKLVDDKMVLECVMNGVTATTVYERA

SEQ ID NO: 173 - protein, 138 aa, Bovine
MVDAFVGTWKLVDSKNFDDYMKSLGVGFATRQVGNMTKPTTIIEVNGDTVIIKTQSTFKNTEISFKLGVEFDETTADDRKVKSIVTLDGGKLVHVQKWNGQETSLVREMVDGNFILTLTHGTASCTRTYENSMTASLH

SEQ ID NO: 174 - protein, 181 aa, Bovine
MTTASPSQVRQNYHQDSEAAINRQINLELYASYVYLSMSYYFDRDDVALKNFAKYFLHQSHEEREHAERLMKLQNQRGGRIFLQDIKKPDRDDWENGLTAMECALCLERSVNQSLLELHKLATEKNDPHLCDFIETHYLNEQVEAIKELGDHITNLRKMGAPGSGMAEYLFDKHTLGHSES

SEQ ID NO: 175 - protein, 203 aa, Bovine
RTKLMLMSRNEEATKHLECTKQLAAAFHEEFVVREDLMGLAIGTHGSNIQQARKVPGVTAIELDEDTGTFRIYGESADAVKKARGFLEFVEDFIQVPRNLVGKVIGKNGKVIQEIVDKSGVVRVRIEGDNENKLPREDGMVPFVFVGTKEKPWEMCKCFSEYHIAYLKEVQQLRMEPPSRLMEQLRPDLVWAFRPFFPPRGAL

SEQ ID NO: 176 - protein, 110 aa, Bovine
MTLEELRGQDTVPESTARMQGAGKALHELLLSAQRQGCLTAGVYESAKVLNVDPDNVTFCVLAADEEDEGDIALQIHFTLIQAFCCENDIDIVRVGDVQRLAAIVGTGDE

SEQ ID NO: 177 - protein, 117 aa, Bovine
ELLAKHKSLPWKEVLRLEEVQAKLGISLEEMLLITEDALHPEPYSPEEICKCLGISLQELKTQILSPNTQDVLTFKLYQRAKHVYSEAARVLQFKKICEEAPDNVVQLLGELMNQSH

SEQ ID NO: 178 - protein, 197 aa, Bovine
MTEQMTLRGTLKGHNGWVTQIATTPQFPDMILSASRDKTIIMWKLTRDETNYGIPQRALRGHSHFVSDVVISSDGQFALSGSWDGTLRLWDLTTGTTTRRFVGHTKDVLSVAFSSDNRQIVSGSRDKTIKLWNTLGVCKYTVQDESHSEWVSCVRFSPNSSNPIIVSCGWDKLVKVWNLANCKAEDQSHRPHRLPEH

SEQ ID NO: 179 - protein, 266 aa, Bovine
ALSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDNVEVNTATTKPREEQFNSTYRVVSALRIQHQDWTGGKEFTCKVHNEGLPAPIVRTISRTKGQAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 180 - protein, 212 aa, Bovine
RVPTTPKTTIPPGKPTTQESEVEKTPCQCSKCPEPLGGLSVFIFPPKPKDTLTISGTPEVTCVVVDVGQDDPEVQFSWFVDDVEVHTARTKPREEQFNSTYRVVSALRIQHQDWLQGKEFKCKVNNKGLPAPIVRTISRTKGQAREPQVYVLAPPREELSKSTLSLTCLITGFYPEEIDVEWQRNGQPESEDKYHTTAPQLDADGFLLSVQE

SEQ ID NO: 181 - protein, 131 aa, Bovine
NTQHETVTYLPGHKLPPNVVAVPDVVQAAADADILIFVVPHQFIGKICDQLKGHLKADTIGVSLIKGVDEGPKGLKLISEVIGERLGIPMSVLMGANIANEVADETFCETTIGSKNQAHGQLLKELMQTPN

SEQ ID NO: 182 - protein, 104 aa, Bovine
DPWPEPRPPPPPGSSAQRCCSCSWWPPAGAQQVRPGARDPLGRTGTGGYPWGQPLTHSVLPAGAPVVNELRCHCLQTLQGIHLKNIQSVKVTPPGPHCGQTEVM

SEQ ID NO: 183 - protein, 79 aa, Bovine
HISLADLVAITELMHPVGAGCQVFKGRPKLAAWRQRVEAAVGEVLFQEAHEVILKAKDSQPADPTLKQKMLPKVLAMIQ

SEQ ID NO: 184 - protein, 115 aa, Bovine
GSGTTLTVLGQPKSAPSVTLFPPSKEELDTNKATLVCLISDFYPGSVTVVWKADGSTITRDVKTTRPSKQSNSKYAASSYLSLTDSDWKSKGSYSCEVTHDGSTVTKTVKPSECP

SEQ ID NO: 185 - protein, 160 aa, Bovine
SGVPDRFSGSKSGDTATLTISSLQAEDEADYFCGTGDYSINIVVFGSGTTLTVLGQPKSAPSVTLFPPSKEELDTNKATLVCLISDFYPGSVTVVWKADGSTITRDVKTTRPSKQSNSKYAASSYLSLTDSDWKSKGSYSCEVTHDGSTVTKTVKPSECP

SEQ ID NO: 186 - protein, 136 aa, Bovine
RATGDFDSKPSWADQVEEEGEDDKCVTSELLKGIPLATGDTSPEPELLPGAPLPPPKEVINGNIKTVTEYKIDEDGKKFKIVRTFRIETRKASKAVARRKNWKKFGNSEFDPPGPNVATTTVSDDVSMTFITSKED

SEQ ID NO: 187 - protein, 161 aa, Bovine
YTGVPDRFTGSGSETDFTLTISNVQAEDAGVYYCLQSTYTPHTFGQGTKVEIKGSDAEPSVFLFKPSDEQLKTGTVSVVCLVNDFYPKDINVKWKVDGVTQSSSNFQNSFTDQDSKKSTYSLSSILTLPSSEYQSHDAYTCEVSHKSLTTTLVKSFSKNEC

SEQ ID NO: 188 - protein, 185 aa, Bovine
GYVSWYQLTPGSAPRTLMYGDTGLASGVPDRFSDSRSGNTATLTINSLQAEDEADYFCASAEESSSKVLFGSGTTVTVLGQPKSPPSVTLFPPSTEELNGNKATLVCLISDFYPGSVTVVWKADGSTITRNVETTRASKQSNSKYAASSYLSLTSSDWKSKGSYSCEVTHEGSTVTKTVKPSECS

SEQ ID NO: 189 - protein, 115 aa, Bovine
LKEKLIAPVAEEETRIPNNKITVVGVGQVGMACAISILGKSLTDELALVDVLEDKLKGEMMDLQHGSLFLQTPKIVADKDYSVTANSKIVVVTAGVRQQEGESRLNLVQRNVNVF

SEQ ID NO: 190 - protein, 119 aa, Bovine
ALGSAGLLFCPRSRLVPCISYRGTSPEMESKALLLLALSVCLQSLTVSRGGLVAADRITGGKDFRDIESKFALRTPEDTAEDTCHLIPGVTESVANCHFNHSSKTFVGIHGWTVTGMYE

SEQ ID NO: 191 - protein, 102 aa, Bovine
MRLSVTALLGTLALCYYKANAIVCPTFAADLTEFFYFPDLLYRLSLAKYNAPPEAVAAKMEVKQCTDRFSVKNRLIITNILGKILLNCTVTDVKAVLNPSSA

SEQ ID NO: 192 - protein, 155 aa, Bovine
ITCSGTSSNVGDGDYVSWFQQIPGSGPRTVIFGATQRPSGVSERFSGSRSGNTATLTISSLQAEDEADYFCSSPDTTNNVAFGSGTTLSVLRQRLEIERQLQLRGHARREHRDEDSEALRVSLGPWTPTLGGPLAHTPSPTSPWTPEPLPRSPTP

SEQ ID NO: 193 - protein, 102 aa, Bovine
LVYDFANFGVLRLSEPAPLFDLAMLALDSPESGWTEEDGPKEGLAEYIVEFLKKKAEMLADYFSLEIDEEGNLVGLPLLIDNYVPPLEGLPIFILRLATEVN

SEQ ID NO: 194 - protein, 132 aa, Bovine
ISYQVGWLIPVFCYRIFDFVLSCLVAISSLTYLPRIKEYLDQLPDFPYKDDLLALDSSCLLFIVLVFFALFIIFKAYLINCVWNCYKYINNRNMPEIAVYPAFEAPPQYVLPTYEMAVKMPEKEPPPPYIPA

SEQ ID NO: 195 - protein, 233 aa, Bovine
APIGVFTIPPSFADIFLTKSAKLSCLVTNLASYDGLNISWSRQNGKALETHTYFGRHLNDTFSARGEASVCSEDWESGEEFTCTVAHSDLPFPEKNSVSKPKDVAMKPPSVYLLPPTREQLSLRESASVTCLVKGFAPADVFVQWLQRGEPVTKSKYVTSSPAPEPQDPSVYFVHSILTVAEEDWSKGETYTCVVGHEALPHMVTERTVDKSTGKPTLYNVSLVLSDTASTCY

SEQ ID NO: 196 - protein, 248 aa, Bovine
PGPGPGPGSNLTSAPGPSTTTRSLTACPEESPLLVGPMLIEFNIPVDLKLVEHQNPKVKLGGRYTPTDCISPHKVAIIIPFRNRQEHLKYWLYYLHPILQRQQLDYGIYVINQAGESMFNRAKLLNVGFKEALKDYDYNCFVFSDVDLIPMNDHNTYRCFSQPRHISVAMDKFGFSLPYVQYFGGVSALSKQQFLSINGFPNNYWGWGGEDDDIYNRLDFKGMSVSRPNAVIGKCRMIRTRETKKNEP

SEQ ID NO: 197 - protein, 272 aa, Bovine
MEDSMDMDMSPLRPQNYLFGCELKADRDYHFKVDNDENEHQLSLRTVSLGAGAKDELHVVEAEAMNYEGSPIKVTLATLKMSVQPTVSLGGFEITPPVVLRLKCGSGPVHISGQHLVAVEEDAESEEEEEEEVKLLSISGKRSAPGSGSKVPQKKVKLAADEDEDDDDDDDDDDDEDDDDDDFDEEVEEKAPVKKSVRDTPAKNAQKSNQNGKDSKPSTPRSKGQESFKKQEKTPKTPKGPSSVEDIKAKMQASIEKGGSLPKVEAKFINYV

SEQ ID NO: 198 - protein, 108 aa, Bovine
  VARIANT (1)...(108): Xaa = Any Amino Acid
AIQKKKKKAGGITCPDFKYYKATVIQIAWYWHKSRHVDQXIRAESPEISPHTYSQSVFDRTDKDLQWRNDGLFSKRCWESWACMCAQSLSLAAYKSIKLDTASHHTQK

SEQ ID NO: 199 - protein, 139 aa, Bovine
EKLKEAPEGTFLIRDSSHSDYLLTISVKTSAGPTNLRIEYQDGKFRLDSIICVKSKLKQFDSVVHLIDYYVQMCKDKRTGPEAPRNGTVHLYLTKPLYTSAPPLQHLCRLTINKCTSTVWGLPLPTRLKDYLEEYKFQV

SEQ ID NO: 200 - protein, 195 aa, Bovine
ETGVLKPGMVVTFAPVNVTTEVKSVKMHHEALSEALPGDNVGFNVKNVSVKDVRRGNVAGDSKNDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 201 - protein, 196 aa, Bovine
DLDALVQFLSIGTLLAYTFMAISVLVLRFQTASQSRSPSLAGSGPKAKEYSSFSDHLELVGAGHGPEPGRLRPALRPYLGFLDRGSPGAAVRGAVCGLVVSAIALGCVLMLGHSVLRLPLWGFLLLLLCSSVTFLLSLLVLGAHQQQRLKDTFQMPLVPLIPALSIVLNFCLMLKLSYLTWVRFTIWLLIGLLVYF

SEQ ID NO: 202 - protein, 124 aa, Bovine
FYVSQPGSSVVTSLSPGEAVKKHIGLLRIKGRKMNMQKIPLRTVRQFFMEDVVLADHPDIFNPDNPKVTQVIQNFCLEKVEEMLENAERERLGNSQQPEKPLIRLRVDYSGGFEPFSVLRFSQK

SEQ ID NO: 203 - protein, 114 aa, Bovine
PTMLQDPDVREFLEKEELPRAVGTQTLSGAGLLKMFNKATDAVSKMTIKMNESDIWFEEKLQEVECEEQRLRKLHAVVETLVNHRKELALNTAQFAKSLAMLGSSEDNTALSRA

SEQ ID NO: 204 - protein, 152 aa, Bovine
MIHNYMEHLERTKLHQISGSDQLESTAHSRIRKERPISLGIFPLPSGDGLLTPDTQKGGETPGSEQWKFQELSQPRSHTSLKDELSDVSQGGSKATTPASTAASDVAATPSDTPLHEENGGVVEVADTPDKSEISKHISIPLTETNKTSGAS

SEQ ID NO: 205 - protein, 219 aa, Bovine
ALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELSEDEEE

SEQ ID NO: 206 - protein, 187 aa, Bovine
GNPRTNGMCSVCYKEHLQRQNSSNGRISPPAPSVTSLSESLPVQCTDGSVPEAQSALDSTASSVQPSPVSNQSLLSESVASSQVDSTSVDKAIPETEDLQASVSETAQQASEEQSKSLEKPKQKKNRCFMCRKKVGLTGFECRCGNVYCGVHRYSDVHNCSYNYKADAAEKIRKENPVVVGEKIQKI

SEQ ID NO: 207 - protein, 70 aa, Bovine
  VARIANT (1)...(70): Xaa = Any Amino Acid
NIPAGTTVDTKITHPTEFDFYLCSHAGIQGTSRPSHYHVLWDDNRFSSDELQILTYQLXHTYVRCTRSVV

SEQ ID NO: 208 - protein, 60 aa, Bovine
  VARIANT (1)...(60): Xaa = Any Amino Acid
ALLDVQFRNTTIGLTVFAIKKYVVFLRLFLETAEKYFMXGHKVIYYVFTDRPADVPQIAL

SEQ ID NO: 209 - protein, 124 aa, Bovine; identical to SEQ ID NO: 139

SEQ ID NO: 210 - protein, 107 aa, Bovine
DFGTMKDKIAANEYKSVTEFKADFKLMCDNAMTYNRPDTVYYKLAKKILHAGFKMMSKERLLALKRSMSFMQDMDFSQQAALLGNEDTAAEEPVPEVVPVHVETAKK

SEQ ID NO: 211 - protein, 150 aa, Bovine
QDLNSTAAPHPRLSQYKSKYSSLEQSERRRQLLELQKLKRLDYVNHARRLAEDDWTGMESEEEEEKKDDEEMDVDTGKELPKRYANQLMLSEWLIDVPSDLGQEWIVVVCPVGKRSLIVASQGLTSAYTRSGYWVNTFPSLLPGGNRRNS

SEQ ID NO: 212 - protein, 124 aa, Bovine
IQELRRGSQAANIYCINFNQDASLICVSSDHGTVHIFAAEDPKRNKQSSLASASFLPKYFSSKWSFSKFQVPSGSPCICAFGTEPNAVIAICADGSYYKFLFNPKGECVRDVYAQFLEMTDDKL

SEQ ID NO: 213 - protein, 75 aa, Bovine
DCGLDSCYNSSGALQFLQKNSSKYHFRRTKMLPVSGGFHTRLMEPAVEPLVQVLKAIDVKKPLVSVHSNVDGNKY

SEQ ID NO: 214 - protein, 108 aa, Bovine
CDVPAKAIASALHGLCAQILSERVEVSGDSPCCSLDPITPEDLPRQVELLDAVSQAAQKYEALYMGTLPVTKAMGMDVLNEAIGRGWCRGGTTVAVSCAPRDLYWCWS

SEQ ID NO: 215 - protein, 67 aa, Bovine
MGVEGCTKCIKYLLFVFNFVFWLAGGVILGVALWLRHDPQTTNLLYLELGDRPAPNTFYVGIYILIA

SEQ ID NO: 216 - protein, 76 aa, Bovine
IFLGSKITADGDCSHEIERCFLLGRKLMTNLDSILKSRDITLPTKVHPVEAMVFPVVMYGCESWTIKKAEYRRIDS

SEQ ID NO: 217 - protein, 159 aa, Bovine
DVPHPPLKIPGGRGNSQRDHNLSANLFYSDNRLNVTEELTSNNKTRIFNVQSRLTEAKHINWRAVLSNSCLYVEIPGGALPEGSKDSFAVLLEFAEEQLHVDHVFICFHKNRDDRAALLRTFSFLGFEIVRPGHPLVPKRPDACFMAYTFERESSGEEE

SEQ ID NO: 218 - protein, 117 aa, Bovine
RKRRSDPNFKNRLRERRKKQKLAKERAGLSKLPDLKDAEAVQKFFLEEIQLGEELLAQGEYEKGVDHLTNAIAVCGQPQQLLQVLQQTLPPPVFQMLLTKLPTISQRIVSAQSLAER

SEQ ID NO: 219 - protein, 148 aa, Bovine
MRKFAYCKVVLATSLIWVLLDMFLLLYFSECNKCDEKKERGLPAGDVLEPVQKPHEGPGEMGKPVVIPKEDQEKMKEMFKINQFNLMASEMIALNRSLPDVRLEGCKTKVYPDNLPTTSVVIVFHNEAWSTLLRTVHSVINRSPKHML

SEQ ID NO: 220 - protein, 127 aa, Bovine
RVPGRHGYAAEFSPYLPGRLACAASQHYGIAGSGTLLILDQNESGLRLFRSFDWNDGLFDVTWSENNEHVLVTCSGDGSLQLWDTARATGPLQVFKEHTQEVYSVDWSQTRGEQLVVSGSWDQTVKL

SEQ ID NO: 221 - protein, 100 aa, Bovine
MDESALTLGTIDVSYLPNSSEYSIGRCKHATEEWGECGSRPTVFRSATLKWKESLMSRKRPFVGRCCYSCTPQSWDKFFNPSIPSLGLRNVIYINETHTR

SEQ ID NO: 222 - protein, 200 aa, Bovine
MANGYTYEDYQDTAKWLLSHTEQRPQVAVICGSGLGGLVNKLTQAQTFDYSEIPNFPESTVPGHAGRLVFGILNGRACVMMQGRFHMYEGYPFWKVTFPVRVFRLLGVETLVVTNAAGGLNPNFEVGDIMLIRDHINLPGFSGENPLRGPNEERFGVRFPAMSDAYDRDMRQKAHSTWKQMGEQRELQEGTYVMLGGPNF

SEQ ID NO: 223 - protein, 157 aa, Bovine
QSEPLTGVFTTEEVPAQQYLEIDEVTPDSFRVSWHPLSADEGQHKLMWIPVYGGSTEEVVLQEDQDSYVIEGLEPGTEYEVSLLAVLDDGSESEVVTAVGTTLDSFWTEPPTTEEAPTRPVTSVFRTGIRNLVVDAETTSSLRVAWDISNSSVQAIQ

SEQ ID NO: 224 - protein, 128 aa, Bovine
RSKCYTFKGPGNRPLPRMEGRNFSPVPSKPRSQSPGEEENSLNEDWYVSYVTRTEAEAALRKINQDGTFLVRDSSKKTISNPYVLMVLYKDKVYNIQIRYQEESQVYLLGTGLRGKEDFLSVSDIIDY

SEQ ID NO: 225 - protein, 187 aa, Bovine
ASARKAAQVTIQSSGTFSTKFQVENSNRLLLQQVSLPEVPGEYCMSVTGEGCVYLQTSLKYNILPKKDEFPFALEVQTLPQTCDGPKAHTSFQISLSVSYIGSRPASNMAIVDVKMVSGFIPLKPTVKMLERSNVSRTEVSNNHVLIYLDKVTNETLTLTFTVLQDIPVRDLKPAIVKVYDYYETDE

SEQ ID NO: 226 - protein, 184 aa, Bovine
DHLEAKKPLSTPSLTTEDWLVQNHQDPYKVEEVCKANEPCTSFAECVCDENCEKEALCKWLLKKEGKDKNGMPVDPKPEPGKHKDSLNTWLSPSGREAAEQARAPQATAAGVADSFQVIRSSPLSEWLMTPSHKEGCPNKEAPLTEDRASKQKLTSPLATAWCPFNTADWVLPAKKTGNLSQLS

SEQ ID NO: 227 - protein, 161 aa, Bovine
ESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETIIIALASVSVLAVLIVALCFGYRMLTGDRKQGLHSMNMMEAAASEPSLDLNNLKLLEL

SEQ ID NO: 228 - protein, 86 aa, Bovine
EKRAYLQSRFPQLNETSFANSRDTSFEQHVLWHTAGKGADLVLNSLAEEKLQASVRCLAQHGRFLEIGKFDLSKNHPLGAGHPPYL

SEQ ID NO: 229 - protein, 75 aa, Bovine
VNAAGGPTPSQRGLSDLALCGPAANQCAGPAKDRVDCGYPEVTPEQCNNRGCCFDSSIHGVPWCFKPLQEAECTF

SEQ ID NO: 230 - protein, 77 aa, Bovine
SGPTSEKPARSHPWTPDDSTDTNGSDNSIPMAYLTLDHQLQPLAPCPNSKESMAVFEQHCKMAQEYMKVQTEIALLL

SEQ ID NO: 231 - protein, 112 aa, Bovine
PIILVGNKSDLVRSREVSLDEGRACAVVFDCKFIETSAALHHNVQALFEGVVRQIRLRRDSKEANARRQAGTRRRESLGKKAKRFLGRIVARNSRKMAMRAKSKSCHDLSVL

SEQ ID NO: 232 - protein, 167 aa, Bovine
CFVASILLLAVARCILFLIIWLITGGRHHFWFLPNLTADVGFIDSFRPLYTHEYKGPKADLKKDEKSETKKQQKSDSEEKSDSEKKEDEEGKVGPGNHGTEGSGGERHSDTDSDRREDDRSQHSSGNGNDFEMITKEELEQQTDGDCEEEEEEDNDGETTKSSHEKS

SEQ ID NO: 233 - protein, 106 aa, Bovine
CEGPEEESEDDPQLEGRDPDIWHVGFKISWDIETPGLAIPLHQGDCYFMLDDLNATHQHCVLAGLPPRFSSTHRVAECSTGTLEYILQRCQVALQNVREEADNGEI

SEQ ID NO: 234 - protein, 126 aa, Bovine
MLMVLCPPLAWAREIQPHFLEYSTSECHFFNGTERVRFLDRYFHNGEEFVRFDSDWGEYRAVTELGRPDAEYWNSQEILERARAAVDTYCRHNYGGVESFTVQRRVEPTVTVYPAKTQPLQHHNLL

SEQ ID NO: 235 - protein, 170 aa, Bovine
HELTLAEYHEQEEIFKLRLGHLKKEEAEIQAELERLERVRNLHIRELKRIHNEDNSQFKDHPTLNDRYLLLHLLGRGGFSEVYKAFDLTEQRYVAVKIHQLNKNWRDEKKENYHKHACREYRIHKELDHPRIVKLYDYFSLDTDSFCTVLEYCEGNDLDFYLKQHKLMSE

SEQ ID NO: 236 - protein, 228 aa, Bovine
MLDSVTHSTFLPNTSFCDPLMSWTDLFSNEEYYPAFEHQTACDSYWTSVHPEYWTKRHVWEWLQFCCDQYKLDANCISFCHFNISGLQLCGMTQEEFMERPASVGSICTLSSRASAHKVTPFLMILMRPRPSLQSSHLWEFVRDLLLSPEENCGILEWEAREQGIFRVVKSEALAKMWGQRKKNDRMTYEKLSRALRYYYKTGILERVDRRLVYKFGKNAHGWQEDKL

SEQ ID NO: 237 - protein, 120 aa, Bovine
DTKGFCSANLLEDLPLQEPQSPHKLNAGFDLAKGGAGKVNLPKELAADAVNILPASLDLSPLLGFWQLPPATQNAFGSSGLAWGLGNLCRIGWAVWGSKPQDPSLAMSTMSLGQLPLHPS

SEQ ID NO: 238 - protein, 314 aa, Bovine
MTEQMTLRGTLKGHNGWVTQIATTPQFPDMILSASRDKTIIMWKLTRDETNYGIPQRALRGHSHFVSDVVISSDGQFALSGSWDGTLRLWDLTTGTTTRRFVGHTKDVLSVAFSSDNRQIVSGSRDKTIKLWNTLGVCKYTVQDESHSEWVSCVRFSPNSSNPIIVSCGWDKLVKVWNLANCKLKTNHIGHTGYLNTVTVSPDGSLCASGGKDGQAMLWDLNEGKHLYTLDGGDIINALCFSPNRYWLCAATGPSIKIWDLEGKIIVDELKQEVISTSSKAEPPQCTSLAWSADGQTLFAGYTDNLVRVWQVPS

SEQ ID NO: 239 - protein, 116 aa, Bovine
YYTTPIYRFRMKCHLCVNYIEMQTDPANCDYVIVSGAQRKEERWDMEDNEQVLTTEHEKKQKLEMDAMFRLEHGEADRSTLKKALPTLSHIQEAQSAWKDDFALNSMLRKRFREKK

SEQ ID NO: 240 - protein, 166 aa, Bovine
LTGPGRTEVGKNSEKKVESEENVNQDRNQDNEDIGDSKDIRLTLMEEVLLLGLKDKEGYTSFWNDCISSGLRGGILIELAMRGRIYLEPPTMRKKRLLDRKVLLKSDSPTGDVLLDETLKHIKAIEPTETVQTWIELLTGETWNPFKLQYQLRNVRKRIAKPSREG

SEQ ID NO: 241 - protein, 148 aa, Bovine
MEKHLFNLKFAAKELGRSAKKCDKEEKAEKAKIKKAIQKGNMEVARIHAENAIRQKNQAVNFLRMSARVDAVAARVQTAVTMGKVTKSMAGVVKSMDATLKTMNLEKISALMDKFEHQFETLDVQTQQMEDTMSSTTTLTTPQGQVDM

SEQ ID NO: 242 - protein, 49 aa, Bovine
PCRLDCYGGLIECYLASNSIREAMVMANNVYKTLGANAQTLTLLATVCL

SEQ ID NO: 243 - protein, 98 aa, Bovine
MVKVTFNSALAQKEAKKDESKSGEEALIIPPDAVAVDCKDPDEVVPVGQRRAWCWCMCFGLAFMLAGVILGGAYLYKYFAFQPDDVYYCGIKYIKDDV

SEQ ID NO: 244 - protein, 352 aa, Bovine
EQNKLLETKWALLQEQKSAKSNRLPGIFEAQIAGLRKQLEALQLDGGRLEVELRNMQDVVEDFKNKYEDEINHRTAAENEFVVLKKDVDVAYMNKVELEAKVDTLNDEINFLRTLYEQELKELQSEVSDTSVVLSMDNNRSLDLDSIIAEVKAQYEEIANRSRAEAEACYQTKFETLQAQAGKHGDDLRNTRNEIADMNRAVQRLQAEIDSVKNQRSKLEAAIADAEQRGELAVKDARAKQEDLEAALQKAKQDMTRQLREYQELMNVKLALDIEIATYRKLLEGEESRLTGDGVGAVNISVVSSTGGSGSLLTFGGTMGNNALRFSSGGGPGTLKAYSMRTTSATSRSPRK

SEQ ID NO: 245 - protein, 99 aa, Bovine
RVLGERQRKEEEMKQLFVQRVKEKEAILKEAERELQAKFEHLKRVHQEEKLRLEEKRRLLEEEIMAFSKKKATSEIYQNQTFMTPGSNLRKDKDRKNSN

SEQ ID NO: 246 - protein, 58 aa, Bovine
FVSPEHVKHCFWLTQEFRYLSQTHTNHEDKLQVKNVIYHAVKDAVAMLKASESSFGKP

SEQ ID NO: 247 - protein, 91 aa, Bovine
KHLDVDLDRQSLSSIDKNASERGQSQLSNPTDDGWKARPYANQKLFASLLIKCVVQLELIQTIDNIVFYPATSKREDAEHMAAMPQPVPTA

SEQ ID NO: 248 - protein, 86 aa, Bovine
REYHITVDEPRLKQPPSGFDSVIARGHTEPDPTRDTELELDGQRVVVPQGQPVLCPDFRSCNFSQSEYLIYQESQRCLRYLLEIHL

SEQ ID NO: 249 - protein, 138 aa, Bovine
LSKISHAKPAIADYAFTTIKPELGKIMYSDFKQISVADLPGLIEGAHMNKGMGHKFLKHIERTKQLLFVVDISGFQLSSQTHYRTAFETIILLSKELELYKEELHTKPALLAVNKMDLPDAQGKFHVLMNQLQNSKEF

SEQ ID NO: 250 - protein, 85 aa, Bovine
KPWDDETDMAKLEECVRSIQADGLVWGSSKLVPVGYGIKKLQIQCVVEDDKVGTDMLEEQITAFDEYVQSMDGRLGDKCWFFGFL

SEQ ID NO: 251 - protein, 112 aa, Bovine; identical to SEQ ID NO: 231

SEQ ID NO: 252 - protein, 111 aa, Bovine
QKCSKQHSEIRENLITALSTWQMFIVDIKRNNTAFDIIADNCDLHFKISRDRLSASSLTMESFAFLWAGGRASYGVSKGKVCFEMKVTEKIPVRHLYTKDIDIMKFGLGGH

SEQ ID NO: 253 - protein, 166 aa, Bovine
YFVTDYDPTIEDSYTKQCVIDDRAARLDILDTAGQEEFGAMREQYMRTGEGFLLVSSVTDRGSFEEIYKFQRQILRVKDRDEFPMILIGNKADLDHQRQVTQEEGQQLARQLKVTYMEASAKIRMNVDQAFHELVRVIRKFQEQECPPSPEPTRKGKRQERLHCVI

SEQ ID NO: 254 - protein, 76 aa, Bovine
MSKAHPPELKKFMDKKLSLKLNGGRHVQGILRGFDPFMNLVIDECVEMATSGQQNNIGMVVIRGNSIIMLEALERV

SEQ ID NO: 255 - protein, 161 aa, Bovine
  VARIANT (1)...(161): Xaa = Any Amino Acid
MAARRDGWLGPAFGLRLLLATVLQTVSALGAEFSSESCRELGFSSNLLCSSCDLLGQFNLLQLDPDCRGCCQEEAQFETKKLYAGAILEVCXKLGRFPQVQAFVRSDKPKLFKGLQIKYVRGSDPVLKLLDDSGNIAEELSILKWNTDSVEEFLSEKLERI

SEQ ID NO: 256 - protein, 94 aa, Bovine
KTDMFQTVDLFEGKDLAAVQRTLMALGSLAVTKNDGHYRGDPNWFMKKAQEHKREFTESQLQEGKHVIGLQMGSNRGASQAGMTGYGRPRQIIS

SEQ ID NO: 257 - protein, 101 aa, Bovine
VPTMVTRGQDVGRYQVSWSLDHKSAHAGTYEVRFFDEESYSLLRKAQRNNEDVSVIPPLFTVSVDHRGTWNGPWVSTEVLAAAIGLVIYYLAFSAKSHIQA

SEQ ID NO: 258 - protein, 105 aa, Bovine
SFRDIYFDTLNEDLFQKILVPIQQVLKEGHLEKTEIDEVVLVGGSTRIPRIRQVIQEFFGKDPNTSVDPDLAVVTGVAIQAGIDGGSWPLQVSALEIPNKHLQKT

SEQ ID NO: 259 - protein, 128 aa, Bovine
GTWDSFLEKFMAGEVCYGSWYQHVHEWWELSHTHPVLYLFYEDIMEDPKREIQKILEFIGRSLPEETVDHIVQRPYPLQSWTTSISSFMRKGITGDWKSTFTVAQNELFEAHYAKKMRAASFRFRWKL

SEQ ID NO: 260 - protein, 76 aa, Bovine
QKKASASAGRITVPRLSVGSVTSRPSTPTLGTPTPPAMSVSTKVGTPVSLTGQRFTVQMPTSQSPAVKASIPATSA

SEQ ID NO: 261 - protein, 169 aa, Bovine
MAAVKTLNPKAEVARAQAALAVNISAARGLQDVLRTNLGPKGTMKMLVSGAGDIKLTKDGNVLLHEMQIQHPTASLIAKVATAQDDITGDGTTSNVLIIGELLKQADLYISEGLHPRIITEGFEAAKEKALQFLEQVKVSKEMDRETLIDVARTSLRTKVHAELADVLT

SEQ ID NO: 262 - protein, 198 aa, Bovine
KMSDMEDDFMCDDEEDYDLEYSEDSNSEPNVDLENQYYNSKALKEDDPKAALSSFQKVLELEGEKGEWGFKALKQMIKINFKLTNFPEMMNRYKQLLTYIRSAVTRNYSEKSINSILDYISTSKQNSDFLCQMDLLQEFYETTLEALKDAKNDTLWFKTNTKLGKLYLEREEYGKLQKILRQLHQSCQTDDGEDDLKK

(19) United States
(12) Patent Application Publication
(10) Pub. No.: US 2005/0130263 A1
(43) Pub. Date: Jun. 16, 2005

(54) Compositions isolated from bovine mammary gland and methods for their use

(75) Inventors: Ilkka J. Havukkala, Remura (NZ); Matthew Glenn, Whenuapai (NZ); Murray R. Grigor, Glendowie (NZ); Adrian J. Molenaar, Hamilton (NZ)

Correspondence Address: Janet Sleath;SPECKMAN LAW GROUP, Suite 100, 1501 Western Avenue, Seattle, WA, 98101, US

(73) Assignees: GENESIS RESEARCH AND DEVELOPMENT CORP. LTD., Parnell (NZ); AGRESEARCH LTD., Hamilton (NZ)

(21) Appl. No.: 10/617,316

(22) Filed: Jul. 9, 2003

Related U.S. Application Data
(63) Continuation-in-part of application No. 09/699,146, filed on Oct. 27, 2000, now abandoned.
(60) Provisional application No. 60/162,702, filed on Oct. 29, 1999.

(51) Int. Cl. C12Q 1/68; C07H 21/04; C07K 14/76; C07K 14/47; A61K 38/24

(52) U.S. Cl. 435/69.1; 435/6; 435/320.1; 435/325; 530/360; 530/362; 530/399; 536/23.5

(57) ABSTRACT

Isolated polynucleotides encoding polypeptides expressed in bovine mammary gland tissue are provided, together with genetic constructs and host cells comprising such isolated polynucleotides. Methods for the use of such polynucleotides and polypeptides are also provided.

### CROSS-REFERENCE TO RELATED APPLICATIONS

This application is a continuation-in-part of U.S. patent application Ser. No. 09/699,146, filed Oct. 27, 2000 which claims priority under 35 U.S.C. §119(e) to U.S. Provisional Patent Application No. 60/162,702, filed Oct. 29, 1999.

### TECHNICAL FIELD OF THE INVENTION

This invention relates to polypeptides expressed in bovine mammary gland cells, polynucleotides encoding such polypeptides, and methods for using the polypeptides and polynucleotides.

### BACKGROUND OF THE INVENTION

The bovine mammary gland is a milk-producing organ of great economic importance. Knowledge of the genes expressed in this tissue is valuable in understanding the physiology and function of the mammary gland, not only in the cow, but also in other mammals, including humans. The polynucleotide and polypeptide sequences themselves are useful in a wide variety of applications, which are described in greater detail below.

### SUMMARY OF THE INVENTION

The present invention provides isolated polypeptides expressed in bovine mammary gland cells and isolated polynucleotides encoding such polypeptides, together with expression vectors and host cells comprising such polynucleotides. Methods for using such polypeptides, polynucleotides and expression vectors are also provided.

In specific embodiments, isolated polynucleotides are provided that comprise a polynucleotide sequence selected from the group consisting of: (a) sequences recited in SEQ ID NOS: 1-131; (b) complements of the sequences recited in SEQ ID NOS: 1-131; (c) reverse complements of the sequences recited in SEQ ID NOS: 1-131; (d) reverse sequences of the sequences recited in SEQ ID NOS: 1-131; and (e) sequences having at least 75%, 90%, 95% or 98% identity to a sequence of (a)-(d), the percentage identity being determined as described below. Polynucleotides comprising at least a specified number of contiguous residues (“x-mers”) of any of the sequences identified as SEQ ID NOS: 1-131 are also provided, together with extended sequences, and oligonucleotide probes and primers corresponding to the sequences set out in SEQ ID NOS: 1-131. All of the polynucleotides described above, and oligonucleotide probes and primers, are collectively referred to herein as “polynucleotides of the present invention”.

In further embodiments, the present invention provides isolated polypeptides comprising an amino acid sequence encoded by a polynucleotide comprising: (a) a sequence provided in SEQ ID NOS: 1-131; or (b) a sequence having at least 75%, 90%, 95% or 98% identity to a sequence provided in SEQ ID NOS: 1-131, together with isolated polynucleotides encoding such polypeptides. In certain specific embodiments, the inventive polypeptides comprise an amino acid sequence selected from the group consisting of sequences identified as SEQ ID NOS: 132-262, and variants thereof. Isolated polypeptides comprising at least a functional portion of a polypeptide comprising an amino acid sequence selected from the group consisting of sequences identified as SEQ ID NOS: 132-262 and variants thereof, are also provided.

In related embodiments, the present invention provides genetic constructs, or expressions vectors, comprising the above polynucleotides, together with host cells transformed with such constructs, and organisms comprising such host cells.

In a further aspect, the present invention provides methods for stimulating bovine mammary gland cell growth and function, inhibiting the growth of various mammary gland cancer cells, inhibiting angiogenesis and vascularization of tumors, or modulating the growth of blood vessels in a mammal, such methods comprising administering to the subject a composition comprising an isolated polypeptide of the present invention. Methods for modulating mammary gland function in a mammal are also provided, the methods comprising administering to the subject a composition comprising an inventive polypeptide. Numerous utilities for the polynucleotides and polypeptides are described in greater detail below.

As detailed below, the isolated polynucleotides and polypeptides of the present invention may be usefully employed in the preparation of therapeutic agents for the treatment of mammary gland and other types of disorders. In addition, polynucleotides that are specifically expressed at a higher or lower level in diseased mammary gland than in a normal mammary gland may be used as an indicator of the disease condition. Similarly, disposition to a disease related to a specific level of expression of a polynucleotide would suggest use of that polynucleotide as a marker for diagnosis of susceptible individuals. In yet another aspect, the mapping of a specific polynucleotide of this invention close to the chromosomal location of any beneficial or detrimental genes would make the polynucleotide a valuable tool for breeding of livestock, disease diagnostics, or identification of the beneficial or detrimental gene.

The isolated polynucleotides of the present invention, have further utility in genome mapping, in physical mapping, and in positional cloning of genes. Additionally, the polynucleotide sequences identified as SEQ ID NOS: 1-131, and their variants, may be used to design oligonucleotide probes and primers (referred to collectively as “oligonucleotides”). As detailed below, oligonucleotide probes and primers have sequences that are substantially complementary to the polynucleotide of interest over a certain portion of the polynucleotide. The inventive oligonucleotide probes may be used to detect the presence, and examine the expression patterns, of genes in any organism having sufficiently similar DNA and RNA sequences in their cells using techniques that are well known in the art, such as slot blot DNA hybridization techniques. The inventive oligonucleotide primers may be used for PCR amplifications. Oligonucleotide probes and primers of the present invention may also be used in connection with various microarray technologies, including the microarray technology of Affymetrix, Inc. (Santa Clara, Calif.).

The above-mentioned and additional features of the present invention, together with the manner of obtaining them; will be best understood by reference to the following more detailed description. All references referred to herein are incorporated herein by reference in their entirety as if each was incorporated individually.

### DETAILED DESCRIPTION OF THE INVENTION

In certain aspects, the present invention provides polynucleotides that were isolated from cDNA libraries prepared from bovine mammary gland cells, together with polypeptides encoded by such polynucleotides.

The polynucleotides of the present invention encode polypeptides that have important roles in growth, development and function of mammary gland cells, and in responses of mammary gland cells to tissue injury and inflammation, as well as disease states. Many of the polypeptides disclosed herein have antibacterial or other bioactive utility. The polypeptides and/or polynucleotides of the present invention may be employed in the modification of mammary function, as potential markers for selection of livestock having enhanced mammary performance, and as diagnostics for abnormal cellular growth in mammary cancer. Oligonucleotide probes and primers corresponding to the polynucleotides of the present invention may be employed to detect the presence of mammary gland tissue in a specific tissue sample using techniques well known in the art, such as DNA hybridization and polymerase chain reaction (PCR) amplification.

The inventive polypeptides have important roles in processes such as induction of mammary growth differentiation of milk producing cells, cell migration, cell proliferation, and cell-cell interaction. The polypeptides are important in the maintenance of tissue integrity, and thus are important in processes such as wound healing. Some of the disclosed polypeptides modulate immune responses, and some of the polypeptides which are present in milk are immunologically active polypeptides that benefit mammalian offspring. In addition, many of the polypeptides are immunologically active within the mammary gland, making them important therapeutic targets for treating a whole range of disease states not only within the mammary gland, but also in other tissues of a mammal. Antibodies to the polypeptides of the present invention and small molecule inhibitors related to the polypeptides of the present invention may also be used for modulating immune responses and for treatment of diseases according to the present invention.

The correspondence of isolated polynucleotides encoding isolated polypeptides of the present invention, and the functionality of the polypeptides, are shown, below, in Table 1.

TABLE 1

| | SEQ ID | | |
|---|---|---|---|
| SEQ ID | NO: | | |
| NO: | Amino | | |
| DNA | acid | Activity Category | Functionality |
| 1 | 132 | Cell signaling, | Connective tissue growth factor is the major |
| | | extracellular factor, | connective tissue mitoattractant secreted by human |
| | | hormone | vascular endothelial cells. This immediate-early |
| | | | protein may bind one of the PDGF cell surface |
| | | | receptors. It belongs to the insulin-like growth |
| | | | factor binding protein family. |
| 2 | 133 | Gene/protein | Splicing factor, arginine/serine-rich 2, also known |
| | | expression, RNA | as splicing factor SC35, splicing component 35 kDa, |
| | | synthesis, RNA | or PR264 protein, interacts with spliceosomal |
| | | processing. | components bound to both the 5′ and 3′ splice sites |
| | | | during spliceosome assembly. It also is required for |
| | | | ATP-dependent interactions of both U1 and U2 |
| | | | snRNPS with pre-mRNA. |
| 3 | 134 | Metabolism, | GTP cyclohydrolase I feedback regulatory protein |
| | | cofactor, synthesis | mediates tetrahydrobiopterin inhibition of GTP |
| | | | cyclohydrolase I, the initial enzyme of the de novo |
| | | | pathway for biosynthesis of tetrahydrobiopterin, |
| | | | the cofactor required for aromatic amino acid |
| | | | hydroxylations and nitric oxide synthesis. This |
| | | | inhibition is reversed by L-phenylalanine. High |
| | | | expression in liver and kidney and lower level in |
| | | | testis, heart, brain and lung. |
| 4; 5; 105 | 135; 136; | Gene/protein | Ets transcription factor 2 is a member of the Ets |
| | 236 | expression, RNA | transcription factor family that binds to specific |
| | | synthesis, | sites in the promoters of genes to activate them. |
| | | transcription factors | |
| 6 | 137 | | Ancient ubiquitous protein isoform 1 (AUP1) is a |
| | | | conserved protein that is ubiquitously expressed |
| | | | across many tissues. |
| 7 | 138 | Gene/protein | Pantophysin is a homolog of the integral membrane |
| | | expression, | protein synaptophysin and is one of the major |
| | | membrane protein | polypeptide components of the small, electron- |
| | | | translucent, transmitter-containing vesicles in |
| | | | neurons and of similar vesicles in neuroendocrine |
| | | | (NE) cells. In contrast to synaptophysin, |
| | | | pantophysin is ubiquitously expressed. |
| 8 | 139 | Cell signaling, | MEK binding partner 1, is a MEK binding partner |
| | | protein modification, | that enhances enzymatic activation of the MAP |
| | | kinase | kinase cascade. |
| 9 | 140 | Gene/protein | NDRG1 protein, also known as n-MYC |
| | | expression, RNA | downstream regulated gene 1 protein, |
| | | synthesis, | differentiation-related gene 1 protein (DRG1), |
| | | transcription factors | reducing agents and tunicamycin-responsive, |
| | | | protein, RTP, nickel-specific induction protein, |
| | | | CAP43, RIT42, may have a growth inhibitory role. |
| | | | It is located in both the cytoplasm and the nucleus |
| | | | in prostate epithelium and placental chorion, |
| | | | although nuclear staining is not observed in colon |
| | | | epithelium cells. Instead its localization changes |
| | | | from the cytoplasm to the plasma membrane during |
| | | | differentiation of colon carcinoma cell lines in |
| | | | vitro. It is ubiquitously expressed, most |
| | | | prominently in placental membranes and prostate, |
| | | | kidney, small intestine, and ovary tissues. Reduced |
| | | | expression in adenocarcinomas compared to |
| | | | normal tissues. In colon, prostate and placental |
| | | | membranes, the cells that border the lumen show |
| | | | the highest expression. It is induced by |
| | | | homocysteine, 2-mercaptoethanol, tunicamycin in |
| | | | endothelial cells and by nickel compounds in all |
| | | | tested cell lines. The primary signal for its |
| | | | induction is an elevation of free intracellular |
| | | | calcium ion caused by nickel ion exposure. |
| | | | Okadaic acid, a serine/threonine phosphatase |
| | | | inhibitor, induced expression of NDRG1 more |
| | | | rapidly and more efficiently than nickel. |
| 10 | 141 | Cell/organism | Protein HSPC030, is a member of the heat shock |
| | | defense, homeostasis, | induced family of proteins. |
| | | stress response | |
| 11 | 142 | Cell/organism | HSPC203, is a member of the heat shock induced |
| | | defense, homeostasis, | family of proteins obtained as a full-length cDNA |
| | | stress response | cloned from CD34+ stem cells. |
| 12 | 143 | Cell | Actins are highly conserved proteins that are |
| | | structure/motility, | involved in various types of cell motility and are |
| | | cytoskeletal | ubiquitously expressed in all eukaryotic cells. |
| | | | Polymerization of globular actin (G-actin) leads to |
| | | | a structural filament (F-actin) in the form of a two- |
| | | | stranded helix. Each actin can bind to four others. |
| | | | In vertebrates, three main groups of actin isoforms |
| | | | (alpha, beta and gamma) have been identified. The |
| | | | alpha actins are found in muscle tissues and are a |
| | | | major constituent of the contractile apparatus. The |
| | | | beta and gamma actins co-exist in most cell types |
| | | | as components of the cytoskeleton and as mediators |
| | | | of internal cell motility. |
| 13 | 144 | Cell/organism | Ig alpha-2 chain C region contains the alpha chain |
| | | defense, | of the immunoglobulin A (IgA, immunoglobulin |
| | | immunology, | alpha) class of antibodies. Ig alpha is the major |
| | | antibody | immunoglobulin class in body secretions. It serves |
| | | | both to defend against local infection and to |
| | | | prevent access of foreign antigens to the general |
| | | | immunologic system. |
| 14 | 145 | Cell signaling, | Angiogenin-1, may function as a tRNA-specific |
| | | extracellular factor, | ribonuclease that binds to actin on the surface of |
| | | cytokine | endothelial cells; once bound, angiogenin is |
| | | | endocytozed and translocated to the nucleus, |
| | | | thereby promoting the endothelial invasiveness |
| | | | necessary for blood vessel formation. Angiogenin |
| | | | induces vascularization of normal and malignant |
| | | | tissues and abolishes protein synthesis by |
| | | | specifically hydrolyzing cellular tRNAs. Binds |
| | | | tightly to placental ribonuclease inhibitor and has |
| | | | very low ribonuclease activity. |
| 15 | 146 | Metabolism, lipid, | Clathrin coat assembly protein AP19, also known |
| | | intracellular transport | as clathrin coat associated protein AP19, Golgi |
| | | | adaptor AP-1 19 kDa adaptin, HA1 19 kDa |
| | | | subunit, or clathrin assembly protein complex 1 |
| | | | small chain, is a component of the adaptor |
| | | | complexes which link clathrin to receptors in |
| | | | coated vesicles. Clathrin-associated protein |
| | | | complexes are believed to interact with the |
| | | | cytoplasmic tails of membrane proteins, leading to |
| | | | their selection and concentration. AP19 is a subunit |
| | | | of the Golgi membrane adaptor. Assembly protein |
| | | | complex 1 (AP-1) is a heterotetramer composed of |
| | | | two large chains (gamma and beta), a medium |
| | | | chain (AP47) and a small chain (AP19). |
| 16 | 147 | Metabolism, lipid, | Clathrin coat assembly protein AP50, also known |
| | | intracellular transport | as clathrin coat associated protein AP50, plasma |
| | | | membrane adaptor AP-2 50 kDa protein, HA2 50 kDa |
| | | | subunit, clathrin assembly protein complex 2 |
| | | | medium, chain, AP-2 mu 2 chain, is a component |
| | | | of the adaptor complexes which link clathrin to |
| | | | receptors in coated vesicles. Clathrin-associated |
| | | | protein complexes are believed to interact with the |
| | | | cytoplasmic tails of membrane proteins, leading to |
| | | | their selection and concentration. AP50 is a subunit |
| | | | of the plasma membrane adaptor. The assembly |
| | | | protein complex 2 (AP-2) is a heterotetramer |
| | | | composed of two large chains (alpha and beta), a |
| | | | medium chain (AP50) and a small chain (AP17). |
| | | | It is expressed in brain, heart, lung, liver, testis, and |
| | | | spleen. |
| 17 | 148 | Cell | ARP2/3 complex 34 kDa subunit is part of a |
| | | structure/motility, | complex implicated in the control of actin |
| | | cytoskeletal | polymerization in cells. The ARP2/3 protein |
| | | | complex has been implicated in the control of actin |
| | | | polymerization in cells. The human complex |
| | | | consists of seven subunits that include the actin- |
| | | | related proteins ARP2 and ARP3, and five others. |
| 18 | 149 | Metabolism, ATP | Calcium-transporting ATPase plasma membrane, |
| | | synthesis | isoform 1b is an integral membrane protein that |
| | | | catalyzes the hydrolysis of ATP coupled with the |
| | | | transport of the calcium. |
| 19 | 150 | Metabolism, ATP | ATP synthase A chain, is the key component of the |
| | | synthesis | proton channel; and plays a direct role in the |
| | | | translocation of protons across the membrane. |
| 20 | 151 | Metabolism, | ATP synthase E chain, mitochondrial, is one of the |
| | | transport, cellular | chains of the nonenzymatic component (CF(0) |
| | | uptake | subunit) of the mitochondrial ATPase complex. |
| 21 | 152 | Gene/protein | Retinoic acid receptor responder protein 2, also |
| | | expression, RNA | known as tazarotene-induced gene 2 protein, RAR- |
| | | synthesis, | responsive protein TIG2, highly expressed in skin |
| | | transcription factors | (basal and suprabasal layers of the epidermis, hair |
| | | | follicles and endothelial cells). Also found in |
| | | | pancreas, liver, spleen, prostate, ovary, small |
| | | | intestine and colon. Its induction is inhibited in |
| | | | psoriatic lesions. Activated by tazarotene in skin |
| | | | grafts and in the epidermis of psoriatic lesions. |
| 22 | 153 | Cell signaling, | Bovine leukemia virus cell receptor, also known as |
| | | receptors, membrane | BLV-r, BLVPCP1, is a type I membrane protein |
| | | | that binds to the envelope glycoprotein gp51 of the |
| | | | bovine leukemia virus. |
| 23 | 154 | Cell signaling, | Butyrophilin is a type I membrane protein that |
| | | receptors, membrane | functions in the secretion of milk-fat droplets. It |
| | | | acts as a specific membrane-associated receptor for |
| | | | the association of cytoplasmic droplets with the |
| | | | apical plasma membrane. It appears to associate |
| | | | with xanthine dehydrogenase/oxidase and is |
| | | | specificity expressed in mammary tissue and |
| | | | secreted in association with the milk-fat-globule |
| | | | membrane during lactation. |
| 24 | 155 | Cell | Collagen alpha 1 (XV) chain belongs to the |
| | | structure/motility, | multiplexin family of collagens and is expressed |
| | | extracellular matrix | predominantly in internal organs such as adrenal |
| | | | gland, pancreas and kidney. Prolines at the third |
| | | | position of the tripeptide repeating unit (G-X-Y) |
| | | | are hydroxylated in some or all of the chains. |
| 25 | 156 | Gene/protein | 60S Ribosomal protein L10A (RPL10a) belongs to |
| | | expression, protein | the L1p family of ribosomal proteins. |
| | | synthesis, ribosomal | |
| | | proteins | |
| 26 | 157 | Gene/protein | RNA-binding protein regulatory subunit, DJ-1 |
| | | expression, RNA | protein or CAP1 protein is a nucleic acid binding |
| | | synthesis, RNA | protein that plays a role in the control of protein |
| | | processing | synthesis. |
| 27; 108 | 158; 239 | Cell | Myosin-IXA (MYO9A) is an unconventional |
| | | structure/motility, | myosin that is associated with Bardet-Biedl |
| | | cytoskeletal | Syndrome (BBS) a heterogeneous, autosomal |
| | | | recessive, disorder characterized by mental |
| | | | retardation, obesity, retinitis pigmentosa, |
| | | | syndactyly and/or polydactyly, short stature, and |
| | | | hypogenitalism |
| 28 | 159 | Gene/protein | CCR4-associated factor 1 is a ubiquitous |
| | | expression, RNA | transcription factor required for a diverse set of |
| | | synthesis, | processes. It is a component of the CCR4 complex |
| | | transcription factors | involved in the control of gene expression. |
| 29 | 160 | Gene/protein | Cathepsin S is a lysosomal thiol protease that has |
| | | expression, protein | similar substrate specificity to those of cathepsin L |
| | | degradation, protease | and cathepsin N. |
| 30 | 161 | Metabolism, | Caveolin-1. Caveolins are proteins associated with |
| | | transport, cellular | caveolae, which are plasma membrane |
| | | uptake | specializations that are important for normal signal |
| | | | transduction. |
| 31 | 162 | Cell signaling, | Cysteine-rich secretory protein-3, CRISP-3, |
| | | extracellular factor, | although originally described in the male genital |
| | | cytokine | tract, the cysteine-rich secretory proteins (CRISPs) |
| | | | are expressed in a variety of mammalian tissue and |
| | | | cell types. The proteins of the male genital tract |
| | | | have been observed associated to spermatozoa and |
| | | | are believed to play a role in mammalian |
| | | | fertilization. CRISP-3. is transcribed and expressed |
| | | | in the salivary gland, in the ampulla and the |
| | | | seminal vesicle. Believed to play a role in the |
| | | | innate immune system (Haendler et al. Jnl. Cell. |
| | | | Physiol. 178: 371-378 (1999); Udby et al. J. |
| | | | Leukoc. Biol. 72: 462-9 (2002)). |
| 32 | 163 | Cell signaling, | Connective tissue growth factor is the major |
| | | extracellular factor, | connective tissue mitoattractant secreted by human |
| | | hormone | vascular endothelial cells. This immediate-early |
| | | | protein may bind one of the PDGF cell surface |
| | | | receptors. It belongs to the insulin-like growth |
| | | | factor binding protein family. |
| 33 | 164 | Gene/protein | Peptidyl-prolyl cis-trans isomerase A, also known |
| | | expression, protein | as PPIase, rotamase, cyclophilin A, cyclosporin A- |
| | | synthesis, post- | binding protein, accelerates the folding of proteins |
| | | translational | by catalysing the cis-trans isomerization of proline |
| | | modification/targeting | imidic peptide bonds in oligopeptides. The protein |
| | | | binds and is inhibited by cyclosporin A. |
| 34 | 165 | Gene/protein | Pre-mRNA splicing factor RNA helicase also |
| | | expression, RNA | known as DEAH box protein 15 N or ATP- |
| | | synthesis, RNA | dependent RNA helicase number 46, is a pre- |
| | | processing | mRNA processing factor involved in disassembly |
| | | | of spliceosomes after the release of mature mRNA. |
| 35 | 166 | Gene/protein | DEAD-box protein 1 also known as DEAD-box |
| | | expression, RNA | protein-retinoblastoma, DBP-RB, is a member of a |
| | | synthesis, RNA | family of “DEAD” box RNA helicases. DEAD-box |
| | | processing | genes are found throughout evolution and encode |
| | | | RNA-binding proteins including eukaryotic |
| | | | initiation factor-4a, which is essential for protein |
| | | | translation, VASA, which is essential for germ line |
| | | | development, and a number of nuclear and |
| | | | mitochondrial RNA splicing factors. It is expressed |
| | | | with highest levels of transcription in 2 |
| | | | retinoblastoma cell lines and in tissues of |
| | | | neuroectodermal origin including the retina, brain, |
| | | | and spinal cord where it has an important role for |
| | | | this gene in control of cell growth and division. |
| 36 | 167 | Metabolism, | NRH dehydrogenase [quinone] 2, also known as |
| | | cofactor, synthesis | quinone reductase 2, QR2, serves as a quinone |
| | | | reductase in connection with conjugation reactions |
| | | | of hydroquinones involved in detoxication |
| | | | pathways as well as in biosynthetic processes such |
| | | | as the vitamin K-dependent gamma-carboxylation |
| | | | of glutamatyl residues in prothrombin synthesis. |
| | | | Two separate but homologous cytosolic quinone |
| | | | reductases have been identified. QR2 uses |
| | | | dihydronicotinamide riboside (NRH) rather than |
| | | | NAD(P)H as the electron donor. |
| 37 | 168 | Cell | Dynein heavy chain, cytosolic, DYHC, or MAP |
| | | structure/motility, | 1C, has ATPase activity. Cytoplasmic dynein acts |
| | | microtubule- | as a motor for the intracellular retrograde motility |
| | | associated | of vesicles and organelles along microtubules and |
| | | proteins/motors | is therefore important in cell signaling and uptake |
| | | | and secretion of proteins. The active protein |
| | | | consists of at least two heavy chains and a number |
| | | | of intermediate and light chains. |
| 38 | 169 | Gene/protein | Elongation factor 1-alpha 1, also known as |
| | | expression, protein | elongation factor TU, belongs to the GTP-binding |
| | | synthesis, translation | elongation factor family and promotes the GTP- |
| | | | dependent binding of aminoacyl-tRNA to the A- |
| | | | site of ribosomes during protein biosynthesis. It is |
| | | | reported to be expressed in brain, placenta, lung, |
| | | | liver, kidney, pancreas but barely detectable in |
| | | | heart and skeletal muscle |
| 39 | 170 | Gene/protein | Elongation factor 1-gamma, EF-1-gamma, is part |
| | | expression, protein | of the EF-1 complex that is composed of four |
| | | synthesis, translation | subunits: alpha, beta, delta, and gamma. The |
| | | | gamma subunit plays a role in anchoring the |
| | | | complex to other cellular components and is |
| | | | essential for protein synthesis. |
| 40 | 171 | Gene/protein | Protein disulfide isomerase ER-60, also known as |
| | | expression, protein | glucose-regulated protein ERp57/GRP58 is an |
| | | synthesis, post- | endoplasmic reticulum protein that catalyzes the |
| | | translational | rearrangement of both intrachain and interchain |
| | | modification/ | disulfide bonds in proteins to form the native |
| | | targeting | structures. Expression of ER-60 is upregulated in |
| | | | lactating mammary tissue and in mammary cells in |
| | | | culture in response to prolactin indicating an |
| | | | important role in mammary protein synthesis. |
| 41 | 172 | Metabolism, lipid, | Fatty acid-binding protein, adipocyte, AFABP, also |
| | | intracellular transport | known as adipocyte lipid-binding protein, ALBP |
| | | | plays a role in the intracellular transport of long- |
| | | | chain fatty acids and their acyl-CoA esters making |
| | | | them available for fatty acid oxidation or |
| | | | glycerolipid synthesis. |
| 42 | 173 | Metabolism, lipid, | Fatty acid-binding protein, heart H-FABP, also |
| | | intracellular transport | known as mammary-derived growth inhibitor, |
| | | | MDGI, plays a role in the intracellular transport of |
| | | | long-chain fatty acids and their acyl-CoA esters |
| | | | making them available for fatty acid oxidation or |
| | | | glycerolipid synthesis. MDGI reversibly inhibits |
| | | | proliferation of mammary carcinoma cells. It is |
| | | | expressed in mammary epithelial cells of |
| | | | developing lobuloalveolar structures and heart. |
| 43 | 174 | Metabolism, | Ferritin heavy chain, ferritin H subunit, is an |
| | | transport, mineral | intracellular molecule that stores iron in a soluble, |
| | | | nontoxic, readily available form. The functional |
| | | | molecule, which is composed of 24 chains, is |
| | | | roughly spherical and contains a central cavity in |
| | | | which the polymeric ferric iron core is deposited. |
| | | | There are two types of ferritin subunits: L (light) |
| | | | chain and H (heavy) chain. The major chain can be |
| | | | light or heavy, depending on the species and tissue |
| | | | type. The H-form is expressed in the heart and |
| | | | mammary tissue. |
| 44 | 175 | Gene/protein | Fragile X mental retardation syndrome related |
| | | expression, protein | protein 1 is a RNA-binding protein that interacts |
| | | synthesis, translation | with FMR1 and FXR1. Fragile X mental |
| | | | retardation syndrome is the most common cause of |
| | | | hereditary mental retardation and is directly |
| | | | associated with the lack of expression of the FMR1 |
| | | | gene that encodes an RNA binding protein. FXR1 |
| | | | is highly homologous to FMR1 and encodes a |
| | | | protein which, like FMR1, contains two KH |
| | | | domains and is highly conserved in vertebrates. |
| | | | The 3′ untranslated regions (3′UTRs) of the human |
| | | | and Xenopus laevis FXR1 mRNAs are strikingly |
| | | | conserved (approximately 90% identity), |
| | | | suggesting conservation of an important function. |
| | | | The KH domains of FXR1 and FMR1 are almost |
| | | | identical, and the two proteins have similar RNA |
| | | | binding properties in vitro. FXR1 and FMR1 are |
| | | | expressed in many tissues, and both proteins, which |
| | | | are cytoplasmic, can be expressed in the same cells. |
| | | | Cells from a fragile X patient that do not have any |
| | | | detectable FMR1 express normal levels of FXR1. |
| | | | These findings demonstrate that FMR1 and FXR1 |
| | | | are members of a gene family and suggest a |
| | | | biological role for FXR1 that is related to that of |
| | | | FMR1. |
| 45 | 176 | Cell signaling, | Growth arrest and DNA-damage-inducible protein, |
| | | protein modification, | GADD45 gamma, mediates activation of stress- |
| | | kinase | responsive kinase MTK1 (also known as MEKK4 |
| | | | or MAPKKK) to regulate the p38 and JNK MAPK |
| | | | pathways for the control of cell cycle and |
| | | | apoptosis. MyD118 and GADD45 are two related |
| | | | genes that encode proteins that play important roles |
| | | | such negative growth control. |
| 46 | 177 | Metabolism, sugar, | Galactokinase 2, GALK2 or GK2 catalyzes the first |
| | | other sugars | reaction of galactose metabolism to produce D- |
| | | | galactose 1-phosphate. |
| 47 | 178 | Cell signaling, | Guanine nucleotide-binding protein beta, subunit- |
| | | receptors, membrane | like protein 12.3, also known as P205, receptor of |
| | | | activated protein kinase C 1 or RACK1, binds |
| | | | protein kinase C and acts as an intracellular |
| | | | receptor to anchor the activated PKC to the |
| | | | cytoskeleton and thereby regulating the activity of |
| | | | protein kinase C and protein kinase C-mediated cell |
| | | | signaling. |
| 48; 49 | 179; 180 | Cell/organism | Ig gamma-2 chain constant region. Component of |
| | | defense, | the IgG2 complex of antibodies |
| | | immunology, | |
| | | antibody | |
| 50 | 181 | Metabolism, lipid, | Glycerol-3-phosphate dehydrogenase [NAD+], |
| | | phospholipid | cytoplasmic belongs to the NAD-dependent |
| | | synthesis | glycerol-3-phosphate dehydrogenase family and |
| | | | reversibly converts glycerol-3-phosphate to |
| | | | dihydroxyacetone phosphate. |
| 51 | 182 | Cell signaling, | Growth regulated protein belongs to the intercrine |
| | | extracellular factor, | alpha family (small cytokine C-X-C or chemokine |
| | | cytokine | CXC) and has chemotactic activity for neutrophils. |
| 52 | 183 | Cell/organism | Glutathione S-transferase theta 1, GST class-theta, |
| | | defense, homeostasis, | catalyzes the conjugation of reduced glutathione to |
| | | antioxidant | a wide number of exogenous and endogenous |
| | | | hydrophobic electrophiles. It is found in |
| | | | erythrocyte and expressed at low levels in liver. In |
| | | | lung, it is expressed at low levels in Clara cells and |
| | | | ciliated cells at the alveolar/bronchiolar junction, |
| | | | but absent from epithelial cells of larger |
| | | | bronchioles. |
| 53; 54 | 184; 185 | Cell/organism | Immunoglobulin-related 14.1 protein belongs to the |
| | | defense, | immunoglobulin superfamily and shows similarity |
| | | immunology, | to lambda light chain in C-terminal half (J and C |
| | | antibody | regions). |
| 55 | 186 | Gene/protein | Eukaryotic translation initiation factor 3 subunit 4, |
| | | expression, protein | EIF-3 delta, is part of the EIF-3 complex that is |
| | | synthesis, translation | composed of at least 10 different subunits and |
| | | | binds to the 40S ribosome and promotes the |
| | | | binding of methionyl-tRNAi and mRNA. This |
| | | | subunit binds to the 18S rRNA. |
| 56 | 187 | Cell/organism | Kappa light chain is a component of the IgA and |
| | | defense, | IgG antibodies. |
| | | immunology, | |
| | | antibody | |
| 57 | 188 | Cell/organism | Ig lambda chain is a component of the IgA and IgG |
| | | defense, | classes of antibodies. |
| | | immunology, | |
| | | antibody | |
| 58 | 189 | Metabolism, sugar, | L-lactate dehydrogenase H chain, LDH-B, |
| | | glycolysis | catalyzes the final step in anaerobic glycolysis. |
| | | | There are three types of LDH chains: M (LDH-A) |
| | | | found predominantly in muscle tissues, H (LDH-B) |
| | | | found in heart muscle and X (LDH-C) which is |
| | | | present in the spermatozoa of mammals, in the |
| | | | Columbidae birds and in Actinopterygian fish |
| 59 | 190 | Metabolism, lipid, | Lipoprotein lipase hydrolyses triglycerides of |
| | | lipase | circulating chylomicrons and very low density |
| | | | lipoproteins (VLDL). The enzyme functions in the |
| | | | presence of apolipoprotein C-2 on the luminal |
| | | | surface of vascular endothelium and is attached to |
| | | | the membrane by a GPI-anchor. Defects in LPL |
| | | | are a cause of chylomicronemia syndrome (also |
| | | | known as type I hyperlipoproteinemia). |
| 60 | 191 | Cell signaling, | Lipophilin B precursor binds androgens and other |
| | | extracellular factor, | steroids. It also binds estramustine, a |
| | | binding protein | chemotherapeutic agent used for prostate cancer. |
| | | | Lipophilin components A, B and C are human |
| | | | homologues of prostatein, the major secreted |
| | | | protein of rat prostate. The lipophilin B gene |
| | | | resides on chromosome 10q23, a region deleted in |
| | | | many tumors. Lipophilin gene products are widely |
| | | | expressed in normal tissues, especially in |
| | | | endocrine-responsive organs. Highest expression is |
| | | | found in skeletal muscle, but also in thymus, |
| | | | trachea, kidney, steroid responsive tissues |
| | | | (prostate, testis, uterus, breast and ovary), and |
| | | | salivary gland. Lipopholin B belongs to the |
| | | | uteroglobin family in the lipophilin subfamily. |
| 61 | 192 | Cell/organism | Ig lambda chain V-II region is the light chain of the |
| | | defense, | immunoglobulin alpha class of antibodies, IgA. |
| | | immunology, | IgA is the major class of secreted antibodies and is |
| | | antibody | responsible for providing immunity to mucosal |
| | | | surfaces. |
| 62 | 193 | Cell/organism | MUTL protein homolog 1 also known as DNA |
| | | defense, homeostasis, | mismatch repair protein MLH1, is involved in the |
| | | DNA repair | repair of mismatches in DNA. The active protein |
| | | | is a heterodimer of PMS2 and MLH1. MHL1 is |
| | | | expressed in the colon, lymphocytes, breast, lung, |
| | | | spleen, testis, prostate, thyroid, gall bladder and |
| | | | heart. MHL1 is also associated with familial |
| | | | hereditary non-polyposis colon cancer (HNPCC) |
| | | | (Lynch syndrome). HNPCC is one of the most |
| | | | common genetic diseases in the Western world, |
| | | | and accounts for 15% of all colon cancers. It is |
| | | | often divided into two subgroups. Type I: |
| | | | hereditary predisposition to colorectal cancer, a |
| | | | young age of onset, and carcinoma observed in the |
| | | | proximal colon. Type II: patients have an increased |
| | | | risk for cancers in certain tissues such as the uterus, |
| | | | ovary, breast, stomach, small intestine, skin, and |
| | | | larynx in addition to the colon. |
| 63 | 194 | Metabolism, | Golgi 4-transmembrane spanning transporter MTP |
| | | transport, | is an integral membrane protein that functions in |
| | | intracellular | the transport of nucleosides and/or nucleoside |
| | | | derivatives between the cytosol and the lumen of |
| | | | an intracellular membrane-bound compartment. |
| | | | The C-terminal domain is necessary for retention |
| | | | within intracellular membranes. |
| 64 | 195 | Cell/organism | Ig mu chain C region is a part of the |
| | | defense, | immunoglobulin M (IgM) complex that is the first |
| | | immunology, | antibody molecule produced in the immune |
| | | antibody | response. During differentiation, B lymphocytes |
| | | | switch from expression of membrane-bound IgM to |
| | | | secretion of IgM. The mu chains of membrane and |
| | | | secreted IgM differ in their C- terminal segments. |
| 65 | 196 | Metabolism, sugar, | N-acetyllactosamine synthase is also known as N- |
| | | other sugars | acetylglucosamine (beta 1- |
| | | | >4)galactosyltransferase, lactose synthase A |
| | | | protein or galactosyltransferase (GT). N- |
| | | | acetyllactosamine synthase is responsible for the |
| | | | synthesis of complex-type N-linked |
| | | | oligosaccharides in many glycoproteins a as well as |
| | | | the carbohydrate moieties of glycolipids. It is a |
| | | | type II membrane protein. N-acetyllactosamine |
| | | | synthase exists in two forms: a membrane-bound |
| | | | form in trans cisternae of the Golgi apparatus and a |
| | | | soluble form in body fluids. The soluble form |
| | | | derives from the membrane form by proteolytic |
| | | | processing. Two isoforms have been identified: a |
| | | | long form and a short form that are produced by |
| | | | alternative splicing. |
| 66 | 197 | Gene/protein | Nucleophosmin, NPM, also known as nucleolar |
| | | expression, RNA | phosphoprotein B23, numatrin, nucleolar protein |
| | | synthesis, RNA | no38, is associated with nucleolar |
| | | processing | ribonucleoprotein structures and binds single- |
| | | | stranded nucleic acids. It functions in the assembly |
| | | | and/or transport of ribosomes. It is generally |
| | | | nucleolar, but is translocated to the nucleoplasm in |
| | | | case of serum starvation or treatment with |
| | | | anticancer drugs. |
| 67 | 198 | | Human L1 element L1.6 or putative p150 gene, |
| | | | contains a major class of mobile elements that is |
| | | | expanding in the mouse genome. |
| | | | Retrotransposition of LINEs and other |
| | | | retroelements increases repetition in mammalian |
| | | | genomes and can cause deleterious mutations. |
| | | | Active LINE-1 (L1) elements possess the ability to |
| | | | transduce non-L1 DNA flanking their 3′ ends to |
| | | | new genomic locations. The progenitor L1 |
| | | | elements encode a site-specific endonuclease and |
| | | | generate copies that are inserted at these specific |
| | | | sites. |
| 68 | 199 | Gene/protein | STAT induced STAT inhibitor-2, SSI-2, also |
| | | expression, RNA | known as suppresser of cytokine signaling, SOCS- |
| | | synthesis, | 2, is induced in response to cytoline signaling and |
| | | transcription factors | plays a critical role in negative feedback control of |
| | | | JAK-STAT signaling pathway. |
| 69 | 200 | Gene/protein | Elongation factor 1 alpha is a member of a |
| | | expression, protein | polymorphic multi-gene family of proteins required |
| | | synthesis, translation | for protein synthesis. |
| 70 | 201 | Metabolism, | Cationic amino acid transporter 3, CAT3, mediates |
| | | transport, cellular | the transmembrane uptake of cationic amino acids |
| | | uptake | such as lysine and arginine. |
| 71 | 202 | Cell/organism | DNA recombination and repair protein HNGS1, or |
| | | defense, homeostasis, | MRE11A, along with protein RAD50 are known to |
| | | DNA repair | be required for nonhomologous joining of DNA |
| | | | ends in vivo. MRE11 by itself has 3′ to 5′ |
| | | | exonuclease activity that is increased when Mre11 |
| | | | is in a complex with RAD50. MRE11 also exhibits |
| | | | endonuclease activity, as shown by the asymmetric |
| | | | opening of DNA hairpin loops. In conjunction with |
| | | | a DNA ligase, MRE11 promotes the joining of |
| | | | noncomplementary ends in vitro by utilizing short |
| | | | homologies near the ends of the DNA fragments. |
| 72 | 203 | Cell signaling, | Sorting nexin-1 (SNX1) is responsible for the |
| | | receptors, membrane | ligand-induced internalization of the epidermal |
| | | | growth factor receptor (EGFR) leading to |
| | | | accelerated receptor degradation. It binds to a |
| | | | region, of the epidermal growth factor receptor |
| | | | (EGFR) containing the lysosomal, targeting code |
| | | | and plays a role in sorting EGFR to lysosomes. |
| 73 | 204 | Gene/protein | Sperm specific protein is encoded by a testis |
| | | expression, RNA | mRNA specifically expressed in testicular haploid |
| | | synthesis, | germ cells, having unique palindromic sequences |
| | | transcription factors | and encoding a leucine zipper dimerization motif |
| 74 | 205 | Cell/organism | HSP89-alpha-delta-N is a member of the HSP90 |
| | | defense, homeostasis, | gene family that encodes abundant molecular |
| | | stress response | chaperones in the eukaryotic cytosol, that are |
| | | | involved in the folding of a set of cell regulatory |
| | | | proteins and in the re-folding of stress-denatured |
| | | | polypeptides. |
| 75 | 206 | Gene/protein | Zinc finger protein 216 is a member of a family of |
| | | expression, RNA | putative transcription factors that are characterized |
| | | synthesis, | by their ability to form zinc fingers that can bind |
| | | transcription factors | DNA. |
| 76 | 207 | Gene/protein | Translation initiation factor eIF2C is a 94 kDa |
| | | expression, protein | protein that has been shown to play important roles |
| | | synthesis, translation | in the eukaryotic peptide chain initiation process. |
| 77 | 208 | Metabolism, sugar, | Blood group A transferase is a UDP-GalNAc: Fuc |
| | | other sugars | alpha 1---2Gal alpha 1---3GalNAc transferase |
| | | | (histo-blood group A transferase). |
| 78 | 209 | Cell signaling; | MEK binding partner 1, is a MEK binding partner |
| | | protein modification, | that enhances enzymatic activation of the MAP |
| | | kinase | kinase cascade. |
| 79 | 210 | Gene/protein | Bromodomain-containing protein BP75. A |
| | | expression, RNA | bromodomain is a motif characteristic of certain |
| | | synthesis, | transcriptional coactivators and histone |
| | | transcription factors | acetyltransferases |
| 80 | 211 | Gene/protein | Snurportin1. The nuclear import of the |
| | | expression, RNA | spliceosomal snRNPs U1, U2, U4 and U5, is |
| | | synthesis, RNA | dependent on the presence of a complex nuclear |
| | | processing | localization signal (NLS). The latter is composed |
| | | | of the 5′-2, 2, 7-terminal trimethylguanosine (m3G) |
| | | | cap structure of the U snRNA and the Sm core. |
| | | | domain. Snurportin1 interacts specifically with |
| | | | m3G-cap but not m7G-cap structures. Snurportin1 |
| | | | enhances the m3G-cap dependent nuclear import of |
| | | | U snRNPs in both Xenopus laevis oocytes and |
| | | | digitonin-permeabilized HeLa cells, demonstrating |
| | | | that it functions as a snRNP-specific nuclear import |
| | | | receptor. Snurportin1 represents a novel nuclear |
| | | | import receptor which contains an N-terminal |
| | | | importin beta binding (IBB) domain, essential for |
| | | | function, and a C-terminal m3G-cap-binding region |
| | | | with no structural similarity to the ARM repeat |
| | | | domain of importin alpha. |
| 81 | 212 | | 31.7 kDa protein. |
| 82 | 213 | | Protein encoding a novel acyl transferase. |
| 83 | 214 | Cell signaling, | Fe65L2 protein interacts with the intracellular |
| | | receptors, membrane | domain of the Alzheimer's beta-amyloid precursor |
| | | | protein (APP) and APP-like proteins through its |
| | | | carboxyl terminal domain. Its mRNA is expressed |
| | | | in various tissues; a transcript of about 2.2 kb is |
| | | | found in brain. A splicing variant lacking two |
| | | | amino acids in the first PID/PTB element was |
| | | | detected. |
| 84 | 215 | Cell signaling, | CD81 antigen, 26 kDa cell surface protein TAPA- |
| | | receptors, membrane | 1, is an integral membrane protein that plays an |
| | | | important role in the regulation of lymphoma |
| | | | growth. It interacts with a 16-kDa Leu-13 protein |
| | | | to form a complex that is involved in signal |
| | | | transduction. It is expressed in hematolymphoid, |
| | | | neuroectodermal, and mesenchymal tumor cell |
| | | | lines. |
| 85 | 216 | | Protein encoding a reverse transcriptase-like |
| | | | protein. |
| 86 | 217 | Metabolism, amino | Ornithine decarboxylase antizyme binds to, and |
| | | acid, degradation | destabilizes, ornithine decarboxylase, which is then |
| | | | degraded: A ribosomal frameshift occurs between |
| | | | codons for SER-57 and ASP-58. An autoregulatory |
| | | | mechanism enables modulation of frameshifting |
| | | | according to the cellular concentration of |
| | | | polyamines. |
| 87 | 218 | Metabolism, lipid, | Mitochondrial import receptor subunit TOM20 |
| | | intracellular transport | homolog, is the central component of the receptor |
| | | | complex responsible for the recognition and |
| | | | translocation of cytosolically synthesized |
| | | | mitochondrial preproteins. Together with TOM22, |
| | | | it functions as the transit peptide receptor at the |
| | | | surface of the mitochondrion outer membrane and |
| | | | facilitates the movement of preproteins into the |
| | | | translocation pore. |
| 88 | 219 | Gene/protein | Polypeptide N-acetylgalactosaminyltransferase, (ec |
| | | expression, protein | 2.4.1.41) protein- UDP acetylgalactosaminyltransferase, |
| | | synthesis, post- | UDP-GALNAC: polypep-tide, |
| | | translational | N-acetylgalactosaminyltransferase, ALNAC-T1, is |
| | | modification/targeting | a type II membrane protein located in the Golgi |
| | | | that catalyzes the initial reaction in O-linked |
| | | | oligosacceharide biosynthesis, the transfer of an N- |
| | | | acetyl-D-galactosamine residue to a serine or |
| | | | threonine residue on the protein receptor. |
| 89 | 220 | Gene/protein | Peroxisomal targeting signal 2 receptor, PTS2 |
| | | expression, protein | receptor, peroxin-7, binds to the N-terminal PTS2- |
| | | synthesis, post- | type peroxisomal targeting signal and, though the |
| | | translational | interaction with PEX5, plays an essential role in |
| | | modification/ | peroxisomal protein import. |
| | | targeting | |
| 90 | 221 | Metabolism, lipid, | Glycerol-3-phosphate acyltransferase, |
| | | phospholipid | mitochondrial, GPAT, is an integral membrane |
| | | synthesis | protein that catalyzes the first step in de novo |
| | | | phospholipid biosynthesis and also functions in the |
| | | | regulation of membrane biogenesis. |
| 91 | 222 | Metabolism, | Purine nucleoside phosphorylase, PNP, EC 2.4.2.1 |
| | | cofactor, synthesis | or inosine phosphorylase cleaves purine |
| | | | nucleosides to produce the purine plus ribose 1- |
| | | | phosphate and plays a critical role in determining |
| | | | the intracellular concentration of purine |
| | | | nucleosides. |
| 92 | 223 | Cell | Undulin 1 (matrix glycoprotein), is a member of |
| | | structure/motility, | the fibronectin-tenascin family of noncollagenous |
| | | extracellular matrix | extracellular matrix glycoproteins. Undulin |
| | | | associates with collagen fibrils and serves a |
| | | | specific function in the supramolecular |
| | | | organization of collagen fibrils in soft tissues. |
| 93 | 224 | Cell signaling, | SLP-76 tyrosine phosphoprotein is a 76-kDa |
| | | protein modification, | tyrosine phosphoprotein associated with the |
| | | kinase | adaptor protein Grb2 in T cells and plays an |
| | | | important role in TCR-mediated intracellular signal |
| | | | transduction. |
| 94 | 225 | Gene/protein | Alpha-2-macroglobulin is a serum anti-proteinase |
| | | expression, protein | of the serpin family that is induced in response to |
| | | degradation, protease | inflammation. |
| | | inhibitor | |
| 95 | 226 | Gene/protein | Nuclear receptor coactivator 4, also known as 70 kDa |
| | | expression, RNA | androgen receptor coactivator, 70 kDa AR- |
| | | synthesis, | activator, RET-activating protein. ELE1, enhances |
| | | transcription factors | the androgen receptor transcriptional activity in |
| | | | target cells and is responsible for the ligand- |
| | | | independent coactivator of the peroxisome |
| | | | proliferator-activated receptor (PPAR) gamma. It |
| | | | interacts with the androgen receptor and the |
| | | | retinoid X receptor (RXR) in a ligand-dependent |
| | | | manner. At least 2 isoforms, alpha and beta, may |
| | | | be produced by alternative splicing. It is widely |
| | | | expressed. |
| 96 | 227 | Cell signaling, | Type II receptor for bone morphogenetic, protein-4 |
| | | receptors, membrane | (BMPR-II). Bone morphogenetic proteins (BMPs) |
| | | | are members of the transforming growth factor beta |
| | | | superfamily. Several members of this family have |
| | | | been shown to transduce their signals through |
| | | | binding to type I and type II serine-(threonine) |
| | | | kinase receptors. BMPR-II is a mammalian type II |
| | | | receptor for BMPs binds osteogenic protein (OP)- |
| | | | 1/BMP-7 and less efficiently BMP-4 only weakly |
| | | | alone, but the binding was facilitated by the |
| | | | presence of other type I receptors for BMPs |
| | | | indicating a requirement for the formation of |
| | | | heteromeric complexes with bone morphogenetic |
| | | | protein. |
| 97 | 228 | Metabolism, lipid, | Fatty acid synthase (EC 2.3.1.85) is a multi-domain |
| | | fatty acid synthesis | protein that catalyzes the synthesis of long chain |
| | | | fatty acids from acetyl-CoA and malonyl-CoA. |
| 98 | 229 | Cell | Intestinal trefoil factor is secreted by goblet cells of |
| | | structure/motility, | small and large intestinal epithelia and also by the |
| | | extracellular matrix | uterus and mammary gland and has a role in |
| | | | promoting cell migration. |
| 99 | 230 | Cell signaling, | TAK1 (TGF-beta-activated kinase) is a member of |
| | | protein modification, | the MAPKKK family as a, potential mediator of |
| | | kinase | TGF-beta signal transduction mitogen-activated |
| | | | protein kinase (MAPK) pathway. This pathway is a |
| | | | conserved, eukaryotic signaling module that |
| | | | converts receptor signals into various outputs. |
| | | | MAPK is activated through phosphorylation by |
| | | | MAPK kinase, (MAPKK), which is first activated |
| | | | by MAPKK kinase (MAPKKK). Furthermore, the |
| | | | kinase activity of TAK1 was stimulated in response |
| | | | to TGF-beta and bone morphogenetic protein |
| | | | indicating that TAK1 functions as a mediator in the |
| | | | signaling pathway of TGF-beta superfamily |
| | | | members. |
| 100 | 231 | Cell signaling, | Rad GTPase is a member of the Ras-guanosine |
| | | receptors, G-protein- | triphosphatase superfamily and was termed Rad |
| | | associated | (Ras associated with diabetes) because it is |
| | | | overexpressed in the muscle of individuals with |
| | | | Type II diabetics as compared to normal |
| | | | individuals. Messenger ribonucleic acid of Rad |
| | | | was expressed primarily in skeletal and cardiac |
| | | | muscle and was increased several-fold in the |
| | | | muscle of Type II diabetics as compared to normal |
| | | | individuals. It has also been detected in lesser |
| | | | amounts in lung placenta and kidney and in adipose |
| | | | tissue. |
| 101 | 232 | Gene/protein | Translocational protein-1, HTP-1 or SEC62, is a |
| | | expression, protein | mammalian homolog of a yeast protein that is |
| | | synthesis, post- | involved in protein translocation across and into the |
| | | translational | endoplasmic reticulum (ER) membrane. Two |
| | | modification/ | HTP1 transcripts of about 2.8 and 5.5 kb, are |
| | | targeting | expressed concomitantly in various human tissues |
| | | | such as heart, brain, placenta, liver and pancreas. |
| | | | This protein is likely to be important in the |
| | | | secretion of milk proteins. |
| 102 | 233 | Cell division, | Fatso (Fto) is expressed throughout embryonic |
| | | apoptosis | development and is also found in tissues of adult |
| | | | mice. The expression data of Fto define it as a |
| | | | candidate gene involved in processes such as |
| | | | programmed cell death, craniofacial development, |
| | | | and establishment of left-right asymmetry. |
| 103 | 234 | Cell/organism | Bovine leukocyte antigen is similar to the MHC |
| | | defense, | class II DBQ genes. |
| | | immunology, MHC | |
| 104 | 235 | Cell signaling, | Tousled-like kinase 2, TLK2, is a mammalian |
| | | protein modification, | homolog of the plant gene Tousled (TSL) that |
| | | kinase | encodes a serine/threonine kinase, essential for |
| | | | proper flower development. The mammalian TLKs |
| | | | share several functional properties with plant TSL, |
| | | | including a broad expression, a propensity to |
| | | | dimerize and autophosphorylate, and a preference |
| | | | for similar substrates. In particular, TLKs are cell- |
| | | | cycle-regulated enzymes, displaying maximal |
| | | | activities during S phase, TLK2 is regulated by |
| | | | cell-cycle-dependent phosphorylation. Drug- |
| | | | induced inhibition of DNA replication causes a |
| | | | rapid loss of TLK activity, indicating that TLK |
| | | | function is tightly linked to ongoing DNA |
| | | | replication. |
| 106 | 237 | Gene/protein | C2H2 zinc finger protein PLAGL1 and the two |
| | | expression, RNA | new PLAG1 family members constitute a novel |
| | | synthesis, | subfamily of zinc finger proteins that recognize |
| | | transcription factors | DNA and/or RNA and control the expression of |
| | | | specific genes. PLAGL1 exhibits antiproliferative |
| | | | activities and is a tumor suppressor gene candidate. |
| 107 | 238 | | Homolog of Drosophila melanogaster (fruit fly) |
| | | | cg15084 protein. |
| 109 | 240 | | Homolog of the Drosophila melanogaster (fruit |
| | | | fly) cg7085 protein. |
| 110 | 241 | | Protein similar to the ATP-dependent |
| | | | metalloprotease FTSH1. |
| 111 | 242 | Gene/protein | Prediabetic NOD sera-reactive autoantigen, is an |
| | | expression; protein | autoantigen related to the pathogenic mechanism of |
| | | degradation, | insulin dependent diabetes mellitus and that also |
| | | ubiquitin | contains a region that is similar to a sequence in |
| | | | cullins, a family of proteins implicated in the |
| | | | ubiquitination of G1 phase cyclins and cyclin- |
| | | | dependent kinase inhibitors. The cullins may be |
| | | | related members of a ubiquitin ligase family that |
| | | | targets the degradation cell cycle regulators. |
| 112 | 243 | Cell signaling, | BRI gene encodes a transmembrane protein. A |
| | | receptors, membrane | stop-codon mutation in the BRI gene associated |
| | | | with familial British dementia. |
| 113 | 244 | Cell division, DNA | Sarcolectin (SCL) is a 55 kDa protein that |
| | | synthesis/replication, | stimulates DNA synthesis in all immunocompetent |
| | | polymerase | cells and inhibits the synthesis and the expression |
| | | | of the IFN dependent secondary proteins and thus |
| | | | the interferon (IFN)-dependent antiviral state. |
| | | | Interferons (IFNs) are major cytokines, responsible |
| | | | for down-regulating cell growth and for promoting |
| | | | cell differentiation. SCL blocks in the cells the |
| | | | established IFN-dependent interphase and |
| | | | stimulates DNA synthesis, probably in co- |
| | | | ordination with more specific growth factors or |
| | | | hormones. IFN and SCL proteins are therefore part |
| | | | of a feedback system operating the regulation of |
| | | | normal growth. In pathological cases, SCL could |
| | | | play a role in the development of tumors in juvenile |
| | | | osteosarcomas or in AIDS. A great variety of |
| | | | vertebrate cells contain detectable amounts of |
| | | | lectins, able to stimulate the initiation of cellular |
| | | | DNA synthesis. |
| 114 | 245 | | 21.9 kDa protein., |
| 115 | 246 | Gene/protein | Zinc finger protein is a member of a class of DNA- |
| | | expression, RNA | binding proteins, which act as transcription factors |
| | | synthesis, | binding specifically to short DNA-sequences and |
| | | transcription factors | controlling the transcription of a number of genes. |
| 116 | 247 | Gene/protein | Brefeldin A-inhibited guanine nucleotide-exchange |
| | | expression, protein | protein 2 is involved in the activation of ADP- |
| | | synthesis, post- | ribosylation factors (ARFs) which are 20-kDa |
| | | translational | guanine nucleotide-binding proteins that play an |
| | | modification/ | important role in intracellular vesicular, trafficking. |
| | | targeting | Two major families of ARF guanine nucleotide- |
| | | | exchange proteins (GEPs) are known, one of |
| | | | which consists of approximately 200-kDa |
| | | | molecules that are inhibited by brefeldin A (BFA). |
| | | | BFA is a fungal metabolite that blocks protein |
| | | | secretion and causes apparent disintegration of |
| | | | Golgi structure, and approximately 50-kDa GEPs |
| | | | that are insensitive to BFA. ARFs are active and |
| | | | associate with membranes when GTP is bound, |
| | | | whereas inactive ARF·GDP is cytosolic. |
| | | | Replacement of GDP by GTP is accelerated by |
| | | | ARF GEPs. This protein is therefore important in |
| | | | the regulation of secretion of proteins from the |
| | | | mammary gland. |
| 117 | 248 | Cell/organism | NAD+ ADP-ribosyltransferase 2, ADPRT2, is a |
| | | defense, homeostasis, | member of a family of poly(ADP-ribose) |
| | | DNA repair | polymerases (PARPs) that is involved in the |
| | | | posttranscriptional modification of nuclear proteins |
| | | | by poly(ADP-ribosyl)ation in response to DNA |
| | | | strand breaks and plays an important role in DNA |
| | | | repair, regulation of apoptosis, and maintenance of |
| | | | genomic stability. |
| 118 | 249 | | Novel protein with GTP-binding domain. |
| 119 | 250 | Gene/protein | Peptide elongation factor 1-beta, EF-1-beta, is |
| | | expression, protein | required for the elongation phase of protein |
| | | synthesis, translation | synthesis and stimulates the exchange of GDP |
| | | | bound to EF-1-alpha to GTP. EF-1 is composed of |
| | | | four subunits: alpha, beta, delta, and gamma. |
| | | | Phosphorylation affects the GDP/GTP exchange |
| | | | rate. |
| 120 | 251 | Cell signaling, | Rad GTPase is a member of the Ras-guanosine |
| | | receptors, G-protein- | triphosphatase superfamily and was termed Rad |
| | | associated | (Ras associated with diabetes) because it is |
| | | | overexpressed in the muscle of individuals with |
| | | | Type II diabetics as compared to normal |
| | | | individuals. Messenger ribonucleic acid of Rad |
| | | | was expressed primarily in skeletal and cardiac |
| | | | muscle and was increased several-fold in the |
| | | | muscle of Type II diabetics as compared to normal |
| | | | individuals. It has also been detected in lesser |
| | | | amounts in lung placenta and kidney and in adipose |
| | | | tissue. |
| 121 | 252 | Gene/protein | Heterogeneous nuclear ribonucleoprotein U, |
| | | expression, RNA | hnRNP U, also known as scaffold attachment |
| | | synthesis, RNA | factor A SAF-A, binds to pre-mRNA and has high |
| | | processing | affinity for scaffold-attached region (SAR) DNA. It |
| | | | can bind to double-and single-stranded DNA and |
| | | | RNA and is component of ribonucleosomes where |
| | | | it plays a role in the processing of mRNA. Two |
| | | | forms, a long form and a short form; are produced |
| | | | by alternative splicing and the protein is |
| | | | extensively phosphorylated. |
| 122 | 253 | Cell signaling, | RAS-related protein R-RAS2 also known as RAS- |
| | | receptors, G-protein- | like protein TC21, or teratocarcinoma oncogene, is |
| | | associated | a highly conserved plasma membrane-associated |
| | | | GTP-binding protein with GTPase activity. TC21 |
| | | | transduces growth inhibitory signals across the cell |
| | | | membrane, exerting its effect through an effector |
| | | | shared with the RAS proteins but in an antagonistic |
| | | | fashion. It is ubiquitously present in all tissues |
| | | | examined, with the highest levels in heart, placenta, |
| | | | and skeletal muscle. Moderate levels in lung and |
| | | | liver; low levels in brain, kidney, and pancreas. |
| | | | Defects in TC21 can be the cause of ovarian |
| | | | tumors. |
| 123 | 254 | Gene/protein | Small nuclear ribonucleoprotein G, snrNP-G, SM |
| | | expression, RNA | protein G, is a nuclear protein of the spliceosomal |
| | | synthesis, RNA | small nuclear ribonucleoproteins snrNP SM |
| | | processing | proteins family and associates with sn-RNP U1, U2 |
| | | | U4/U6 and U5 and plays an essential role in the |
| | | | biogenesis of the snRNPs and in RNA processing. |
| 124 | 255 | Gene/protein | 15 kDa Selenoprotein is one of 11 known |
| | | expression, secreted | mammalian proteins that contains selenocysteine. |
| | | protein | Selenium occurs in proteins as the amino acid, |
| | | | selenocysteine and is essential for numerous |
| | | | biological processes. The 15-kDa protein is |
| | | | expressed in a wide range of tissues, with increased |
| | | | levels in the thyroid, parathyroid, and prostate- |
| | | | derived cells. |
| 125 | 256 | Cell | Transgelin also known as smooth muscle protein |
| | | structure/motility, | 22-alpha, SM22-alpha, WS3-10, 22 kDa actin- |
| | | cytoskeletal | binding protein, is a protein involved in the cross- |
| | | | linking and gelling of actin. It is involved in |
| | | | calcium interactions and contractile properties of |
| | | | cells that may contribute to replicative senescence. |
| 126 | 257 | Gene/protein | Translocon-associated protein, delta subunit, |
| | | expression, protein | TRAP-delta also known as signal sequence |
| | | synthesis, post- | receptor delta subunit, SSR-delta is a type I |
| | | translational | membrane protein found in endoplasmic reticulum |
| | | modification/ | and part of a complex whose function is to bind |
| | | targeting | Ca(2+) to the endoplasmic reticulum membrane |
| | | | and thereby regulate the retention of endoplasmic |
| | | | reticulum resident proteins. The active protein is a |
| | | | heterotetramer of TRAP-alpha, TRAP-beta; TRAP- |
| | | | delta and TRAP-gamma. |
| 127 | 258 | Cell/organism | Microsomal stress 70 protein belongs to the heat |
| | | defense, homeostasis, | shock protein 70 family and has peptide- |
| | | stress response | independent ATPase activity. It plays a central role |
| | | | in the processing of cytosolic and secretory |
| | | | proteins. |
| 128 | 259 | Cell/organism | Phenol-sulfating phenol sulfotransferase, P-PST, |
| | | defense, homeostasis, | belongs to the sulfotransferases family and |
| | | stress response | catalyzes the sulfate conjugation of catecholamines |
| | | | and of phenolic drugs. |
| 129 | 260 | Gene/protein | Transcription initiation factor TFIID 31 kDa |
| | | expression, RNA | subunit, TAFII-31, is a component of the |
| | | synthesis, | transcription factor IID (TFIID) complex that is |
| | | transcription factors | essential for mediating regulation of RNA |
| | | | polymerase. TAFII31 is a coactivator for the P53 |
| | | | protein and. Also interacts with the acidic |
| | | | transactivator viral protein 16 (VP16) as well as |
| | | | with the general transcription factor TFIIB. |
| 130 | 261 | Gene/protein | T-complex protein 1, zeta subunit, also known as |
| | | expression, protein | TCP-1-zeta, CCT-zeta or CCT-zeta-1, is a |
| | | synthesis, post- | molecular chaperone that assists the folding of |
| | | translational | proteins upon ATP hydrolysis. It is known to play a |
| | | modification/ | role in vitro in the folding of actin and tubulin. It |
| | | targeting | is a hetero-oligomeric complex of about 850 to 900 kDa |
| | | | that forms two stacked rings, 12 to 16 nm in |
| | | | diameter. It is located in the cytoplasm and |
| | | | expressed in all tissues examined. |
| 131 | 262 | Gene/protein | Thyroid receptor interacting protein 15, TRIP15, is |
| | | expression, RNA | a member of a family of thyroid receptor |
| | | synthesis, | interacting proteins (TRIPS) that specifically |
| | | transcription factors | interact with the ligand binding domain of the |
| | | | thyroid receptor (TR). TRIP15 does not require the |
| | | | presence of thyroid hormone for its interaction. |

The polynucleotides of SEQ ID NO 1, 14, 22, 23, 31, 32, 51, 60, 84, 96, 120, 122 and 124 encode polypeptides involved in cell signalling at the extracellular level, including both secreted polypeptides and cell surface receptors for secreted polypeptides. These function in regulating cell metabolism and cell growth. The polynucleotides of SEQ ID NO: 102 and 113 encode polypeptides that are involved in cellular differentiation. The polynucleotides of SEQ ID NO 8, 10, 11, 45, 47, 72, 78, 83, 93, 100, 104 and 112 encode polypeptides that are intracellular mediators of external cell signalling events. The polynucleotides of SEQ ID NO 12, 17, 24, 27, 37, 92, 98, 108 and 125 encode polypeptides that are part of the cellular cytoskeleton and extracellular matrix, and that have utility in the manipulation of mammary epithelial cell structure and function. The polynucleotides of SEQ ID NO: 13, 48, 49, 53, 54, 56, 57, 61, 64 and 103 encode components of the immune system and have utility in enhancing the concentration of immune proteins in mammary secretions. The polynucleotides of SEQ ID NO: 3, 15, 16, 18-20, 30, 36, 41-43, 46, 50, 52, 58, 59, 62, 63, 65, 70, 71, 74, 77, 86, 87, 90, 91, 97, 117, 127 and 128 encode polypeptides involved in intracellular metabolic pathways relating to the synthesis and degradation of lipids, carbohydrates and purines, and the oxidation of xenobiotics. The polynucleotides of SEQ ID NO: 2, 4, 5, 7, 9, 21, 25, 26, 28, 29, 33-35, 38, 39, 40, 44, 55, 66, 68, 69, 73, 75, 76, 79, 80 88, 89, 94, 95, 99, 101, 105, 106, 111, 115, 116, 119, 121, 123, 126 and 129-131 encode polypeptides that are involved in protein synthesis and degradation. They include transcription factors that regulate mRNA synthesis, and polypeptides involved in the transcription process, the processing of mRNA, the translation of mRNA to produce polypeptides and processing and turnover of specific proteins. These polynucleotides have utility in the manipulation of the synthesis of mammary secretions to modify the yields of milk and specific milk proteins.

Yet more specific, credible and substantial utilities for the polynucleotides and polypeptides of the present invention are set out in Table 2, below.

TABLE 2

| | SEQ ID | |
|---|---|---|
| SEQ ID | NO: | |
| NO: | Amino | |
| DNA | acid | UTILITY |
| 1 | 132 | Tissue regeneration and wound healing compositions for in vivo and |
| | | in vitro uses; in vitro screens for modulators of connective tissue |
| | | growth; large-scale recombinant production of connective tissue |
| | | growth factor; production of antibodies for use in detecting and/or |
| | | modulating connective tissue growth factor-mediated processes. |
| 2 | 133 | Modulation of snRNP and RNA processing. |
| 3 | 134 | Screen for modulators of nitric oxide synthesis, catecholamine |
| | | synthesis and sympathetic NS functions; diagnostic for mutations or |
| | | deficiencies in GTP cyclohydrolase I (e.g., hereditary progressive |
| | | dystonia, hyperphenylalaninemia) |
| 4 | 135 | Diagnostic for diseases of cell proliferation involving activation of |
| 5 | 136 | ETS2 expression; screen for mitogens; screen for modulators of |
| | | ETS2 transcriptional regulation of cellular genes involved in cell |
| | | proliferation (e.g., c-fos, jun-B, c-myc); suppression of |
| | | tumorigenicity in cells not expressing endogenous gene. |
| 7 | 138 | Screen for modulators of plasma membrane-and fusion protein- |
| | | vesicle interactions in normal and abnormal cellular processes; |
| | | diagnostic for diseases and conditions involving vesicular |
| | | trafficking; constructing cell and animal models for vesicular |
| | | trafficking diseases and disorders; |
| 8 | 139 | Construction of in vitro cell models for Alzheimer's disease (see, |
| | | e.g., U.S. Pat. No. 5,994,084); screen for inhibitors of signal |
| | | transduction pathways involved in proliferation, cell cycle control, |
| | | differentiation, and autoimmune diseases such as rheumatoid |
| | | arthritis (see, e.g., U.S. Pat. No. 6,098,631) |
| 9 | 140 | In vitro screen for agents that modulate growth and differentiation of |
| | | adenocarcinomas; nucleic acid probes and antibodies for tissue |
| | | localization and expression profiling. |
| 10, 11 | 141, 142 | Iin vitro assay to quantitate HSP level; in vitro screen for modulators |
| | | of HSP induction; cytoprotection of ex vivo and in vivo cells; in vivo |
| | | modulation of heat shock response in stressed, traumatized and |
| | | ageing tissues; preparation of HSP-peptide complexes for use in |
| | | vaccines against specific cancers and infectious diseases. |
| 12 | 143 | In vitro screen for modulators of motility, endocytosis, actin- |
| | | polymerization and novel actin-binding components. |
| 13 | 144 | Modulation of local immunity involving IgA expression in |
| | | epithelioid tissues (e.g., mammary gland, gut); diagnostic for |
| | | disorders associated with defective IgA responses. |
| 14 | 145 | Therapy of vascularization disorders; in vitro blood vessel |
| | | formation; in vitro screen for anti-angiogenic or angiogenic agents; |
| | | delivery of imaging or therapeutic agents to endothelial cell nucleus. |
| 15 | 146 | Screen for modulators of receptor-mediated endocytosis and |
| 16 | 147 | intracellular transport |
| 17 | 148 | Screen for modulators of actin polymerization; diagnostic for |
| | | diseases and conditions associated with actin polymerization (e.g., |
| | | metastasis, immune defects, fertility disorders, aberrant cell division, |
| | | erythrocyte abnormalities) |
| 18 | 149 | In vitro screen for modulators of Ca-ATPase activity; diagnosis of |
| | | disorders of cellular calcium metabolism involving Ca-ATPase |
| | | expression and/or activity |
| 19, 20 | 150, 151 | Diagnostic for disorders involving the mitochondrial ATPase |
| | | complex; in vitro screen for modulators of proton-driven ATP |
| | | synthesis; devices that utilize proton transport coupled to energy |
| | | production |
| 21 | 152 | Diagnostic for psoriasis; reagents for monitoring effectiveness of |
| | | therapy of psoriasis; in vitro screen for therapeutic agents effective |
| | | in psoriasis; in vitro screen for modulators of retinoic acid-sensitive |
| | | transcriptional processes; transgenic animal models for studying |
| | | tissue-specific functions of the protein. |
| 22 | 153 | Detection of bovine leukemia virus; diagnostic for bovine leukemia; |
| | | in vitro screen for agents that interfere with cellular binding and |
| | | uptake of bovine leukemia virus; therapeutic compositions. |
| 23 | 154 | Altering secretion of milk fat droplets by modulating expression of |
| | | the protein in mammary tissue; in vitro screen for modulators of |
| | | secretion; use in fusion proteins to identify animal sources of milk |
| | | production (e.g., genetically modified animals used as bioreactors) |
| 24 | 155 | Assay for collagenolytic activity; in vitro screen for modulators of |
| | | collagenolytic activity; diagnostic for disease processes associated |
| | | with extracellular matrix degradation, (e.g., metastasis, cell migration |
| | | and proliferation disorders) |
| 25 | 156 | Recombinant production of the protein, antibodies and oligo probes |
| | | for detection of expression and development of novel protein |
| | | synthesis inhibitors |
| 26 | 157 | Recombinant production of the protein, antibodies and oligo probes |
| | | for detection of expression and development of novel protein |
| | | synthesis inhibitors |
| 27 | 158 | Diagnostic test for Bardet-Biedl Syndrome (BBS); reagents for |
| | | studying role of MYO 9A in disease pathogenesis; in vitro screen |
| | | for therapeutic agents. |
| 28 | 159 | Diagnostic for mutations affecting transcriptional regulation |
| | | involved in oncogenesis; in vitro screen for antagonists to treat or |
| | | prevent cell proliferation disorders; regulation of CCR4-associated |
| | | factor 1 levels in cells with defects in CCR4-mediated transcription. |
| 29 | 160 | Recombinant production of the protein for use in screen for |
| | | inhibitors of cellular processes involving cathepsin S |
| 30 | 161 | In vitro screen for modulators of caveolae function that may be used |
| | | to prevent and treat diseases caused by intracellular pathogens |
| 31 | 162 | Diagnostic for male fertility disorders; in vitro screen for modulators |
| | | of CRISP-3 expression. |
| 32 | 163 | Tissue regeneration and wound healing compositions; use in in yitro |
| | | tissue engineering to stimulate connective tissue growth; use in |
| | | screening for inhibitors of restenosis; large-scale recombinant |
| | | production of connective tissue growth factor; production of |
| | | antibodies for use in detecting and/or modulating connective tissue |
| | | growth factor-mediated processes. |
| 33 | 164 | In vitro screen for immunosuppressive drugs, inhibitors of HIV-1 |
| | | replication, modulators of c-myb transcription in mammalian cells, |
| | | heat-shock responses |
| 34 | 165 | In vitro screen for agents that inhibit mRNA processing; monitor |
| | | tissue expression of the factor. |
| 35 | 166 | Detection of over-expression of the gene in neuroectodermal tissues; |
| | | in vitro screen for agents that modulate activity and/or expression of |
| | | DEAD-box protein 1 for the treatment of disorders of cell |
| | | proliferation; treatment of tumor progression in neuroblastoma and |
| | | retinoblastoma. |
| 36 | 167 | Recombinant production of quinone reductase 2 for industrial uses |
| | | (detoxification of quinones); in vitro cell-based assay for |
| | | compounds that protect against the toxic and carcinogenic effects of |
| | | quinones via elevating QR2; assay to detect pro-oxidant |
| | | environmental pollutants that alter the expression of QR2; uses of |
| | | QR2 as a putative melatonin receptor, e.g., to modulate circadian |
| | | rhythm and as a screen for modulators of melatonin binding; screen |
| | | for endogenous cellular modulators of transcription of QR2; |
| | | genotyping polymorphic forms of QR2. |
| 37 | 168 | Use of dynein-ATPase screens to detect, isolate and characterize |
| | | modulators of sperm motility and to predict animal fertility; |
| | | diagnosis of secretory disorders associated with vesicle transport. |
| 38 | 169 | Iin vitro screen for selective inhibitors of protein synthesis; use in in |
| 39 | 170 | vitro protein synthesis. |
| 40 | 171 | Use in expression systems for producing correctly folded |
| | | heterologous disulfide bond-containing proteins in bacteria and non- |
| | | human hosts, particularly in milk of transgenic mammals. |
| 41 | 172 | Diagnostic marker to distinguish liposarcoma from benign adipose |
| | | tissue tumors and benign and malignant soft tissue tumors; induction |
| | | of mRNA for adipocyte fatty acid binding protein in human |
| | | monocytes and monocyte cell lines can be used to screen for natural |
| | | and synthetic peroxisome-proliferation activated receptor gamma |
| | | (PPARγ) agonists. |
| 42 | 173 | Recombinant production of MDGI and fragments thereof for |
| | | inhibiting growth and inducing differentiation of mammary cells; |
| | | use of polynucleotide to modulate the amount of MDGI in |
| | | mammary tissue and to increase milk protein synthesis; use of H- |
| | | FABP to modulate beta-adrenergic responses in cardiac muscle, and |
| | | for early detection of acute MI; in vitro screens for modulators, of |
| | | MDGI binding. |
| 43 | 174 | Correction of iron storage defects attributable to ferritin H in |
| | | mammary tissue |
| 44 | 175 | Diagnostic reagents (nucleotide probes or antibodies) and screen for |
| | | mental retardation associated with FXR1 deficiency; expression |
| | | constructs for production of FXR1 (e.g., in milk) of transgenic non- |
| | | human mammals |
| 45 | 176 | In vitro assay for DNA-damaging agents and detection of growth |
| | | arrested cells |
| 46 | 177 | Diagnostic test for diseases involving galactose metabolism |
| 47 | 178 | In vitro screens for modulators of PKC activity and PKC-mediated |
| | | signaling |
| 48 | 179 | Recombinant production of the protein for identifying the Ig class of |
| 49 | 180 | an antibody in a biological specimen and quantitating the amount of |
| | | the antibody in the specimen. |
| 50 | 181 | Bioassay for adipogenic factors based on monitoring enzyme |
| | | induction in cells exposed to the factors; increasing oil yield in |
| | | transgenic crop plants |
| 51 | 182 | Recombinant production of the chemokine; in vitro screen for |
| | | modulators of neutrophil chemotaxis; coadministering with specific |
| | | antigens for immunomodulatory activity. |
| 52 | 183 | Screen for modulators of drug resistance, diagnosis and treatment of |
| | | diseases associated with cell proliferation, cancer, immune response, |
| | | mutagenicity screens. |
| 53 | 184 | Recombinant production of the protein |
| 54 | 185 | Recombinant production of the protein |
| 55 | 186 | In vitro screen for specific inhibitors of initiation of protein |
| | | synthesis |
| 56 | 187 | Recombinant production of the protein for identifying the Ig class of |
| | | an antibody in a biological specimen and quantitating the amount of |
| | | the antibody in the specimen. |
| 57 | 188 | Recombinant production of the protein for identifying the Ig class of |
| | | an antibody in a biological specimen and quantitating the amount of |
| | | the antibody in the specimen. |
| 58 | 189 | Recombinant production of the enzyme and preparation of |
| | | antibodies for detecting the presence and amount of the enzyme in a |
| | | biological sample; assay for viability of in vitro cardiac myocyte |
| | | cultures |
| 59 | 190 | Diagnostic test for type 1 hyperlipoproteinemia |
| 60 | 191 | Replacement therapy in patients lacking the protein; detection and |
| | | diagnosis of mutations that affect androgenic steroid binding |
| 61 | 192 | Recombinant production of the protein for identifying the Ig class of |
| | | an antibody in a biological specimen and quantitating the amount of |
| | | the antibody in the specimen |
| 62 | 193 | Diagnostic for familial hereditary non-polyposis colon cancer |
| 63 | 194 | Iin vitro screen for specific modulators of Golgi nucleoside transport |
| 64 | 195 | Recombinant production of the protein for identifying the Ig class of |
| | | an antibody in a biological specimen and quantitating the amount of |
| | | the antibody in the specimen; marker for B lymphocyte |
| | | differentiation |
| 65 | 196 | Large scale recombinant production of purified enzyme, which can |
| | | be genetically engineered to be soluble and secreted; in vitro screen |
| | | for modulators of enzyme activity; protein glycosylation |
| 66 | 197 | In vitro screen for modulators of protein synthesis; preparation of |
| | | antibodies for use in assessment of malignant potential of lymphoid |
| | | tumors or epithelial tumors based on immunolocalization of staining |
| | | and immunostaining; marker for proliferating lymphocytes and |
| | | growth-factor induced mitogenesis |
| 67 | 198 | Recombinant production of nucleotide integrase for gene therapy |
| | | and genetic marking of cells; detection of LINE-1 elements in |
| | | genome of an organism |
| 68 | 199 | Modulate expression of JAK-STAT signaling in cells involved in |
| | | immediate type 1 hypersensitivity reactions; in vitro screen for |
| | | modulators of JAK-STAT signaling, e.g., in developing lymphoid |
| | | cells and in tumors. |
| 69 | 200 | In vitro assay for selective inhibitors of protein synthesis; cell based |
| | | screen for modulators of expression of EF1 |
| 70 | 201 | In vitro screen for modulators of cationic amino acid transport, e.g., |
| | | for therapeutic regulation of nitric oxide synthesis |
| 71 | 202 | Detection of gene mutations affecting DNA recombination and |
| | | repair, and meiosis |
| 72 | 203 | Iin vitro screen for modulators of EGFR internalization; |
| | | overexpression of SNX1 as a potential antiproliferative therapy to |
| | | downregulate in cancer cells that overexpress EGFR (e.g., breast or |
| | | glial cell tumors) |
| 73 | 204 | Method for detecting sperm production and male fertility using |
| | | antibodies to sperm specific protein |
| 74 | 205 | In vitro assay to quantitate HSP level; in vitro screen for modulators |
| | | of HSP induction; cytoprotection of ex vivo and in vivo cells; in vivo |
| | | modulation of heat shock response in stressed, traumatized and |
| | | aging tissues; preparation of HSP-peptide complexes for use in |
| | | vaccines against specific cancers and infectious diseases. |
| 75 | 206 | Preparation of probes and antibodies for detection of the protein in |
| | | biological samples |
| 76 | 207 | Iin vitro screen for specific protein inhibitors |
| 77 | 208 | Recombinant production of protein for glycosylation of glycolipids |
| | | and glycoproteins |
| 78 | 209 | Construction of in vitro cell models for Alzheimer's disease (see, |
| | | e.g., U.S. Pat. No. 5,994,084); screen for inhibitors of signal |
| | | transduction pathways involved in proliferation, cell cycle control, |
| | | differentiation, and autoimmune diseases such as rheumatoid |
| | | arthritis (see, e.g., U.S. Pat. No. 6,098,631) |
| 79 | 210 | Preparation of probe for detecting expression of certain |
| | | transcriptional coactivators and histone acetyltransferases containing |
| | | the bromodomain motif; immunolocalization reagents |
| 80 | 211 | In vitro modulators of spliceosome function; potential use to target |
| | | appropriately capped nucleic acids to the nucleus of eukaryotic cells |
| | | and parasites. |
| 81 | 212 | protein expression arrays |
| 82 | 213 | Protein expression arrays |
| 83 | 214 | Diagnostic for Alzheimer's beta-amyloid precursor protein |
| 84 | 215 | Marker for hematolymphoid, neuroectodermal and mesenchymal |
| | | tumor cell lines; in vitro screen for inhibitors of lymphoma growth |
| 86 | 217 | In vitro screen for modulators of ornithine decarboxylase (ODC) |
| | | degradation; agonists are candidates for use in anticancer therapy in |
| | | combination with inhibitors of polyamine uptake. |
| 87 | 218 | Iin vitro repair of mitochondrial protein defects; in vitro studies of |
| | | mitochondrial function |
| 88 | 219 | Large scale recombinant production of purified enzyme, which can |
| | | be genetically engineered for solubility and secretion; in vitro screen |
| | | for modulators of enzyme activity; protein glycosylation |
| 89 | 220 | In vitro assay for factors regulating protein import into peroxisomes |
| 90 | 221 | Recombinant production of the purified enzyme for use as catalyst; |
| | | production of lysophosphatidic acid cell signaling molecules. |
| 91 | 222 | Selectable marker for use in HGPRT-negative cells; in vitro screen |
| | | for inhibitors of purine nucleoside phosphorylase. |
| 92 | 223 | Diagnostic for defects in collagen organization in extracellular |
| | | matrix of soft tissues; use in assembly of extracellular matrix for |
| | | replacement tissues |
| 93 | 224 | In vitro screen for modulation of TCR-mediated signaling in |
| | | immune system developmental and pathological processes. |
| 94 | 225 | Recombinant production of the protein for conjugation to drugs that |
| | | act on blood-borne targets; therapy of inflammatory disorders |
| 95 | 226 | Modulation of transcription mediated by the androgen receptor and |
| | | PPAR gamma; in vitro screen for agents that modulate binding of |
| | | the coactivator protein to PPAR gamma, which are candidates for |
| | | regulating the transcription activating effects of PPAR gamma. |
| 96 | 227 | Identification of morphogen analogs using receptor-reporter |
| | | constructs; reconstitution of matrix with cells expressing the BMPR- |
| | | II and osteogenic protein for repair and regeneration of joints, bone |
| | | remodeling, etc. |
| 97 | 228 | Recombinant production of fatty acid synthase; genetic engineering |
| | | of fatty acid metabolic pathways in various organisms that lack |
| | | multifunctional fatty acid synthase; detection of elevated levels of |
| | | fatty acid synthase as a marker for advanced prostate cancer and |
| | | likelihood of recurrence of breast cancer; transgenic animal models |
| | | for hypertriglyceridemia. |
| 98 | 229 | Recombinant production of mammalian factor for use, e.g., in |
| | | detection of binding sites in tissues, and preparation of antibodies |
| | | for detection of the factor; use of the factor for treatment of |
| | | inflammatory bowel disease and peptic ulcers. |
| 99 | 230 | Probes, DNA constructs and antibodies for use in studies of TGF- |
| | | beta signaling pathways |
| 100 | 231 | Probes for detecting overexpression associated with type II diabetes |
| | | and other disease conditions |
| 101 | 232 | In vitro screen for modulators of protein translocation, particularly |
| | | for enhancers of milk protein secretion |
| 102 | 233 | Probes and antibodies to monitor expression of Fto in during normal |
| | | and abnormal tissue development |
| 103 | 234 | Use in preparing MHC class II complexes with autoantigens to treat |
| | | allergic responses, immunological disorders and autoimmune |
| | | diseases in mammals. |
| 104 | 235 | uUe of expression constructs, probes and antibodies to study cell |
| | | cycle regulation and functions of TLK2. |
| 105 | 236 | Diagnostic for diseases of cell proliferation involving activation of |
| | | ETS2 expression; screen for mitogens; screen for modulators of |
| | | ETS2 transcriptional regulation of cellular genes involved in cell |
| | | proliferation (e.g., c-fos, jun-B, c-myc); suppression of |
| | | tumorigenicity in cells not expressing endogenous gene. |
| 106 | 237 | Introduce and express gene in tumor cells for suppression of tumor |
| | | growth; genotyping and detection of mutations |
| 108 | 239 | Diagnostic test for Bardet-Biedl Syndrome (BBS); reagents for |
| | | studying role of MYO 9A in disease pathogenesis; in vitro screen |
| | | for therapeutic agents. |
| 111 | 242 | Monitor expression of the protein for onset of insulin dependent |
| | | diabetes mellitus |
| 112 | 243 | Genetic test for familial British dementia |
| 113 | 244 | Monitor expression associated with juvenile osteosarcoma and |
| | | AIDS; antibodies or sarcolectin fragments for inhibition of |
| | | hyperproliferative disorders; screen for co-activators of sarcolectin- |
| | | stimulated DNA synthesis; modulation of sarcolectin expression in |
| | | cells for enhancement of IFN activity, particularly antiviral effects. |
| 115 | 246 | Expression profiling for transcription factors |
| 116 | 247 | Regulation of secretion of proteins from mammary gland; |
| | | replacement of defective ARF-GEP; diagnostic for GEP defects |
| | | associated with abnormal secretory activity |
| 117 | 248 | Development of cell systems for detection of carcinogens that |
| | | induce DNA strand breaks; screen for anticarcinogens; screen for |
| | | inhibitors of PADPRT for treatment of inflammation, inflammatory |
| | | disorders, arthritis, Gram− and Gram+ endotoxin symptoms of |
| | | systemic infections, cancer and viral infections; use of inhibitors to |
| | | radiosensitize hypotoxic tumor cells and to prevent recovery of |
| | | tumor cells from potentially lethal damage to DNA after radiation |
| | | therapy. |
| 119 | 249 | In vitro screen for selective modulators of protein synthesis |
| 120 | 251 | Probes for detecting overexpression associated with type II diabetes |
| | | and other disease conditions |
| 121 | 252 | Iin vitro screen for modulation of pre-mRNA processing |
| 122 | 253 | Diagnostic for TC21 defects associated with ovarian tumors; in vitro |
| | | screen for modulators of growth inhibition and for modulators of |
| | | GTPase activity. |
| 123 | 254 | Modulation of snRNP and RNA processing |
| 124 | 255 | Detection of selenium deficiency, e.g. in livestock, which produces |
| | | degeneration of skeletal and cardiac muscle |
| 125 | 256 | Preparation of antibodies against recombinant protein for |
| | | immunolocalization studies; monitoring expression in tissues using |
| | | oligo probes |
| 126 | 257 | In vitro screen for defects in ER-calcium interactions; use in in vitro |
| | | reconstituted systems to study regulation of ER biogenesis |
| 127 | 258 | Preparation of antibodies and probes for detecting changes in levels |
| | | of expression in stressed cells |
| 128 | 259 | In vitro screen for modulators of drug metabolism and identification |
| | | of phenol sulfotransferase substrates; screen for enzyme mutations |
| | | affecting drug conjugation activity |
| 129 | 260 | In vitro screen for factors that affect transcriptional regulation by |
| | | TFII-31 |
| 130 | 261 | Recombinant protein expression and preparation of antibodies |
| 131 | 262 | In vitro screen for small molecules that compete for binding to the |
| | | ligand binding domain of the thyroid receptor and act as agonists or |
| | | antagonists of receptor function. |

Isolated polynucleotides of the present invention include the polynucleotides identified herein as SEQ ID NOS: 1-131; polynucleotides comprising a polynucleotide sequence selected from the group consisting of SEQ ID NOS 1-131; polynucleotides comprising at least a specified number of contiguous residues (x-mers) of any of the polynucleotides identified as SEQ ID NOS: 1-131; polynucleotides comprising a polynucleotide sequence that is complementary to any of the above polynucleotides; polynucleotides comprising a polynucleotide sequence that is a reverse sequence or a reverse complement of any of the above polynucleotides; antisense sequences corresponding to any of the above polynucleotides; and other variants of any of the above polynucleotides, such as percentage identity and expectation value variants, as described in this specification.

Variants of polynucleotides and polypeptides of the present invention, such as percentage identity and expectation value variants, have substantially similar functional properties and utilities as those described herein with reference to the specified polynucleotide and/or polypeptide.

The definition of the terms “complement,” “reverse complement,” and “reverse sequence,” as used herein, is best illustrated by the following example. For the sequence 5′ AGGACC 3′, the complement, reverse complement, and reverse sequence are as follows:

| | |
|---|---|
| complement | 3′ TCCTGG 5′ |
| reverse complement | 3′ GGTCCT 5′ |
| reverse sequence | 5′ CCAGGA 3′. |

Preferably, sequences that are complements of a specifically recited polynucleotide sequence are complementary over the entire length of the specific polynucleotide sequence.

As used herein, the term “oligonucleotide” refers to a relatively short segment of a polynucleotide sequence, generally comprising between 6 and 60 nucleotides, and comprehends both probes for use in hybridization assays and primers for use in the amplification of DNA by polymerase chain reaction.

As used herein, the term “polynucleotide” means a single or double-stranded polymer of deoxyribonucleotide or ribonucleotide bases and includes DNA and RNA molecules, both sense and anti-sense strands. The term comprehends cDNA, genomic DNA, recombinant DNA, and wholly or partially synthesized nucleic acid molecules. A polynucleotide may consist of an entire gene, or a portion thereof. A gene is a DNA sequence that codes for a functional protein or RNA molecule. Operable anti-sense polynucleotides may comprise a fragment of the corresponding polynucleotide, and the definition of “polynucleotide” therefore includes all, operable anti-sense fragments. Anti-sense polynucleotides and techniques involving anti-sense polynucleotides are well known in the art and are described, for example, in Robinson-Benion et al., “Anti-sense techniques,” Methods in Enzymol. 254(23):363-375, 1995; and Kawasaki et al., Artific. Organs 20(8):836-848, 1996.

Identification of genomic DNA and heterologous species DNA can be accomplished by standard DNA/DNA hybridization techniques, under appropriately stringent conditions, using all or part of a polynucleotide sequence as a probe to screen an appropriate library. Alternatively, PCR techniques using oligonucleotide primers that are designed based on known genomic DNA, cDNA and protein sequences can be used to amplify and identify genomic and/or cDNA sequences. Synthetic polynucleotides corresponding to the identified sequences, and variants thereof, may be produced by conventional synthesis methods. All the polynucleotides provided by the present invention are isolated and purified, as those terms are commonly used in the art.

The polynucleotide sequences identified as SEQ ID NOS: 1-131 were derived from bovine mammary gland cells. Certain of the polynucleotides of the present invention may be “partial” sequences, in that they do not represent a full-length gene encoding a full-length polypeptide. Such partial sequences may contain ORFs or partial ORFs, and may be extended by analyzing and sequencing various DNA libraries using primers and/or probes and well known hybridization and/or PCR techniques. The sequences identified as SEQ ID NOS: 1-131 may thus be extended until a full open reading frame encoding a polypeptide, a full-length polynucleotide and/or gene capable of expressing a polypeptide, or another useful portion of the genome is identified. Such extended sequences, including full-length polynucleotides and genes, are described as “corresponding to” a sequence identified as one of the sequences of SEQ ID NOS: 1-131, or a variant thereof, or a portion of one of the sequences of SEQ ID NOS: 1-131, or a variant thereof, when the extended polynucleotide comprises an identified sequence or its variant, or an identified contiguous portion (x-mer) of one of the sequences of SEQ ID NOS: 1-131 or a variant thereof.

The polynucleotides identified as SEQ ID NOS: 1-131 were isolated from bovine mammary gland EDNA libraries and represent sequences that are expressed in the tissue from which the cDNA was prepared. The sequence information may be used to isolate or synthesize expressible DNA molecules, such as open reading frames or full-length genes, that then can be used as expressible or otherwise functional DNA in cows and other organisms. Similarly, RNA sequences, reverse sequences, complementary sequences, antisense sequences, and the like, corresponding to the polynucleotides of the present invention, may be routinely ascertained and obtained using the cDNA sequences identified as SEQ ID NOS: 1-131.

The polynucleotides identified as SEQ ID NOS: 1-131 contain open reading frames (“ORFs”) or partial open reading frames encoding polypeptides. Additionally, open reading frames encoding polypeptides may be identified in extended or full-length sequences corresponding to the sequences set out as SEQ ID NOS: 1-131. Open reading frames may be identified using techniques that are well known in the art. These techniques include, for example, analysis for the location of known start and stop codons, most likely reading frame identification based on codon frequencies, etc. Suitable tools and software for ORF analysis are available, for example, on the Internet. Additional tools and software for ORF analysis include GeneWise, available from The Sanger Center, Wellcome Trust Genome Campus, Hinxton, Cambridge, CB10 1SA, United Kingdom; Diogenes, available from Computational Biology Centers, University of Minnesota, Academic Health Center, UMHG Box 43 Minneapolis Minn. 55455; and GRAIL, available from the Informatics Group, Oak Ridge National Laboratories, Oak Ridge, Tennessee Tenn. Open reading frame's and portions of open reading frames may be identified in the polynucleotides of the present invention. Once a partial open reading frame is identified, the polynucleotide may be extended in the area of the partial open reading frame using techniques that are well known in the art until the polynucleotide for the full open reading frame is identified. Thus, polynucleotides and open reading frames encoding polypeptides may be identified using the polynucleotides of the present invention.

Once open reading frames are identified in the polynucleotides of the present invention, the open reading frames may be isolated and/or synthesized. Expressible genetic constructs comprising the open reading frames and suitable promoters, initiators, terminators, etc., which are well known in the art, may then be constructed. Such genetic constructs may be introduced into a host cell to express the polypeptide encoded by the open reading frame. Suitable host cells may include various prokaryotic and eukaryotic cells, including mammalian cells. In vitro expression of polypeptides is also possible, as well known in the art.

Polypeptides encoded by the polynucleotides of the present invention may be expressed and used in various assays to determine their biological activity. Such polypeptides may be used to raise antibodies, to isolate corresponding interacting proteins or other compounds, and to quantitatively determine levels of interacting proteins or other compounds.

In another aspect, the present invention provides isolated polypeptides encoded, or partially encoded, by the above polynucleotides. As used herein, the term “polypeptide” encompasses amino acid chains of any length, including full-length proteins, wherein the amino acid, residues are linked by covalent peptide bonds. The term “polypeptide encoded by a polynucleotide” as used herein, includes polypeptides encoded by a polynucleotide that comprises an isolated polynucleotide sequence or variant provided herein. Polypeptides of the present invention may be naturally purified products, or may be produced partially or wholly using recombinant techniques. Such polypeptides may be glycosylated with bacterial, fungal, mammalian or other eukaryotic carbohydrates or may be non-glycosylated. In specific embodiments, the inventive polypeptides comprise an amino acid sequence selected from the group consisting of SEQ ID NO: 132-262.

Polypeptides of the present invention may be produced recombinantly by inserting a polynucleotide sequence that encodes the polypeptide into a genetic construct and expressing the polypeptide in an appropriate host. Any of a variety of genetic constructs known to those of ordinary skill in the art may be employed. Expression may be achieved in any appropriate host cell that has been transformed or transfected with a genetic construct containing a polynucleotide that encodes a recombinant polypeptide. Suitable host cells include prokaryotes, yeast, and higher eukaryotic cells. Preferably, the host cells employed are Escherichia coli, insect, yeast, or a mammalian cell line such as COS or CHO. The polynucleotide sequences expressed in this manner may encode naturally occurring polypeptides, portions of naturally occurring polypeptides, or other variants thereof.

In a related aspect, polypeptides are provided that comprise at least a functional portion of a polypeptide having an amino acid sequence encoded by a polynucleotide of the present invention. As used herein, the “functional portion” of a polypeptide is that portion which contains the active site essential for affecting the function of the polypeptide, for example, the portion of the molecule that is capable of binding one or more reactants. The active site may be made up of separate portions present on one or more polypeptide chains and will generally exhibit high binding affinity.

Functional portions of a polypeptide may be identified by first preparing fragments of the polypeptide by either chemical or enzymatic digestion of the polypeptide, or by mutation analysis of the polynucleotide that encodes the polypeptide and subsequent expression of the resulting mutant polypeptides. The polypeptide fragments or mutant polypeptides are then tested to determine which portions retain biological activity, using, for example, the representative assays provided below.

Portions and other variants of the inventive polypeptides may also be generated by synthetic or recombinant means. Synthetic polypeptides having fewer than about 100 amino acids, and generally fewer than about 50 amino acids, may be generated using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2154, 1963. Equipment for automated synthesis of polypeptides is commercially available from suppliers such, as Perkin Elmer/Applied BioSystems, Inc. (Foster City, Calif.), and may be operated according to the manufacturer's instructions. Variants of a native polypeptide may be prepared using standard mutagenesis techniques, such as oligonucleotide-directed, site-specific mutagenesis (Kunkel, Proc. Natl. Acad. Sci. USA 82:488-492, 1985). Sections of polynucleotide sequence may also be removed using standard techniques to permit preparation of truncated polypeptides.

In general, the polypeptides disclosed herein are prepared in an isolated, substantially pure, form. Preferably, the polypeptides are at least about 80% pure, more preferably at least about 90% pure, and most preferably at least about 99% pure. In certain embodiments, described in detail below, the isolated polypeptides are incorporated into pharmaceutical compositions or vaccines.

As used herein, the term “variant” comprehends nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 50%, more preferably at least 75%, more preferably yet at least 90% or 95%, and most preferably, at least 98% identity to a sequence of the present invention. The percentage identity is determined by aligning the two sequences to be compared as described below, determining the number of identical residues in the aligned portion, dividing that number by the total number of residues in the inventive (queried) sequence, and multiplying the result by 100. By way of example only, assume a queried polynucleotide having 220 nucleic acids has a hit to a polynucleotide sequence in the EMBL database having 520 nucleic acids over a stretch of 23 nucleotides in the alignment produced by the BLASTN algorithm using the default parameters as described below. The 23 nucleotide hit includes 21 identical nucleotides, one gap and one different nucleotide. The percentage identity of the queried polynucleotide to the hit in the EMBL database is thus 21/220 times 100, or 9.5%. The percentage identity of polypeptide sequences may be determined in a similar fashion.

Polynucleotide and polypeptide sequences may be aligned, and percentages of identical residues in a specified region may be determined against another polynucleotide or polypeptide, using computer algorithms that are publicly available. Two exemplary algorithms for aligning and identifying the similarity of polynucleotide sequences are the BLASTN and FASTA algorithms. Polynucleotides may also be analyzed using the BLASTX algorithm, which compares the six-frame conceptual translation products of a nucleotide query sequence (both strands) against a protein sequence database. The percentage identity of polypeptide sequences may be examined using the BLASTP algorithm. The BLASTN, BLASTP and BLASTX algorithms are available on the NCBI anonymous FTP server and from the National Center for Biotechnology Information (NCBI), National Library of Medicine, Building 38A, Room 8N805, Bethesda, Md. 20894, USA. The BLASTN algorithm Version 2.0.4 [Feb. 24, 1998], Version 2.0.6 [Ser. 16, 1998] and Version 2.0.11 [Jan. 20, 2000], set to the parameters described below, is preferred for use in the determination of polynucleotide variants according to the present invention. The BLASTP algorithm, set to the parameters described below, is preferred for use in the determination of polypeptide variants according to the present invention. The use of the BLAST family of algorithms, including BLASTN, BLASTP and BLASTX, is described at NCBI's website and in the publication of Altschul, et al., Nucleic Acids Res. 25:3389-3402, 1997.

The FASTA and FASTX algorithms are available on the Internet, and from the University of Virginia by contacting David Hudson, Vice Provost for Research, University of Virginia, P.O. Box 9025, Charlottesville, Va. 22906-9025, USA. The FASTA algorithm, set to the default parameters described in the documentation and distributed with the algorithm, may be used in the determination of polynucleotide variants. The readme files for FASTA and FASTX Version 1.0× that are distributed with the algorithms describe the use of the algorithms and describe the default parameters. The use of the FASTA and FASTX algorithms is described in Pearson and Lipman, Proc. Natl. Acad. Sci. USA 85:2444-2448, 1988; and Pearson, Methods in Enzymol. 183:63-98, 1990.

The following running parameters are preferred for determination of alignments and similarities using BLASTN that contribute to the E values and percentage identity for polynucleotides. Unix running command with the following default parameters blastall -p blastn -d embldb -e 10 -G 0 -E 0 -r 1 -v 30 -b 30 -i queryseq -o results; and parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -r Reward for a nucleotide match (blastn only) [Integer]; -v Number of one-line descriptions (V) [Integer]; -b Number of alignments to show (B) [Integer]; -i Query File [File In]; -o BLAST report Output File [File Out] Optional.

The following running parameters are preferred for determination of alignments and similarities using BLASTP that contribute to the E values and percentage identity of polypeptide sequences: blastall -p blastp -d swissprotdb -e 10 -G 0 -E 0 -v 30 -b 30 -i queryseq -o results; the parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -v Number of one-line descriptions (v) [Integer]; -b Number of alignments to show (b) [Integer]; -I Query File [File In]; -o BLAST report Output File [File Out] Optional.

The “hits” to one or more database sequences by a queried sequence produced by BLASTN, BLASTP, FASTA, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, FASTA and BLASTP algorithms also produce “Expect” values for polynucleotide and polypeptide alignments. The Expect value (E) indicates the number of hits one can “expect” to see over a certain number of contiguous sequences by chance when searching a database of a certain size. The Expect value is used as a significance threshold for determining whether the hit to a database indicates true similarity. For example, an E value of 0.1 assigned to a polynucleotide hit is interpreted as meaning that in a database of the size of the EMBL database, one might expect to see 0.1 matches over the aligned portion of the sequence with a similar score simply by chance. By this criterion, the aligned and matched portions of the sequences then have a probability of 90% of being related. For sequences having an E value of 0.01 or less over aligned and matched portions, the probability of finding a match by chance in the EMBL database is 1% or less using the BLASTN algorithm. E values for polypeptide sequences may be determined in a similar fashion using various polypeptide databases, such as the SwissProt database.

According to one embodiment, “variant” polynucleotides and polypeptides, with reference to each of the polynucleotides and polypeptides of the present invention, preferably comprise sequences having the same number or fewer nucleic or amino acids than each of the polynucleotides or polypeptides of the present invention and producing an E value of 0.01 or less when compared to the polynucleotide or polypeptide of the present invention. That is, a variant polynucleotide or polypeptide is any sequence that has at least a 99% probability of being the same as the polynucleotide or polypeptide of the present invention, measured as having an E value of 0.01 or less using the BLASTN, FASTA or BLASTP algorithms set at the default parameters. According to a preferred embodiment, a variant polynucleotide is a sequence having the same number or fewer nucleic acids than a polynucleotide of the present invention that has at least a 99% probability of being the same as the polynucleotide of the present invention, measured as having an E value of 0.01 or less using the BLASTN algorithm set at the default parameters. Similarly, according to a preferred embodiment, a variant polypeptide is a sequence having the same number or fewer amino acids than a polypeptide of the present invention that has at least a 99% probability of being the same as the polypeptide of the present invention, measured as having an E value of 0.01 or less using the BLASTP algorithm set at the default parameters.

In addition to having a specified percentage identity to an inventive polynucleotide or polypeptide sequence, variant polynucleotides and polypeptides preferably have additional structure and/or functional features in common with the inventive polynucleotide or polypeptide. Polypeptides having a specified degree of identity to a polypeptide of the present invention share a high degree of similarity in their primary structure and have substantially similar functional properties. In addition to sharing a high degree of similarity in their primary structure to polynucleotides of the present invention, polynucleotides having a specified degree of identity to, or capable of hybridizing to an inventive polynucleotide preferably have at least one of the following features: (i) they contain an open reading frame or partial open reading frame encoding a polypeptide having substantially the same functional properties as the polypeptide encoded by the inventive polynucleotide; or (ii) they contain identifiable domains in common.

Alternatively, variant polynucleotides hybridize to a polynucleotide of the present invention, or a complement thereof, under stringent conditions. As used herein, “stringent conditions” refers to prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C., and two washes of 30 minutes each in 0.2×SSC, 0.1% SDS at 65° C.

The present invention also encompasses polynucleotides that differ from the disclosed sequences but that, as a consequence of the discrepancy of the genetic code, encode a polypeptide having similar enzymatic activity as a polypeptide encoded by a polynucleotide of the present invention. Thus, polynucleotides comprising sequences that differ from the polynucleotide sequences recited in SEQ ID NOS: 1-131 (or complements, reverse sequences, or reverse complements of those sequences) as a result of conservative substitutions are encompassed within the present invention. Additionally, polynucleotides comprising sequences that differ from the inventive polynucleotide sequences or complements, reverse complements, or reverse sequences as a result of deletions and/or insertions totaling less than 10% of the total sequence length are also contemplated by and encompassed within the present invention. Similarly, polypeptides comprising sequences that differ from the inventive polypeptide sequences as a result of amino acid substitutions, insertions, and/or deletions totaling less than 10% of the total sequence length are contemplated by and encompassed within the present invention, provided the variant polypeptide has similar activity to the inventive polypeptide.

The polynucleotides of the present invention may be isolated from various libraries, or may be synthesized using techniques that are well known in the art. The polynucleotides may be synthesized, for example, using automated oligonucleotide synthesizers (e.g., Beckman Oligo 1000M DNA Synthesizer) to obtain polynucleotide segments of up to 50 or more nucleic acids. A plurality of such polynucleotide segments may then be ligated using standard DNA manipulation techniques that are well known in the art of molecular biology. One conventional and exemplary polynucleotide synthesis technique involves synthesis of a single stranded polynucleotide segment having, for example, 80 nucleic acids, and hybridizing that segment to a synthesized complementary 85 nucleic acid segment to produce a 5 nucleotide overhang. The next segment may then be synthesized in a similar fashion, with a 5 nucleotide overhang on the opposite strand. The “sticky” ends ensure proper ligation when the two portions are hybridized. In this way, a complete polynucleotide of the present invention may be synthesized entirely in vitro.

As noted above, certain of the polynucleotides identified as SEQ ID NOS: 1-131 may be referred to as “partial” sequences, in that they may not represent the full coding portion of a gene encoding a naturally occurring polypeptide. Partial polynucleotide sequences disclosed herein may be employed to obtain the corresponding full-length genes for various species and organisms by, for example, screening DNA expression libraries using hybridization probes based on the polynucleotides of the present invention, or using PCR amplification with primers based upon the polynucleotides of the present invention. In this way one can, using methods well known in the art, extend a polynucleotide of the present invention upstream and downstream of the corresponding mRNA, as well as identify the corresponding genomic DNA, including the promoter and enhancer regions, of the complete gene. The present invention thus comprehends isolated polynucleotides comprising a sequence identified in SEQ ID NOS: 1-131, or a variant of one of the specified sequences, that encode a functional polypeptide, including full-length genes. Such extended polynucleotides may have a length of from about 50 to about 4,000 nucleic acids or base pairs, and preferably have a length of less than about 4,000 nucleic acids or base pairs, more preferably yet a length of less than about 3,000 nucleic acids or base pairs, more preferably yet a length of less than about 2,000 nucleic acids or base pairs. Under some circumstances, extended polynucleotides of the present invention may have a length of less than about 1,800 nucleic acids or base pairs, preferably less than about 1,600 nucleic acids or base pairs, more preferably less than about 1,400 nucleic acids or base pairs, more preferably yet less than about 1,200 nucleic acids or base pairs, and most preferably less than about 1,000 nucleic acids or base pairs.

As used herein, the term “x-mer,” with reference to a specific value of “x,” refers to a polynucleotide or polypeptide, respectively, comprising at least a specified number (“x”) of contiguous residues of any of the polynucleotides provided in SEQ ID NOS: 1-131. The value of x may be from about 20 to about 600, depending upon the specific sequence.

Polynucleotides of the present invention comprehend polynucleotides comprising at least a specified number of contiguous residues (x-mers) of any of the polynucleotides identified as SEQ ID NOS: 1-131, or their variants. Polypeptides of the present invention comprehend polypeptides comprising at least a specified number of contiguous residues (x-mers) of any of the polypeptides corresponding to the polynucleotides of SEQ ID NOS: 1-131. According to preferred embodiments, the value of x is at least 20, more preferably at least 40, more preferably yet at least 60, and most preferably at least 80. Thus, polynucleotides of the present invention include polynucleotides comprising a 20-mer; a 40-mer a 60-mer, an 80-mer, a 100-mer, a 120-mer, a 150-mer, a 180-mer, a 220-mer, a 250-mer, a 300-mer, 400-mer, 500-mer or 600-mer of a polynucleotide provided in SEQ ID NOS: 1-131, or a variant of one of the polynucleotides provided in SEQ ID NOS: 1-131. Similarly, polypeptides of the present invention include polypeptides comprising a 20-mer, a 40-mer, a 60-mer, an 80-mer, a 100-mer, a 120-mer, a 150-mer, a 180-mer, a 220-mer, a 250-mer, a 300-mer, 400-mer, 500-mer or 600-mer of an amino acid sequence provided in SEQ ID NO: 132-262 or a variant thereof.

The inventive polynucleotides may be isolated by high throughput sequencing of cDNA libraries prepared from bovine mammary gland tissue as described below in Example 1. Alternatively, oligonucleotide probes and/or primers based on the sequences provided in SEQ ID NOS: 1-131, can be synthesized and used to identify positive clones in either cDNA or genomic DNA libraries from bovine mammary gland cells by means of hybridization or polymerase chain reaction (PCR) techniques. Probes can be shorter than thee sequences provided herein but should be at least about 10, preferably at least about 15 and most preferably at least about 20 nucleotides in length. Hybridization and PCR techniques suitable for use with such oligonucleotide probes are well known in the art (see, for example, Mullis et al., Cold Spring Harbor Symp. Quant. Biol., 51:263, 1987; Erlich, ed., PCR technology, Stockton Press: NY, 1989; and Sambrook et al., in Molecular cloning: a laboratory manual, 2nd ed., CSHL Press: Cold Spring Harbor, N.Y., 1989). Positive clones may be analyzed by restriction enzyme digestion, DNA sequencing or the like.

In addition, polynucleotide sequences of the present invention may be generated by synthetic means using techniques well known in the art. Equipment for automated synthesis of oligonucleotides is commercially available from suppliers such as Perkin Elmer/Applied Biosystems Division (Foster City, Calif.) and may be operated according to the manufacturer's instructions.

Oligonucleotide probes and primers complementary to and/or corresponding to SEQ ID NOS: 1-131, and variants of those sequences, are also comprehended by the present invention. Such oligonucleotide probes and primers are substantially complementary to the polynucleotide of interest. An oligonucleotide probe or primer is described as “corresponding to” a polynucleotide of the present invention, including one of the sequences set out as SEQ ID NOS: 1-131 or a variant thereof, if the oligonucleotide probe or primer, or its complement, is contained within one of the sequences set out as SEQ ID NOS: 1-131 or a variant of one of the specified sequences.

Two single stranded sequences are said to be substantially complementary when the nucleotides of one strand, optimally aligned and compared, with the appropriate nucleotide insertions and/or deletions, pair with at least 80%, preferably at least 90% to95%, and more preferably at least 98% to 100%, of the nucleotides of the other strand. Alternatively, substantial complementarity exists when a first DNA strand will selectively hybridize to a second DNA strand under stringent hybridization conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C. more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents, and extent of base mismatching, the combination of parameters is more important than the absolute measure of any one alone. DNA-DNA hybridization studies may be performed using either genomic DNA or DNA derived by preparing cDNA from the RNA present in the sample.

In addition to DNA-DNA hybridization, DNA-RNA or RNA-RNA hybridization assays are also possible. In the first case, the mRNA from expressed genes would then be detected instead of genomic DNA or cDNA derived from mRNA of the sample. In the second case, RNA probes could be used. In additional artificial analogs of DNA hybridizing specifically to target sequences could also be used.

In specific embodiments, the inventive oligonucleotide probes and/or primers comprise at least about 6 contiguous residues, more preferably at least about 10 contiguous residues, and most preferably, at least about 20 contiguous residues complementary to a polynucleotide sequence of the present invention. Probes and primers of the present invention may be from about 8 to 100 base pairs in length, or preferably from about 10 to 50 base pairs in length, or more preferably from about 15 to 40 abase pairs in length. The probes can be easily selected using procedures well known in the art, taking into account DNA-DNA hybridization stringencies, annealing and melting temperatures, potential for formation of loops, and other factors which are well known in the art. Tools and software suitable for designing probes, and especially suitable for designing PCR primers, are available on the Internet, for example. In addition, a software program suitable for designing probes, and especially for designing PCR primers, is available from Premier Biosoft International, 3786 Corina Way, Palo Alto, Calif. 94303-4504. Preferred techniques for designing PCR primers are also disclosed in Dieffenbach and Dyksler, PCR Primer: a laboratory manual, CSHL Press: Cold Spring Harbor, N.Y., 1995.

A plurality of oligonucleotide probes or primers corresponding to a polynucleotide of the present invention may be provided in a kit form. Such kits generally comprise multiple DNA or oligonucleotide probes, each probe being specific for a polynucleotide sequence. Kits of the present invention may comprise one or more probes or primers corresponding to a polynucleotide of the present invention, including a polynucleotide sequence identified in SEQ ID NOS: 1-131.

In one embodiment useful for high-throughput assays, the oligonucleotide probe kits of the present invention comprise multiple probes in an array format, wherein each probe is immobilized in a predefined, spatially addressable location on the surface of a solid substrate. Array formats which may be usefully employed in the present invention are disclosed, for example, in U.S. Pat. Nos. 5,412,087; 5,545,531, and PCT Publication No. WO 95/00530, the disclosures of which are hereby incorporated by reference.

Oligonucleotide probes for use in the present invention may be constructed synthetically prior to immobilization on an array, using techniques well known in the art (See, for example, Gait, ed., Oligonucleotide synthesis a practical approach, IRL Press: Oxford, England, 1984). Automated equipment for the synthesis of oligonucleotides is available commercially from such companies as Perkin Elmer/Applied Biosystems Division (Foster City, Calif.) and may be operated according to the manufacturer's instructions. Alternatively, the probes may be constructed directly on the surface of the array using techniques taught, for example, in PCT Publication No. WO 95/00530.

The solid substrate and the surface thereof preferably form a rigid support and are generally formed from the same material. Examples of materials from which the solid substrate may be constructed include polymers, plastics, resins, membranes, polysaccharides, silica or silica-based materials carbon, metals and inorganic glasses. Synthetically prepared probes may be immobilized on the surface of the solid substrate using techniques well known in the art, such as those disclosed in U.S. Pat. No. 5,412,087.

In one such technique, compounds having protected functional groups, such as thiols protected with photochemically removable protecting groups, are attached to the surface of the substrate. Selected regions of the surface are then irradiated with a light source, preferably a laser, to provide reactive thiol groups. This irradiation step is generally performed using a mask having apertures at predefined locations using photolithographic techniques well known in the art of semiconductors. The reactive thiol groups are then incubated with the oligonucleotide probe to be immobilized. The precise conditions for incubation, such as temperature, time and pH, depend on the specific probe and can be easily determined by one of skill in the art. The surface of the substrate is washed free of unbound probe and the irradiation step is repeated using a second mask having a different pattern of apertures. The surface is subsequently incubated with a second, different, probe. Each oligonucleotide probe is typically immobilized in a discrete area of less than about 1 mm². Preferably each discrete area is less than about 10,000 mm², more preferably less than about 100 mm². In this manner, a multitude of oligonucleotide probes may be immobilized at predefined locations on the array.

The resulting array may be employed to screen for differences in organisms or samples or products containing genetic maternal as follows. Genomic or cDNA libraries are prepared using techniques well known in the art. The resulting target DNA is then labeled with a suitable marker, such as a radiolabel, chromophore, fluorophore or chemiluminescent agent, using protocols well known for those skilled in the art. A solution of the labeled target DNA is contacted with the surface of the array and incubated for a suitable period of time.

The surface of the array is then washed free of unbound target DNA and the probes to which the target DNA hybridized are determined by identifying those regions of the array to which the markers are attached. When the marker is a radiolabel, such as ³²P, autoradiography is employed as the detection method. In one embodiment, the marker is a fluorophore, such as fluorescein, and the location of bound target DNA is determined by means of fluorescence spectroscopy. Automated equipment for use in fluorescence scanning of oligonucleotide probe arrays is available from Affymetrix, Inc. (Santa Clara, Calif.) and may be operated according to the manufacturer's instructions. Such equipment may be employed to determine the intensity of fluorescence at each predefined location on the array, thereby providing a measure of the amount of target DNA bound at each location. Such an assay would be able to indicate not only the absence and presence of the marker probe in the target, but also the quantitative amount as well.

In this manner, oligonucleotide probe kits of the present invention may be employed to examine the presence/absence (or relative amounts in case of mixtures) of polynucleotides in different samples or products containing different materials rapidly and in a cost-effective manner.

Another aspect of the present invention involves collections of a plurality of polynucleotide sequences of the present invention. A collection of a plurality of the polynucleotides of the present invention, particularly the polynucleotides identified as SEQ ID NOS: 1-131, may be recorded and/or stored on a storage medium and subsequently accessed for purposes of analysis, comparison, etc. Suitable storage media include magnetic media such as magnetic diskettes, magnetic tapes, CD-ROM storage media, optical storage media, and the like. Suitable storage media and methods for recording and storing information, as well as accessing information such as polynucleotide sequences recorded on such media, are well known in the art. The polynucleotide information stored on the storage medium is preferably computer-readable and may be used for analysis and comparison of the polynucleotide information.

Another aspect of the present invention thus involves storage medium on which are recorded a collection of the polynucleotides of the present invention, particularly a collection of the polynucleotides identified as SEQ ID NOS: 1-131. According to one embodiment, the storage medium includes a collection of at least 20, preferably at least 50, more preferably at least 100, and most preferably at least 200 of the polynucleotides of the present invention, preferably the polynucleotides identified as SEQ ID NOS: 1-131, including variants of those polynucleotides.

In another aspect, the present invention provides genetic constructs comprising, in the 5′-3′ direction, a gene promoter sequence; and an open reading frame coding for at least a functional portion of a polypeptide encoded by a polynucleotide of the present invention. In certain embodiments, the genetic constructs of the present invention also comprise a gene, or transcription, termination sequence. The open reading frame may be oriented in either a sense or antisense direction. Genetic constructs comprising a non-coding region of a gene coding for a polypeptide encoded by the above polynucleotides or a nucleotide sequence complementary to a non-coding region, together with a gene promoter sequence, are also provided. A terminator sequence may form part of this construct. Preferably, the gene promoter and termination sequences are functional in a host organism. More preferably, the gene promoter and termination sequences are common to those of the polynucleotide being introduced. The genetic construct may further include a marker for the identification of transformed cells.

Techniques for operatively linking the components of the genetic constructs are well known in the art and include the use of synthetic linkers containing one or more restriction endonuclease sites as described, for example, by Sambrook et al., in Molecular cloning: a laboratory manual, Cold Spring Harbor Laboratories Press: Cold Spring Harbor, N.Y., 1989. The genetic constructs of the present invention may be linked to a vector having at least one replication system, for example, E. coli, whereby after each manipulation, the resulting construct can be cloned and sequenced and the correctness of the manipulation determined.

Transgenic cells comprising the genetic constructs of the present invention are also provided by the present invention, together with organisms comprising such transgenic cells, products and progeny of such organisms. Techniques for stably incorporating genetic constructs into the genome of target organisms are well known in the art.

In one aspect, the present invention provides methods for using one or more of the inventive polypeptides or polynucleotides to treat disorders in a mammal, including a human. In this aspect, the polypeptide or polynucleotide is generally present within a composition, such as an immunogenic composition. Such compositions may comprise one or more polypeptides, each of which may contain one or more of the above sequences (or variants thereof), and a physiologically acceptable carrier. Immunogenic compositions may comprise one or more of the above polypeptides and an immunostimulant, such as an adjuvant, into which the polypeptide is incorporated.

Alternatively, a composition of the present invention may contain a polynucleotide encoding one or more polypeptides as described above, such that the polypeptide is generated in situ. In such compositions, the polynucleotide may be present within any of a variety of delivery systems known to those of ordinary skill in the art, including nucleic acid expression systems, and bacterial and viral expression systems. Appropriate nucleic acid expression systems contain the necessary polynucleotide sequences for expression in a mammal (such as a suitable promoter and terminator signal). Bacterial delivery systems involve the administration of a bacterium (such as Bacillus Calmette-Guerin) that expresses an immunogenic portion of the polypeptide on its cell surface. In a preferred embodiment, the DNA may be introduced using a viral expression system (e.g., vaccinia or other poxvirus, retrovirus, or adenovirus), which may involve the use of a non-pathogenic, or defective, replication competent virus. Techniques for incorporating polynucleotides into such expression systems are well known in the art. The DNA may also be “naked,” as described, for example, in Ulmer et al., Science 259:1745-1749, 1993; and reviewed by Cohen, Science 259:1691-1692, 1993. The uptake of naked DNA may be increasec by coating the DNA onto biodegradable beads, which are efficiently transported into the cells.

Routes and frequency of administration, as well as dosage, will vary from individual to individual. In general, the pharmaceutical compositions and vaccines may be administered by injection (e.g., intradermal, intramuscular, intravenous, or subcutaneous); intranasally (e.g., by aspiration); or orally. In general, the amount of polypeptide present in a dose (or produced in situ by the DNA in a dose) ranges from about 1 pg to about 100 mg per kg of host, typically from about 10 pg to about 1 mg per kg of host, and preferably from about 100 pg to about 1 μg per kg of host. Suitable dose sizes will vary with the size of the mammal, but will typically range from about 0.1 ml to about 5 ml.

While any suitable carrier known to those of “ordinary” skill in the art may be employed in the pharmaceutical compositions of this invention, the type of carrier will vary depending on the mode of administration. For parenteral administration, such as subcutaneous injection, the carrier preferably comprises water, saline, alcohol, a lipid, a wax, or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carrier's for the pharmaceutical compositions of this invention. Suitable biodegradable microspheres are disclosed, for example, in U.S. Pat. Nos. 4,897,268 and 5,075,109.

Any of a variety of immunostimulants may be employed in the immunogenic compositions of this invention to non-specifically enhance the immune response. Most adjuvants contain a substance designed to protect the antigen from rapid catabolism, such as aluminum hydroxide or mineral oil, and a non-specific stimulator of immune responses, such as lipid A, Bordetella pertussis, or Mycobacterium tuberculosis. Suitable adjuvants are commercially available as, for example, Freund's Incomplete Adjuvant and Freund's Complete Adjuvant (Difco Laboratories, Detroit, Mich.), and Merck Adjuvant 65 (Merck and Company, Inc., Rahway, N.J.). Other suitable adjuvants include alum, biodegradable microspheres, monophosphoryl lipid A, and Quil A.

The polypeptides of the present invention may additionally be used in assays to determine biological activity, to raise antibodies, to isolate corresponding ligands or receptors, in assays to quantitatively determine levels of protein or cognate corresponding ligand or receptor, as anti-inflammatory agents, and in compositions for mammary glands, connective tissue and/or nerve tissue growth or regeneration.

The polynucleotides of the present invention may be used for expression in a transgenic animal, as disclosed in U.S. Pat. No. 5,714,345, which teaches the use of transgenic animals capable of expressing a desired protein prepared by introducing into an egg or embryo cell of an animal, an expression construct containing the sequence corresponding at least in part to a specific polynucleotide, which encodes the desired protein. In the same manner, the desired protein corresponding to a selected polynucleotide sequence of the present invention, could be employed in transgenic animals for the production of milk containing the desired protein, as disclosed in U.S. Pat. No. 5,849,992.

In addition, the regulatory sequences contained in the present cDNA sequences, or regulatory sequences isolated by using the present sequences for genome screening and sequencing, as well known in the art, could be used in transgenic animals to direct the expression of a desired gene product according to the nature of the regulatory polynucleotide sequence, in a way similar to that taught in U.S. Pat. No. 5,850,000.

### EXAMPLE 1

### Isolation of cDNA Sequences from Bovine Mammary Gland cDNA Libraries

Bovine mammary gland cDNA expression libraries were constructed and screened as follows. mRNA was extracted from lactating bovine mammary tissue (Jersey breed, late lactating, non-pregnant cow, 2 hours post-milking) using standard protocols. mRNA was precipitated with ethanol and the total RNA preparate was purified using a Poly(A) Quik mRNA Isolation Kit (Stratagene, La Jolla, Calif.). A cDNA expression library was constructed from the purified mRNA by reverse transcriptase synthesis followed by insertion of the resulting cDNA clones in Lambda ZAP using a ZAP Express cDNA Synthesis Kit (Stratagene), according to the manufacturer's protocol. The resulting cDNAs were packaged using a Gigapack II Packaging Extract (Stratagene) employing 1 μl of sample DNA from the 5 μl ligation mix. Mass excision of the library was done using XL1-Blue MRF’ cells and XLOLR cells (Stratagene) with ExAssist helper phage (Stratagene). The excised phagemids were diluted with NZY broth (Gibco BRL, Gaithersburg, Md.) and plated out onto LB-kanamycin agar plates containing 5-bromo-4-chloro-3-indolyl-beta-D-galactoside (X-gal) and isopropylthio-beta-galactoside (IPTG).

Of the colonies plated and picked for DNA preparations, the large majority contained an insert suitable for sequencing. Positive colonies were cultured in NZY broth with kanamycin and cDNA was purified by means of REAL DNA minipreps (Qiagen, Venlo, The Netherlands). Agarose gel at 1% was used to screen sequencing templates for chromosomal contamination. Dye terminator sequences were prepared using a Biomek 2000 robot (Beckman Coulter Inc., Fullerton, Calif.) for liquid handling and DNA amplification using a 9700 PCR machine (Perkin Elmer/Applied Biosystems, Foster City, Calif.) according to the manufacturer's protocol.

The DNA sequences for positive clones were obtained using a Perkin Elmer/Applied Biosystems Division Prism 377 sequencer. cDNA clones were sequenced from the 5′ end. The sequences of the isolated polynucleotides are identified as SEQ ID NOS: 1-131, with the corresponding amino acid sequences being provided in SEQ ID NO: 132-262. The polynucleotides of SEQ ID NO 3, 8, 14, 20, 25, 31, 33, 41-43, 60, 78, 105, 123 and 124 are believed to be full-length sequences.

BLASTN Polynucleotide Analysis

The isolated cDNA sequences were compared to sequences in the EMBL DNA database using the computer algorithm BLASTN. Comparisons of DNA sequences provided in SEQ ID NOS: 1-131, to sequences in the EMBL DNA database (using BLASTN) were made as of August, 2000, using Version 2.0.11 [Jan. 20, 2000], and the following Unix runing command: blastall -p blastn -d embldb -e 10 -G0 -E0 -r 1 -v 30 -b 30 -i queryseq -o.

The sequences of SEQ ID NOS: 11, 36, 51, 60, 61, 66, 70, 83, 90, 95, 98, 105, 117 and 126 were determined to have less than 50% identity, determined as described above, to sequences in the EMBL database using the computer algorithm BLASTN. The sequences of SEQ ID NOS: 5, 17, 27, 31, 56, 65, 79, 82, 84-87, 89, 91, 93, 94, 97, 109, 112, 115, 116, 118, 119, 123, 124 and 128 were determined to have less than 75% identity, determined as described above, to sequences in the EMBL database using the computer algorithm BLASTN. The sequences of SEQ ID NOS: 8, 12, 13, 21, 25, 29, 34, 35, 37, 41, 42, 44-46, 49, 52, 59, 62, 64, 67, 73, 74, 76-78, 80, 92, 100, 102, 106, 107, 108, 113, 114, 120, 121, 127, 130 and 131 were determined to have less than 96% identity, determined as described above, to sequences in the EMBL database using the computer algorithm BLASTN. Finally, the sequences of SEQ ID NOS: 2, 4, 16, 18, 22, 26, 28, 33, 38, 43, 47, 50, 53, 54, 57, 63, 71, 75, 81, 96 99, 101, 103, 104, 111, 122, 125 and 129 were determined to have less than 98% identity, determined as described above, to sequences in the EMBL database using the computer algorithm BLASTN.

The sequences of SEQ ID NOS: 182, 188, 191, 192, 198 and 210 were determined to have less than 50% identity, determined as described above, to sequences in the SwissProt database using the computer algorithm BLASTP. The sequences of SEQ ID NOS: 144, 148, 152, 162, 179, 180, 184, 185, 187, 190, 195, 201, 206, 213, 229, 236, 237, 239, 240, 249, 252 and 259 were determined to have less than 75% identity, determined as described above, to sequences in the SwissProt database using the computer algorithm BLASTP. The sequences of SEQ ID NOS: 136, 128, 142, 145, 153, 155, 160, 166, 167, 175, 177, 181, 183, 204, 208, 214, 216, 221, 223-226, 233, 241, 244, 245, 247, 248 and 250 were determined to have less than 90% identity, determine& as described above, to sequences in the SwissProt database using the computer algorithm BLASTP. Finally, the sequences of SEQ ID NOS: 133-135, 137, 140, 141, 150, 154, 157-159, 161, 163, 165, 168, 170, 172, 173, 176, 178, 194, 196, 197, 199, 202, 207, 211, 217, 220, 228, 234, 243, 253, 255, 257, 258, 260 and 262 were determined to have less than 98% identity, determined as described above, to sequences in the SwissProt database using the computer algorithm BLASTP.

The sequences of SEQ ID NOS: 5, 17, 27, 31, 51, 52-54, 56, 57, 60, 61, 67, 79, 82-86, 90, 98, 117, 124 and 126 were determined to have less than 50% identity, determined as described above, to sequences in the SwissProt database using the computer algorithm BLASTX. The sequences of SEQ ID NOS: 2, 8, 9, 11, 13, 14, 16, 18-22, 25, 26, 28, 36, 41-43, 48, 49, 59, 64, 68, 70, 72, 75, 78, 87, 93, 105, 106, 108-110, 112, 116, 118, 119, 121 and 123 were determined to have less than 75% identity, determined as described above, to sequences in the SwissProt database using the computer algorithm BLASTX. The sequences of SEQ ID NOS: 1, 3, 4, 7, 10, 12, 24, 29, 32-35, 44, 46, 47, 50, 63, 66, 69, 77, 91, 92, 94, 95, 97, 100-102, 113, 114, 120, 125, 128, 130 and 131 were determined to have less than 90% identity, determined as described above, to sequences in the SwissProt database using the computer algorithm BLASTX. Finally, the sequences of SEQ ID NOS: 6, 23, 30, 37-39, 45, 65, 71, 76, 80, 88, 89, 103, 107, 111, 122, 127 and 129 were determined to have less than 98% identity, determined as described above, to sequences in the SwissProt database using the computer algorithm BLASTX.

### EXAMPLE 2

### Expression of mRNA in Bovine Mammary Tissue

RNA was extracted from mammary gland tissue obtained from a non-pregnant heifer (Friesian Hereford cross, 2.5 years of age), a pregnant cow (Angus breed, 85 days pre-partum) and a lactating cow (Jersey breed, late lactating, non-pregnant and 2 hours post-milking), as well as from bovine liver, forebrain and kidney from an Angus Friesian cross heifer, using TRIzol (Gibco BRL, Gaithersburg, Md.) following the manufacturer's protocol. Sets of the various total RNA samples were run on 1.2% agarose/formaldehyde gels, 5 μg/lane. Following transfer to nitrocellulose membranes, RNA was cross-linked with ultraviolet light.

DNA probes were prepared from bacterial clones transformed with cDNA corresponding to SEQ ID NOS: 31, 32, 51, 90, 98, 105 and 124 by excision of the insert of the cDNA clone using EcoRI and XhoI restriction endonucleases, or by PCR amplification of the insert of the cDNA clone using T7 and T3 primers (Gibco BRL), or by using the entire cDNA clone. Probes were radiolabeled with α-P³²-dATP using Rediprime DNA labeling kits (Amersham Pharmacia Biotech, Uppsala, Sweden). Blots were hybridized overnight with rotation at 65° C. in a buffer containing 10-20 ml of 500 mM NaH₂PO₄, 1 mM EDTA, 7% SDS and then washed for 15 minutes at 65° C., first in 2×SSC/0.1% SDS and then in 1×SSC/0.1% SDS. The blots were exposed to Kodak XAR X-ray film for appropriate times.

The insert of the cDNA clone corresponding to SEQ ID NO: 14 hybridized strongly with transcripts of approximately 1.0 kb and 1.5 kb in the lactating mammary and liver samples. In the mammary sample the larger transcript predominated whereas in the liver the smaller transcript predominated. Only low levels of hybridization of the smaller transcript were, detected for the mammary samples from the non-pregnant, non-lactating and the pregnant cows.

The insert of the cDNA clone corresponding to SEQ ID NO: 29 hybridized with a transcript of approximately 1.8 kb in all the samples with the strongest levels being detected for the mammary samples from the non-pregnant, non-lactating and the pregnant cows. A second transcript of approximately 1.0 kb was detected in the lactating mammary gland sample only.

The insert of the cDNA clone corresponding to SEQ ID NO: 31 hybridized with transcripts of approximately 1.3 kb and 4.0 kb in the lactating mammary gland sample. No transcripts could be detected in the other tissue samples.

The insert of the cDNA clone corresponding to SEQ ID NO: 32 hybridized strongly with transcripts of approximately 1.0 kb and 1.5 kb in the lactating mammary and liver samples. In the mammary sample the larger transcript predominated whereas in the liver the smaller transcript predominated. Only low levels of hybridization of the smaller transcript were detected for the mammary samples from the non-pregnant, non-lactating and the pregnant cows.

The insert of the cDNA clone corresponding to SEQ ID NO 51 hybridized strongly to a transcript of approximately 1.0 kb in the lactating mammary sample. Much weaker hybridization was detected With transcripts of the same size in mammary samples from the non-pregnant, non-lactating and pregnant animal. No transcripts were detected in the liver, brain or kidney.

The insert of the cDNA clone corresponding to SEQ ID NO: 90 hybridized with a transcript of approximately 1.4 kb in the lactating mammary gland sample only.

The insert of the cDNA clone corresponding to SEQ ID NO: 98 hybridized strongly with a transcripts of approximately 0.8 kb and 1.4 kb in the lactating mammary gland sample only.

The insert of the cDNA clone corresponding to SEQ ID NO: 105 hybridized strongly with a transcript of approximately 1.0 kb and less strongly with a transcript of approximately 1.8 kb in the lactating mammary gland sample. Weaker hybridization was detected in the mammary samples from the non-pregnant, non lactating and the pregnant cows. No transcripts could be detected in the other tissue samples.

The insert of the cDNA clone corresponding to SEQ ID NO: 124 hybridized strongly with transcripts of approximately 1.0 kb and 1.4 kb in the lactating mammary gland sample. Lower levels of the larger transcript were detected in the brain and kidney samples.

In subsequent Northern blot experiments, the polypeptide of SEQ ID NO: 162 (encoded by SEQ ID NO: 31) was found to be expressed in mastitic, involuting and pregnant mammary gland tissue and in salivary gland.

SEQ ID NOS: 1-262 are set out in the attached Sequence Listing. The codes for nucleotide sequences used in the attached Sequence Listing, including the symbol “n,” conform to WIPO Standard ST.25 (1998), Appendix 2, Table 1.

All references cited herein, including patent references and non-patent publications, are hereby incorporated by reference in their entireties.

While in the foregoing specification this invention has been described in relation to certain preferred embodiments, and many details have been set forth for purposes of illustration, it will be apparent to those skilled in the art that the invention is susceptible to additional embodiments and that certain of the details described herein may be varied considerably without departing from the basic principles of the invention.

1. An isolated polynucleotide comprising a sequence selected from the group consisting of: SEQ ID NO: 1-131.

2. An isolated polynucleotide comprising a sequence selected from the group consisting of:
(a) complements of a sequence of SEQ ID NOS: 1-131;
(b) reverse complements Of a sequence of SEQ ID NOS: 1-131; and
(c) reverse sequences of the sequences recited in SEQ ID NOS: 1-131.

3. An isolated polynucleotide comprising a sequence selected from the group consisting of:
(a) sequences having at least 75% identity to a sequence of SEQ ID NO: 1-131;
(b) sequences having at least 90% identity to sequence of SEQ ID NO: 1-131;
(c) sequences having at least 95% identity to a sequence of SEQ ID NO: 1-131;
(d) nucleotide sequences that hybridize to a sequence of SEQ ID NO: 1-131 above under stringent hybridization conditions; and
(e) sequences that are degeneratively equivalent to a sequence of SEQ ID NO: 1-131, wherein the isolated polynucleotide encodes a polypeptide having substantially the same functional properties as a polypeptide of SEQ ID NO: 132-262.

4. An isolated polynucleotide comprising a sequence selected from the group consisting of:
(a) nucleotide sequences that are 200-mers of a sequence of SEQ ID NO: 1-131;
(b) nucleotide sequences that are 100-mers of a sequence of SEQ ID NO: 1-131;
(c) nucleotide sequences that are 40-mers of a sequence of SEQ ID NO: 1-131; and
(d) nucleotide sequences that are 20-mers of a sequence of SEQ ID NO: 1-131.

5. An oligonucleotide comprising at least 20 contiguous residues complementary to 20 contiguous residues of a nucleotide sequence of SEQ ID NO: 1-131.

6. A genetic construct comprising an isolated polynucleotide of any one of claims 1-4.

7. A host cell transformed with a genetic construct of claim 6.

8. An isolated polypeptide encoded by a polynucleotide of claim 1.

9. An isolated polypeptide comprising an amino acid sequence selected from the group consisting of: SEQ ID NO: 132-262.

10. An isolated polypeptide comprising an amino acid sequence selected from the group consisting of:
(a) sequences having at least 75% identity to a sequence of SEQ ID NO: 132-262;
(b) sequences having at least 90% identity to a sequence of SEQ ID NO: 132-262; and
(c) sequences having at least 95% identity to a sequence of SEQ ID NO: 132-262,
wherein the isolated polypeptide has substantially the same functional properties as a polypeptide of SEQ ID NO: 132-262.

11. An isolated polynucleotide encoding a polypeptide of claim 10.

12. An isolated polypeptide comprising at least a functional portion of an amino acid sequence of SEQ ID NO: 132-262.

13. A composition comprising a polypeptide according to any one of claims 9 and 10 and at least one component selected from the group consisting of: physiologically acceptable carriers and immunostimulants.

14. A composition comprising a polynucleotide according to claim 1 and at least one component selected from the group consisting of pharmaceutically acceptable carriers and immunostimulants.

15. A method for treating a disorder in a mammal comprising administering a composition according to claim 13.

16. A method for treating a disorder in a mammal comprising administering a composition according to claim 14.

17. A method for modifying mammary gland function, structure or composition in an organism, comprising transforming the organism with a genetic construct according to claim.

18. A method for modifying mammary gland function, or milk composition in an organism, comprising administering a composition according to claim 13.

19. A method for detecting the presence of mammary gland tissue in a biological sample, comprising:
(a) contacting the biological sample with an oligonucleotide according to claim 5;
(b) detecting in the sample the presence of a polynucleotide that hybridizes to the oligonucleotide.

20. A diagnostic kit comprising at least one oligonucleotide according to claim 5.

21. A transgenic organism comprising a host cell according to claim 7.
